(19) United States
(12) Patent Application Publication
(10) Pub. No.: US 2023/0036867 A1
(43) Pub. Date: Feb. 2, 2023

(54) N-(3-(5-(PYRIMIDIN-4-YL)THIAZOL-4-YL)PHENYL)SULFONAMIDE COMPOUNDS AND THEIR USES AS BRAF INHIBITORS

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE - CNRS, Paris (FR); UNIVERSITE DE MONTPELLIER, Montpellier (FR); INSTITUT REGIONAL DU CANCER DE MONTPELLIER - VAL D'AURELLE, Montpellier (FR)

(72) Inventors: Gilles LABESSE, Montpellier (FR); Melanie SCHNEIDER, Hinxton, Cambridgeshire (GB); Muriel GELIN, Montpellier (FR); Jean-François Alexandre GUICHOU, Montpellier (FR); Martin COHEN-GONSAUD, Montpellier (FR); William BOURGUET, Montpellier (FR); Patrick BALAGUER, Montpellier (FR)

(21) Appl. No.: 17/782,485

(22) PCT Filed: Dec. 5, 2020
(86) PCT No.: PCT/EP2020/084778

§ 371 Date: Jun. 3, 2022

(30) Foreign Application Priority Data
Dec. 5, 2019 (EP) 19306579.4

(51) Int. Cl. C07D 417/04; C07D 417/14

(52) U.S. Cl. CPC C07D 417/04; C07D 417/14

(57) ABSTRACT

The present invention relates to N-(3-(5-(pyrimidin-4-yl)thiazol-4-yl)phenylsulfonamide compounds which are useful as inhibitors of protein kinases, more specifically BRAF or mutant forms thereof, to pharmaceutical composition comprising such compounds, and to uses of such compounds in the treatment or prevention of diseases associated with deregulated protein kinase activity, such as cancer.

The present invention relates to N-(3-(5-(pyrimidin-4-yl)thiazol-4-yl)phenylsulfonamide compounds which are useful as inhibitors of protein kinases, more specifically BRAF or mutant forms thereof, to pharmaceutical composition comprising such compounds, and to uses of such compounds in the treatment or prevention of diseases associated with deregulated protein kinase activity, such as cancer.

### BACKGROUND OF THE INVENTION

The protein kinases represent a large family of proteins, which play a central role in the regulation of a wide variety of cellular processes and maintaining control over cellular function. Protein kinases include tyrosine kinases and serine/threonine kinases. Deregulated protein kinase activity has been observed in many diseases including benign and malignant proliferative disorders as well as diseases resulting from inappropriate activation of the immune and nervous systems.

BRAF is one of the three isoforms (with CRAF and ARAF) of the Rapidly Accelerated Fibrosarcoma (RAF) family of catalytically competent serine/threonine protein kinases (two pseudokinases, KSR1 and KSR2, are also included in the RAF family). BRAF plays a vital role in the RAS/RAF/MEK/ERK signalling cascade, which is also known as mitogen-activated protein kinase (MAPK) pathway, and participates in cell proliferation and survival (M. J. Robinson et al., Curr. Opin. Cell Biol., 1997, 9, 180-186). Upon induction of conformational changes by RAS binding, stimulating the formation of active RAF homodimers or heterodimers, RAF changes its phosphorylation status, which triggers its kinase activity that activates MEK (MEK1 and MEK2), which in turn phosphorylates downstream ERK (ERK1 and ERK2). In contrast to the RAF and MEK kinases, ERK has a broad substrate specificity and is able to phosphorylate hundreds of different proteins (R. Roskoski, Pharmacol. Res., 2015, 100, 1-23). As RAS is mutated in approximately 30% of human cancers, the development of inhibitors has been under investigation for a long while, but without significant success (R. Roskoski, Pharmacol. Res., 2018, 135, 239-258). In addition, the oncogenic activation of BRAF induces constitutively and RAS-independently the MAPK pathway leading to the uncontrolled amplification of downstream signalling, which involves an increase of proliferation and finally tumorigenesis (H. Davies et al., Nature, 2002, 417, 949-954). Many mutations (>30) of the BRAF gene associated with human cancers have been identified (P. T. C. Wan et al., Cell, 2004, 116, 855-867). These are involved in approximately 100% of hairy cell leukaemia (B. Falini et al., Blood, 2016, 128, 1918-1927), 50% of melanomas, 45% of thyroid, 10% of colon, and 8% of ovarian carcinomas (M. Pulici, ChemMedChem, 2015, 10, 276-295). The most common mutation, accounting for approximately 90% of the detected BRAF mutated cases, is the replacement of valine with glutamic acid at position 600 (shortly V600E), which is located within the activation segment of the kinase domain and destabilizes the inactive conformation. This mutation leads to a constitutive kinase activity that is about 500-fold increased compared to wild type (WT) BRAF. Moreover, in contrast to the WT, BRAF-V600E is signalling as a monomer and insensitive to ERK negative feedback mechanisms (C. A. Pratilas, Proc. Natl. Acad. Sci. USA, 2009, 106, 4519-4524). Therefore, inhibiting mutant forms of BRAF, such as BRAF-V600E for example, is a promising strategy for cancer treatment.

BRAF inhibitors, such as vermurafenib (P. B. Chapman et al., New Engl. J. Med., 2011, 364, 2507-2516), sorafenib (P. T. C. Wan et al., Cell, 2004, 116, 855-867), and dabrafenib (G. T. Gibney et al., Expert. Opin. Drug. Metab. Toxicol., 2013, 9, 893-899) have been developed in order to block the MAPK signalling pathway and decrease tumor cell growth in cells expressing the BRAF mutant V600E. Selective targeting of BRAF-V600E is a proven therapeutic strategy for the treatment of metastatic melanoma and the drugs vemurafenib and dabrafenib have been approved by the U.S. Food and Drug Administration (FDA) for treatment of late-stage melanoma in 2011 and 2013, respectively (G. Kim et al.; Clin. Cancer Res., 2014, 20, 4994-5000; A. D. Ballantyne et al., Drugs, 2013, 76, 1367-1376; A. M. Menzies et al., Clin. Cancer Res., 2014, 20, 2035-2043). Both drugs show improved response rates and overall survival of BRAF-V600E mutant melanoma patients, but unfortunately, due to rapidly acquired resistance most patients relapse within a year (W. Zhang, Curr. Opin. Pharmacol, 2015, 23, 68-73).

Dabrafenib is a potent and selective inhibitor for BRAF-V600E, but it has been found that its bioavailability decreases rather rapidly (with a half-life of 5 hours), which is likely due to induction of its own metabolism through cytochrome P450s (CYPs) Dabrafenib metabolism is mediated by CYP3A4 and CYP2C8. Thus, Dabrafenib is supposed to be subject of drug-drug interactions with strong inhibitors of CYP2C8 and/or CYP3A4. CYP3A4 and CYP2B6 mRNA induction is indicating interactions of Dabrafenib with the nuclear receptors Pregnane X Receptor (PXR) and/or Constitutive Androstane Receptor (CAR) (C. L. Denton et al., J. Clin. Pharmacol, 2013, 53, 955-961; D. A. Bershas et al., Drug Metab. Dispos, 2013, 41, 2215-2224; S. K. Lawrence et al., Drug Metab. Dispos, 2014, 42, 1180-1190; D. Ouellet, J. Clin. Pharmacol., 2014, 54, 696-706; J. Gil, et al., Cell Biol. Toxicol, 2019; A. Puszkiel et al. Clin. Pharmacokinetics, 2019, 58, 451-467).

The Pregnane X Receptor (PXR) belonging to NR subfamily I, plays an unusual and outstanding role as master regulator for xenobiotic metabolism. It is responsible for the organism's defense against foreign substances and therefore a main regulator for detoxification, acting as sensor to a broad spectrum of ligands (endogenous metabolites, drugs and xenobiotics) with very diverse characteristics (concerning composition, shape and size). Unfortunately, undesired drug binding to PXR is causing many adverse effects. PXR forms heterodimers with the Retinoid X Receptor α (RXRα) and subsequently binds to PXR responsive elements. As main transcriptional inducer of cytochrome P450 enzyme CYP3A4, one of the main metabolizing enzymes for many drugs in clinical use, it acts as a key player for inducing drug degradation and can potentially cause undesirable drug-drug interactions (T. M. Willson et al., Nature Rev. Drug Discov., 2002, 1, 259-266). Rapid metabolism decreases efficacy for many drugs, but drugs with active metabolites can display increased drug effect and/or toxicity upon metabolism. Undesirable drug-drug interactions are also a metabolic issue. When two drugs sharing a metabolism pathway via the same enzyme compete for the same binding site, the one with higher potency predominates and the metabolism of the competing drug is decreased. This, in turn, can lead to increased risks for toxic effects of the unmetabolized compound, as serum levels may be elevated. PXR is also widely expressed in many different tumors (breast, colon, prostate and ovary) where it has been shown to be involved in both the development of multi-drug resistance and enhanced cancer cells aggressiveness (A. Geick et al., J. Biol. Chem., 2001, 276, 14581-14587). An increasing number of drugs are clinically tested in cancers with sometimes rather limited success and it was also shown recently that some of them could be direct ligands of PXR, thereby inducing their own metabolism or the metabolism of co-administered drugs. PXR is classified as unwanted and harmful secondary target whose activation needs to be avoided in order to simultaneously avoid the activation of the degradation pathway via CYP450 enzymes. Accordingly, a limited interaction with PXR is required additionally to a drug's efficient binding to its primary target. Therefore, an improvement of drugs includes a fine tuning with chemical changes that do not perturb other important characteristics, such as stability, bioavailability, etc., but prevent PXR binding.

Examples of BRAF inhibitors are disclosed in patent applications US 2009/0298815 A1, US 2011/0306625 A1, WO 2011/161216 A1, WO 2012/113774 A1 and WO 2012/125981 A2, but the absence of binding to PXR has not been demonstrated.

Thus, there is still a need for compounds active as protein kinase inhibitors, but without activating PXR.

### SUMMARY OF THE INVENTION

The inventors have now succeeded in developing compounds of formula I, described below, that are useful in therapy as anticancer agents.

These compounds have the advantage of inhibiting protein kinases, particularly serine/threonine kinases, more particularly BRAF or mutants thereof, without activating PXR.

The invention therefore relates to compounds of general Formula I, their pharmaceutically acceptable salts or solvates thereof, as well as methods of use of such compounds or compositions comprising such compounds as inhibitors of protein kinases, particularly serine/threonine kinases, more particularly BRAF or mutants thereof.

In a general aspect, the invention provides compounds of general Formula I:

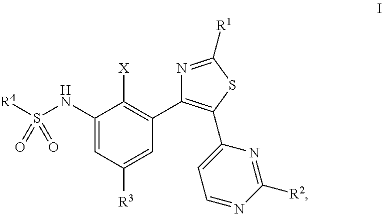

a pharmaceutically acceptable salt or a solvate thereof,

wherein

X is halogen;

R¹ is selected from the group consisting of C1-C6-alkyl, amino-C1-C6-alkyl, piperidinyl, morpholinyl, piperazinyl, pyrrolidinyl and azetidinyl, said amino-C1-C6-alkyl, piperidinyl, morpholinyl, piperazinyl, pyrrolidinyl and azetidinyl being linked to the thiazol ring through a carbon atom and optionally substituted by C1-C6-alkyl, C3-C6-cycloalkyl or C1-C4-alkyloxycarbonyl;

R² is selected from the group consisting of C1-C6-alkyl, halogen and NHR⁵, wherein R⁵ is selected from the group consisting of H, —C(O)—C1-C6-alkyl, —C(O)—C1-C6-alkenyl and —C(O)—C1-C6-alkynyl;

R³ is selected from the group consisting of H, C1-C6-alkyl and halogen; and

R⁴ is selected from the group consisting of C1-C6-alkyl and dihalogenoaryl;

with the proviso that R¹ is not C1-C6-alkyl when one of R², R³ and R⁴ is C1-C6-alkyl or when R³ is H.

In another aspect, the present invention provides a pharmaceutical composition comprising at least one compound according to the invention or a pharmaceutically acceptable salt or solvate thereof and at least one pharmaceutically acceptable carrier, diluent, excipient and/or adjuvant.

The invention further relates to compounds of Formula I or their pharmaceutically acceptable salts or solvates thereof for use in treating or preventing a disease associated with deregulated protein kinase activity.

### DETAILED DESCRIPTION OF THE INVENTION

As detailed above, the invention relates to compounds of Formula I, as well as their pharmaceutically acceptable salts or solvates.

Preferred compounds of Formula I or pharmaceutically acceptable salts or solvates thereof are those wherein one or more of X, R¹, R², R³ and R⁴ are defined as follows:

X is halogen; particularly X is chloro or fluoro; more particularly X is fluoro;

R¹ is selected from the group consisting of C1-C6-alkyl, amino-C1-C6-alkyl, piperidinyl, morpholinyl, piperazinyl, pyrrolidinyl and azetidinyl, said amino-C1-C6-alkyl, piperidinyl, morpholinyl, piperazinyl, pyrrolidinyl and azetidinyl being linked to the thiazol ring through a carbon atom and optionally substituted by C1-C6-alkyl, C3-C6-cycloalkyl or C1-C4-alkyloxycarbonyl; particularly R¹ is selected from the group consisting of C1-C4-alkyl, amino-C1-C4-alkyl, piperidinyl, morpholinyl, piperazinyl, pyrrolidinyl and azetidinyl, said amino-C1-C4-alkyl, piperidinyl, morpholinyl, piperazinyl, pyrrolidinyl and azetidinyl being linked to the thiazol ring through a carbon atom and optionally substituted by C1-C4-alkyl, C3-C6-cycloalkyl or C1-C4-alkyloxycarbonyl; more particularly R¹ is selected from the group consisting of C2-C4-alkyl, amino-C1-C3-alkyl, piperidin-4-yl, piperidin-3-yl, morpholin-3-yl, morpholin-2-yl, piperazin-2-yl, pyrrolidin-3-yl, pyrrolidin-2-yl, azetidin-3-yl and azetidin-2-yl, said amino-C1-C3-alkyl, piperidin-4-yl, piperidin-3-yl, morpholin-3-yl, morpholin-2-yl, piperazin-2-yl, pyrrolidin-3-yl, pyrrolidin-2-yl, azetidin-3-yl and azetidin-2-yl being optionally substituted by C1-C4-alkyl, C3-C6-cycloalkyl or C1-C4-alkyloxycarbonyl; even more particularly R¹ is selected from the group consisting of tert-butyl, 3-aminopropyl, piperidin-4-yl, piperidin-3-yl, morpholin-3-yl, piperazin-2-yl, pyrrolidin-2-yl and azetidin-2-yl, said 3-aminopropyl, piperidin-4-yl, piperidin-3-yl, morpholin-3-yl, piperazin-2-yl, pyrrolidin-2-yl and azetidin-2-yl being optionally N-substituted by C3-C6-cycloalkyl or C1-C4-alkyloxycarbonyl; still more particularly R¹ is selected from the group consisting of tert-butyl, 3-aminopropyl, piperidin-4-yl, piperidin-3-yl, morpholin-3-yl, piperazin-2-yl, pyrrolidin-2-yl and azetidin-2-yl, said piperidin-4-yl and piperidin-3-yl being optionally N-substituted by cyclopropyl or tert-butyloxycarbonyl; for example, R¹ is selected from the group consisting of tert-butyl, 3-aminopropyl, piperidin-4-yl, piperidin-3-yl, morpholin-3-yl, piperazin-2-yl, pyrrolidin-2-yl and azetidin-2-yl, said 3-aminopropyl, piperidin-4-yl, piperidin-3-yl, morpholin-3-yl, piperazin-2-yl, pyrrolidin-2-yl and azetidin-2-yl being optionally N-substituted by C3-C6-cycloalkyl or C1-C4-alkyloxycarbonyl or C-substituted by C1-C4-alkyl; in another example, R¹ is selected from the group consisting of tert-butyl, 3-aminopropyl, piperidin-4-yl, piperidin-3-yl, morpholin-3-yl, piperazin-2-yl, pyrrolidin-2-yl and azetidin-2-yl, said piperidin-4-yl, piperidin-3-yl and piperazin-2-yl being optionally N-substituted by cyclopropyl or tert-butyloxycarbonyl and said morpholin-3-yl being C-disubstituted by methyl;

R² is selected from the group consisting of C1-C6-alkyl, halogen and NHR⁵, wherein R⁵ is selected from the group consisting of H, —C(O)—C1-C6-alkyl, —C(O)—C1-C6-alkenyl and —C(O)—C1-C6-alkynyl; particularly R² is selected from the group consisting of C1-C4-alkyl, fluoro, chloro and NHR⁵, wherein R⁵ is selected from the group consisting of H, —C(O)—C1-C6-alkyl, —C(O)—C1-C6-alkenyl and —C(O)—C1-C6-alkynyl; more particularly R² is NHR⁵, wherein R⁵ is selected from the group consisting of H, —C(O)—C1-C6-alkyl, —C(O)—C1-C6-alkenyl and —C(O)—C1-C6-alkynyl; even more particularly R² is NHR⁵, wherein R⁵ is selected from the group consisting of H, —C(O)—C1-C4-alkyl, —C(O)—C1-C4-alkenyl and —C(O)—C1-C4-alkynyl; still more particularly R² is NHR⁵, wherein R⁵ is selected from the group consisting of H, —C(O)—C1-C2-alkyl, —C(O)—CH═CH₂ and —C(O)—C≡CH; still more particularly R² is NHR⁵, wherein R⁵ is selected from the group consisting of H, —C(O)Me and —C(O)—CH═CH₂;

R³ is selected from the group consisting of H, C1-C6-alkyl and halogen; particularly R³ is selected from the group consisting of H, C1-C4-alkyl and halogen; more particularly R³ is selected from the group consisting of H, C1-C2-alkyl, fluoro and chloro; even more particularly R³ is H or chloro;

R⁴ is selected from the group consisting of C1-C6-alkyl and dihalogenoaryl; particularly,

R⁴ is selected from the group consisting of C1-C6-alkyl and dihalogenophenyl; more particularly, R⁴ is selected from the group consisting of C1-C6-alkyl and 2,5-dihalogenophenyl; even more particularly, R⁴ is selected from the group consisting of C2-C6-alkyl, 2,5-difluorophenyl and 2,5-dichlorophenyl; still more particularly, R⁴ is C2-C4-alkyl or 2,5-difluorophenyl; for example, R⁴ is selected from the group consisting of C4-C6-alkyl and 2,5-dihalogenophenyl; in another example, R⁴ is selected from the group consisting of C4-alkyl and 2,5-dihalogenophenyl; in another example, R⁴ is selected from the group consisting of sec-butyl and 2,5-difluorophenyl.

In one embodiment, the compounds of Formula I are those wherein X is fluoro.

In one embodiment, the compounds of Formula I are those wherein R² is NHR⁵, wherein R⁵ is as defined above.

In one embodiment, the compounds of Formula I are those wherein R² is NHR⁵, wherein R⁵ is H.

In one embodiment, the compounds of Formula I are those wherein R² is NHR⁵, wherein R⁵ is selected from the group consisting of H, —C(O)Me, —C(O)—CH═CH₂ and —C(O)—C≡CH, particularly R⁵ is selected from the group consisting of H, —C(O)Me and —C(O)—CH═CH₂, more particularly R⁵ is H or —C(O)Me.

In one embodiment, the compounds of Formula I are those wherein R³ is H or chloro.

In one embodiment, the compounds of Formula I are those wherein R³ is H.

In one embodiment, the compounds of Formula I are those wherein R³ is chloro.

In one embodiment, the compounds of Formula I are those wherein R⁴ is selected from the group consisting of C1-C6-alkyl and 2,5-dihalogenophenyl, particularly R⁴ is selected from the group consisting of C2-C6-alkyl and 2,5-difluorophenyl, more particularly R⁴ is selected from the group consisting of C2-C4-alkyl and 2,5-difluorophenyl; still more particularly, R⁴ is C4-alkyl or 2,5-difluorophenyl.

In one embodiment, the compounds of Formula I are those wherein R⁴ is selected from the group consisting of C1-C2-alkyl, C4-C6-alkyl and 2,5-dihalogenophenyl, particularly R⁴ is selected from the group consisting of C4-C5-alkyl and 2,5-difluorophenyl, more particularly R⁴ is selected from the group consisting of C4-alkyl and 2,5-difluorophenyl; still more particularly, R⁴ is sec-butyl or 2,5-difluorophenyl.

In one embodiment, the compounds of Formula I are those wherein R⁴ is C1-C6-alkyl, particularly R⁴ is C2-C6-alkyl, more particularly R⁴ is C2-C4-alkyl, still more particularly R⁴ is C4-alkyl.

In one embodiment, the compounds of Formula I are those wherein R⁴ selected from the group consisting of C1-C2-alkyl and C4-C6-alkyl, particularly R⁴ is C4-C6-alkyl, more particularly R⁴ is C4-C5-alkyl, still more particularly R⁴ is C4-alkyl, even more particularly, R⁴ is sec-butyl.

In one embodiment, the compounds of Formula I are those wherein R⁴ is 2,5-dihalogenophenyl, particularly R⁴ is 2,5-difluorophenyl.

In one embodiment, the compounds of Formula I are those of Formula II:

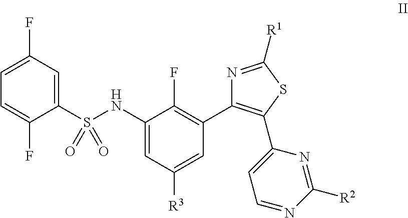

or pharmaceutically acceptable salts or solvates thereof,

wherein

R¹, R² and R³ are as defined above with respect to Formula I and any of its embodiments.

Preferred compounds of Formula II or pharmaceutically acceptable salts or solvates thereof are those wherein one or more of R¹, R² and R³ are defined as follows:

R¹ is selected from the group consisting of C1-C6-alkyl, amino-C1-C6-alkyl, piperidinyl, morpholinyl, piperazinyl, pyrrolidinyl and azetidinyl, said amino-C1-C6-alkyl, piperidinyl, morpholinyl, piperazinyl, pyrrolidinyl and azetidinyl being linked to the thiazol ring through a carbon atom and optionally substituted by C1-C6-alkyl, C3-C6-cycloalkyl or C1-C4-alkyloxycarbonyl; particularly R¹ is selected from the group consisting of C1-C4-alkyl, amino-C1-C4-alkyl, piperidinyl, morpholinyl, piperazinyl, pyrrolidinyl and azetidinyl, said amino-C1-C4-alkyl, piperidinyl, morpholinyl, piperazinyl, pyrrolidinyl and azetidinyl being linked to the thiazol ring through a carbon atom and optionally substituted by C1-C4-alkyl, C3-C6-cycloalkyl or C1-C4-alkyloxycarbonyl; more particularly R¹ is selected from the group consisting of C2-C4-alkyl, amino-C1-C3-alkyl, piperidin-4-yl, piperidin-3-yl, morpholin-3-yl, morpholin-2-yl, piperazin-2-yl, pyrrolidin-3-yl, pyrrolidin-2-yl, azetidin-3-yl and azetidin-2-yl, said amino-C1-C3-alkyl, piperidin-4-yl, piperidin-3-yl, morpholin-3-yl, morpholin-2-yl, piperazin-2-yl, pyrrolidin-3-yl, pyrrolidin-2-yl, azetidin-3-yl and azetidin-2-yl being optionally substituted by C1-C4-alkyl, C3-C6-cycloalkyl or C1-C4-alkyloxycarbonyl; even more particularly R¹ is selected from the group consisting of tert-butyl, 3-aminopropyl, piperidin-4-yl, piperidin-3-yl, morpholin-3-yl, piperazin-2-yl, pyrrolidin-2-yl and azetidin-2-yl, said 3-aminopropyl, piperidin-4-yl, piperidin-3-yl, morpholin-3-yl, piperazin-2-yl, pyrrolidin-2-yl and azetidin-2-yl being optionally N-substituted by C3-C6-cycloalkyl or C1-C4-alkyloxycarbonyl; still more particularly R¹ is selected from the group consisting of tert-butyl, 3-aminopropyl, piperidin-4-yl, piperidin-3-yl, morpholin-3-yl, piperazin-2-yl, pyrrolidin-2-yl and azetidin-2-yl, said piperidin-4-yl and piperidin-3-yl being optionally N-substituted by cyclopropyl or tert-butyloxycarbonyl; for example, R¹ is selected from the group consisting of tert-butyl, 3-aminopropyl, piperidin-4-yl, piperidin-3-yl, morpholin-3-yl, piperazin-2-yl, pyrrolidin-2-yl and azetidin-2-yl, said 3-aminopropyl, piperidin-4-yl, piperidin-3-yl, morpholin-3-yl, piperazin-2-yl, pyrrolidin-2-yl and azetidin-2-yl being optionally N-substituted by C3-C6-cycloalkyl or C1-C4-alkyloxycarbonyl or C-substituted by C1-C4-alkyl; in another example, R¹ is selected from the group consisting of tert-butyl, 3-aminopropyl, piperidin-4-yl, piperidin-3-yl, morpholin-3-yl, piperazin-2-yl, pyrrolidin-2-yl and azetidin-2-yl, said piperidin-4-yl, piperidin-3-yl and piperazin-2-yl being optionally N-substituted by cyclopropyl or tert-butyloxycarbonyl and said morpholin-3-yl being C-disubstituted by methyl;

R² is C1-C6-alkyl or NHR⁵, wherein R⁵ is selected from the group consisting of H, —C(O)—C1-C6-alkyl, —C(O)—C1-C6-alkenyl and —C(O)—C1-C6-alkynyl; particularly R² is C1-C4-alkyl or NHR⁵, wherein R⁵ is selected from the group consisting of H, —C(O)—C1-C6-alkyl, —C(O)—C1-C6-alkenyl and —C(O)—C1-C6-alkynyl; more particularly R² is NHR⁵, wherein R⁵ is selected from the group consisting of H, —C(O)—C1-C6-alkyl, —C(O)—C1-C6-alkenyl and —C(O)—C1-C6-alkynyl; even more particularly R² is NHR⁵, wherein R⁵ is selected from the group consisting of H, —C(O)—C1-C4-alkyl, —C(O)—C1-C4-alkenyl and —C(O)—C1-C4-alkynyl; still more particularly R² is NHR⁵, wherein R⁵ is selected from the group consisting of H, —C(O)—C1-C2-alkyl, —C(O)—CH═CH₂ and —C(O)—C≡CH; still more particularly R² is NHR⁵, wherein R⁵ is selected from the group consisting of H, —C(O)Me and —C(O)—CH═CH₂;

R³ is H or halogen; preferably R³ is H, fluoro or chloro; more preferably R³ is H or chloro.

In one embodiment, the compounds of Formula I are those of Formula III:

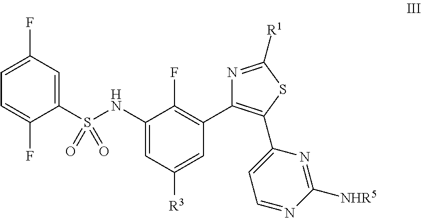

or pharmaceutically acceptable salts or solvates thereof,

wherein

R¹, R³ and R⁵ are as defined above with respect to Formula I and any of its embodiments.

Preferred compounds of Formula III or pharmaceutically acceptable salts or solvates thereof are those wherein one or more of R¹, R³ and R⁵ are defined as follows:

R¹ is selected from the group consisting of C1-C6-alkyl, amino-C1-C6-alkyl, piperidinyl, morpholinyl, piperazinyl, pyrrolidinyl and azetidinyl, said amino-C1-C6-alkyl, piperidinyl, morpholinyl, piperazinyl, pyrrolidinyl and azetidinyl being linked to the thiazol ring through a carbon atom and optionally substituted by C1-C6-alkyl, C3-C6-cycloalkyl or C1-C4-alkyloxycarbonyl; particularly R¹ is selected from the group consisting of C1-C4-alkyl, amino-C1-C4-alkyl, piperidinyl, morpholinyl, piperazinyl, pyrrolidinyl and azetidinyl, said amino-C1-C4-alkyl, piperidinyl, morpholinyl, piperazinyl, pyrrolidinyl and azetidinyl being linked to the thiazol ring through a carbon atom and optionally substituted by C1-C4-alkyl, C3-C6-cycloalkyl or C1-C4-alkyloxycarbonyl; more particularly R¹ is selected from the group consisting of C2-C4-alkyl, amino-C1-C3-alkyl, piperidin-4-yl, piperidin-3-yl, morpholin-3-yl, morpholin-2-yl, piperazin-2-yl, pyrrolidin-3-yl, pyrrolidin-2-yl, azetidin-3-yl and azetidin-2-yl, said amino-C1-C3-alkyl, piperidin-4-yl, piperidin-3-yl, morpholin-3-yl, morpholin-2-yl, piperazin-2-yl, pyrrolidin-3-yl, pyrrolidin-2-yl, azetidin-3-yl and azetidin-2-yl being optionally substituted by C1-C4-alkyl, C3-C6-cycloalkyl or C1-C4-alkyloxycarbonyl; even more particularly R¹ is selected from the group consisting of tert-butyl, 3-aminopropyl, piperidin-4-yl, piperidin-3-yl, morpholin-3-yl, piperazin-2-yl, pyrrolidin-2-yl and azetidin-2-yl, said 3-aminopropyl, piperidin-4-yl, piperidin-3-yl, morpholin-3-yl, piperazin-2-yl, pyrrolidin-2-yl and azetidin-2-yl being optionally N-substituted by C3-C6-cycloalkyl or C1-C4-alkyloxycarbonyl; still more particularly R¹ is selected from the group consisting of tert-butyl, 3-aminopropyl, piperidin-4-yl, piperidin-3-yl, morpholin-3-yl, piperazin-2-yl, pyrrolidin-2-yl and azetidin-2-yl, said piperidin-4-yl and piperidin-3-yl being optionally N-substituted by cyclopropyl or tert-butyloxycarbonyl; for example, R¹ is selected from the group consisting of tert-butyl, 3-aminopropyl, piperidin-4-yl, piperidin-3-yl, morpholin-3-yl, piperazin-2-yl, pyrrolidin-2-yl and azetidin-2-yl, said 3-aminopropyl, piperidin-4-yl, piperidin-3-yl, morpholin-3-yl, piperazin-2-yl, pyrrolidin-2-yl and azetidin-2-yl being optionally N-substituted by C3-C6-cycloalkyl or C1-C4-alkyloxycarbonyl or C-substituted by C1-C4-alkyl; in another example, R¹ is selected from the group consisting of tert-butyl, 3-aminopropyl, piperidin-4-yl, piperidin-3-yl, morpholin-3-yl, piperazin-2-yl, pyrrolidin-2-yl and azetidin-2-yl, said piperidin-4-yl, piperidin-3-yl and piperazin-2-yl being optionally N-substituted by cyclopropyl or tert-butyloxycarbonyl and said morpholin-3-yl being C-disubstituted by methyl;

R⁵ is selected from the group consisting of H, —C(O)—C1-C6-alkyl and —C(O)—C1-C6-alkenyl; particularly R⁵ is selected from the group consisting of H, —C(O)—C1-C4-alkyl and —C(O)—C1-C4-alkenyl; more particularly R⁵ is selected from the group consisting of H, —C(O)—C1-C2-alkyl and —C(O)—CH═CH₂; still more particularly R⁵ is selected from the group consisting of H, —C(O)Me and —C(O)—CH═CH₂;

R³ is H or halogen; particularly R³ is H, fluoro or chloro; more particularly R³ is H or chloro.

In one embodiment, the compounds of Formula I are those of Formula IV:

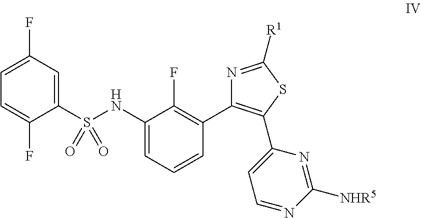

or pharmaceutically acceptable salts or solvates thereof,

wherein

R¹ and R⁵ are as defined above with respect to Formula I and any of its embodiments.

Preferred compounds of Formula IV or pharmaceutically acceptable salts or solvates thereof are those wherein one or more of R¹ and R⁵ are defined as follows:

R¹ is selected from the group consisting of amino-C1-C6-alkyl, piperidinyl, morpholinyl, piperazinyl, pyrrolidinyl and azetidinyl, said amino-C1-C6-alkyl, piperidinyl, morpholinyl, piperazinyl, pyrrolidinyl and azetidinyl being linked to the thiazol ring through a carbon atom and optionally substituted by C1-C6-alkyl, C3-C6-cycloalkyl or C1-C4-alkyloxycarbonyl; particularly R¹ is selected from the group consisting of amino-C1-C4-alkyl, piperidinyl, morpholinyl, piperazinyl, pyrrolidinyl and azetidinyl, said amino-C1-C4-alkyl, piperidinyl, morpholinyl, piperazinyl, pyrrolidinyl and azetidinyl being linked to the thiazol ring through a carbon atom and optionally substituted by C1-C4-alkyl, C3-C6-cycloalkyl or C1-C4-alkyloxycarbonyl; more particularly R¹ is selected from the group consisting of amino-C1-C3-alkyl, piperidin-4-yl, piperidin-3-yl, morpholin-3-yl, morpholin-2-yl, piperazin-2-yl, pyrrolidin-3-yl, pyrrolidin-2-yl, azetidin-3-yl and azetidin-2-yl, said amino-C1-C3-alkyl, piperidin-4-yl, piperidin-3-yl, morpholin-3-yl, morpholin-2-yl, piperazin-2-yl, pyrrolidin-3-yl, pyrrolidin-2-yl, azetidin-3-yl and azetidin-2-yl being optionally substituted by C1-C4-alkyl, C3-C6-cycloalkyl or C1-C4-alkyloxycarbonyl; even more particularly R¹ is selected from the group consisting of 3-aminopropyl, piperidin-4-yl, piperidin-3-yl, morpholin-3-yl, piperazin-2-yl, pyrrolidin-2-yl and azetidin-2-yl, said 3-aminopropyl, piperidin-4-yl, piperidin-3-yl, morpholin-3-yl, piperazin-2-yl, pyrrolidin-2-yl and azetidin-2-yl being optionally N-substituted by C3-C6-cycloalkyl or C1-C4-alkyloxycarbonyl; still more particularly R¹ is selected from the group consisting of 3-aminopropyl, piperidin-4-yl, piperidin-3-yl, morpholin-3-yl, piperazin-2-yl, pyrrolidin-2-yl and azetidin-2-yl, said piperidin-4-yl and piperidin-3-yl being optionally N-substituted by cyclopropyl or tert-butyloxycarbonyl; for example, R¹ is selected from the group consisting of 3-aminopropyl, piperidin-4-yl, piperidin-3-yl, morpholin-3-yl, piperazin-2-yl, pyrrolidin-2-yl and azetidin-2-yl, said 3-aminopropyl, piperidin-4-yl, piperidin-3-yl, morpholin-3-yl, piperazin-2-yl, pyrrolidin-2-yl and azetidin-2-yl being optionally N-substituted by C3-C6-cycloalkyl or C1-C4-alkyloxycarbonyl or C-substituted by C1-C4-alkyl; in another example, R¹ is selected from the group consisting of 3-aminopropyl, piperidin-4-yl, piperidin-3-yl, morpholin-3-yl, piperazin-2-yl, pyrrolidin-2-yl and azetidin-2-yl, said piperidin-4-yl, piperidin-3-yl and piperazin-2-yl being optionally N-substituted by cyclopropyl or tert-butyloxycarbonyl and said morpholin-3-yl being C-disubstituted by methyl;

R⁵ is selected from the group consisting of H, —C(O)—C1-C6-alkyl and —C(O)—C1-C6-alkenyl; particularly R⁵ is selected from the group consisting of H, —C(O)—C1-C4-alkyl, —C(O)—C1-C4-alkenyl; more particularly R⁵ is selected from the group consisting of H, —C(O)Me and —C(O)—CH═CH₂.

In one embodiment, the compounds of Formula I are those of Formula V:

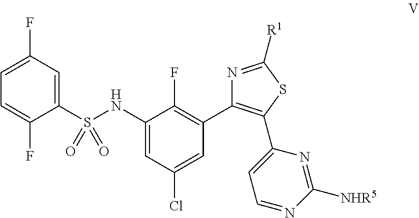

or pharmaceutically acceptable salts or solvates thereof,

wherein

R¹ and R⁵ are as defined above with respect to Formula I and any of its embodiments.

Preferred compounds of Formula V or pharmaceutically acceptable salts or solvates thereof are those wherein one or more of R¹ and R⁵ are defined as follows:

R¹ is selected from the group consisting C1-C6-alkyl and morpholinyl, said morpholinyl, being linked to the thiazol ring through a carbon atom and optionally substituted by C1-C6-alkyl, C3-C6-cycloalkyl or C1-C4-alkyloxycarbonyl; particularly R¹ is selected from the group consisting of C1-C4-alkyl and morpholinyl, said morpholinyl, being linked to the thiazol ring through a carbon atom and optionally substituted by C1-C4-alkyl, C3-C6-cycloalkyl or C1-C4-alkyloxycarbonyl; more particularly R¹ is selected from the group consisting of C1-C4-alkyl, morpholin-3-yl and morpholin-2-yl, said morpholin-3-yl and morpholin-2-yl being optionally substituted by C1-C4-alkyl, C3-C6-cycloalkyl or C1-C4-alkyloxycarbonyl; even more particularly R¹ is selected from the group consisting of C1-C4-alkyl and morpholin-3-yl, said morpholin-3-yl being optionally N-substituted by C3-C6-cycloalkyl or C1-C4-alkyloxycarbonyl; still more particularly R¹ is tert-butyl or morpholin-3-yl;

R⁵ is H.

In one embodiment, the compounds of Formula I are those of Formula VI:

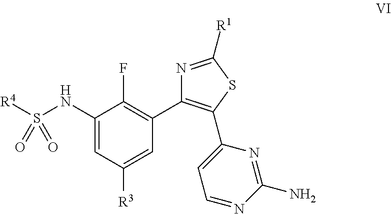

or pharmaceutically acceptable salts or solvates thereof,

wherein

R¹, R³ and R⁴ are as defined above with respect to Formula I and any of its embodiments.

Particularly preferred compounds of the invention are those listed in Table 1 hereafter:

TABLE 1

| Compound | Structure | Name |
|---|---|---|
| 1 | 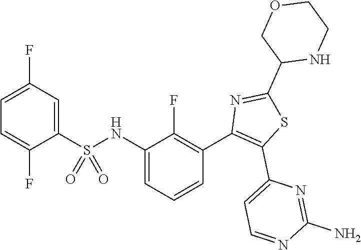 [SMILES: Nc1nccc(-c2sc(C3COCCN3)nc2-c2cccc(NS(=O)(=O)c3cc(F)ccc3F)c2F)n1] | N-{3-[5-(2-aminopyrimidin-4-yl)- 2-morpholin-3-yl-thiazol-4-yl]-2- fluorophenyl}-2,5- difluorobenzenesulfonamide |
| 2 | 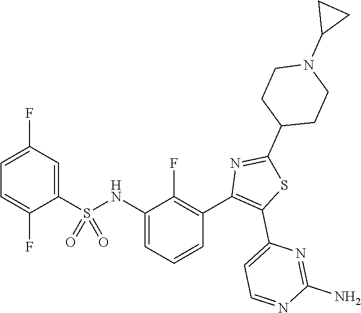 [SMILES: Nc1nccc(-c2sc(C3CCN(C4CC4)CC3)nc2-c2cccc(NS(=O)(=O)c3cc(F)ccc3F)c2F)n1] | N-{3-[5-(2-aminopyrimidin-4-yl)- 2-(1-cyclopropylpiperidin-4-yl)- thiazol-4-yl]-2-fluorophenyl}-2,5- difluorobenzenesulfonamide |
| 3 | 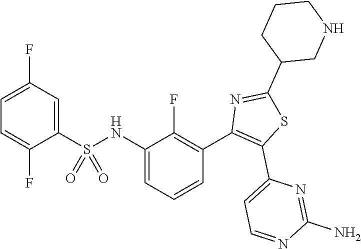 [SMILES: Nc1nccc(-c2sc(C3CCCNC3)nc2-c2cccc(NS(=O)(=O)c3cc(F)ccc3F)c2F)n1] | N-{3-[5-(2-aminopyrimidin-4-yl)- 2-piperidin-3-yl-thiazol-4-yl]-2- fluorophenyl}-2,5- difluorobenzenesulfonamide |
| 4 | 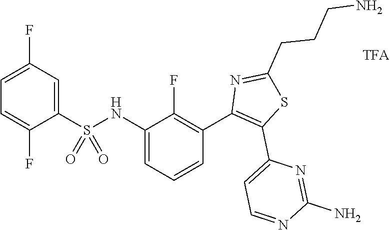 [SMILES: NCCCc1nc(-c2cccc(NS(=O)(=O)c3cc(F)ccc3F)c2F)c(-c2ccnc(N)n2)s1] | N-{3-[2-(3-aminopropyl)-5-(2- aminopyrimidin-4-yl)-thiazol-4- yl]-2-fluorophenyl}-2,5- difluorobenzenesulfonamide trifluoroacetate |
| 5 | 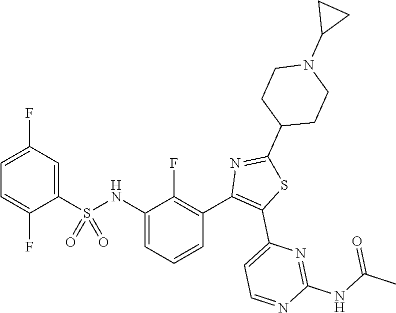 [SMILES: CC(=O)Nc1nccc(-c2sc(C3CCN(C4CC4)CC3)nc2-c2cccc(NS(=O)(=O)c3cc(F)ccc3F)c2F)n1] | N-(4-{2-(1-cyclopropylpiperidin- 4-yl)-4-[3-(2,5- difluorobenzenesulfonylamino)-2- fluorophenyl]-thiazol-5-yl}- pyrimidin-2-yl)-acetamide |
| 6 | 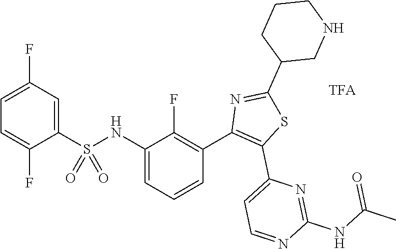 [SMILES: CC(=O)Nc1nccc(-c2sc(C3CCCNC3)nc2-c2cccc(NS(=O)(=O)c3cc(F)ccc3F)c2F)n1] | N-(4-{4-[3-(2,5- difluorobenzenesulfonylamino)-2- fluorophenyl]-2-piperidin-3-yl- thiazol-5-yl}-pyrimidin-2-yl)- acetamide trifluoroacetate |
| 7 | 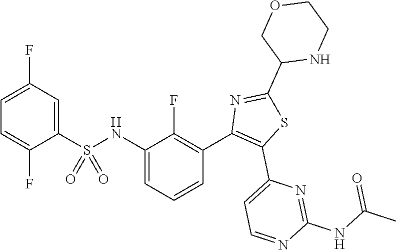 [SMILES: CC(=O)Nc1nccc(-c2sc(C3COCCN3)nc2-c2cccc(NS(=O)(=O)c3cc(F)ccc3F)c2F)n1] | N-(4-{4-[3-(2,5- difluorobenzenesulfonylamino)-2- fluorophenyl]-2-morpholin-3-yl- thiazol-5-yl}-pyrimidin-2-yl)- acetamide |
| 8 | 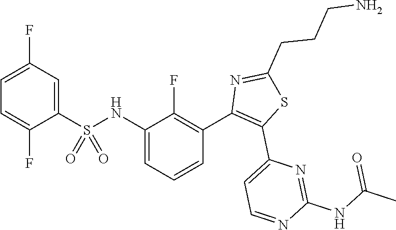 [SMILES: CC(=O)Nc1nccc(-c2sc(CCCN)nc2-c2cccc(NS(=O)(=O)c3cc(F)ccc3F)c2F)n1] | N-(4-{2-(3-aminopropyl)-4-[3- (2,5-difluorobenzene- sulfonylamino)-2-fluorophenyl]- thiazol-5-yl}-pyrimidin-2-yl)- acetamide |
| 9 | 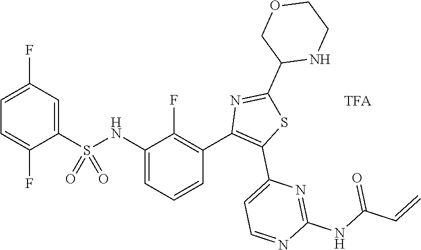 [SMILES: C=CC(=O)Nc1nccc(-c2sc(C3COCCN3)nc2-c2cccc(NS(=O)(=O)c3cc(F)ccc3F)c2F)n1] | N-(4-{4-[3-(2,5- difluorobenzenesulfonylamino)-2- fluorophenyl]-2-morpholin-3-yl- thiazol-5-yl}-pyrimidin-2-yl)- acrylamide trifluoroacetate |
| 10 | 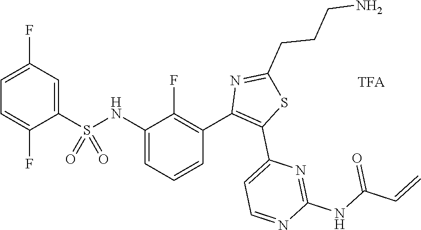 [SMILES: C=CC(=O)Nc1nccc(-c2sc(CCCN)nc2-c2cccc(NS(=O)(=O)c3cc(F)ccc3F)c2F)n1] | N-(4-{2-(3-aminopropyl)-4-[3- (2,5-difluorobenzene- sulfonylamino)-2-fluorophenyl]- thiazol-5-yl}-pyrimidin-2-yl)- acrylamide trifluoroacetate |
| 11 | 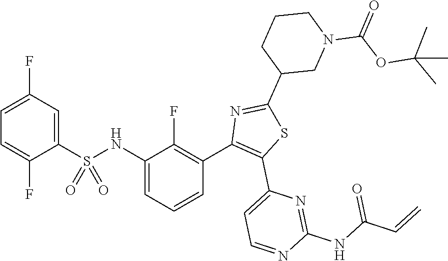 [SMILES: C=CC(=O)Nc1nccc(-c2sc(C3CCCN(C(=O)OC(C)(C)C)C3)nc2-c2cccc(NS(=O)(=O)c3cc(F)ccc3F)c2F)n1] | 3-{5-(2-acryloylaminopyrimidin- 4-yl)-4-[3-(2,5-difluoro- benzenesulfonylamino)-2- fluorophenyl]-thiazol-2-yl}- piperidine-1-carboxylic acid tert- butyl ester |
| 12 | 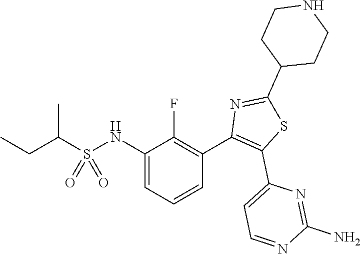 [SMILES: CCC(C)S(=O)(=O)Nc1cccc(-c2nc(C3CCNCC3)sc2-c2ccnc(N)n2)c1F] | butane-2-sulfonic acid {3-[5-(2- aminopyrimidin-4-yl)-2-piperidin- 4-yl-thiazol-4-yl]-2- fluorophenyl}-amide |
| 13 | 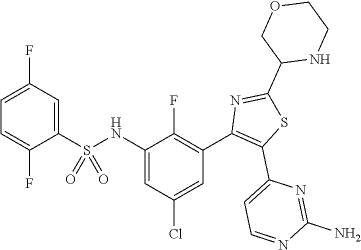 [SMILES: Nc1nccc(-c2sc(C3COCCN3)nc2-c2cc(Cl)cc(NS(=O)(=O)c3cc(F)ccc3F)c2F)n1] | N-{3-[5-(2-aminopyrimidin-4-yl)- 2-morpholin-3-yl-thiazol-4-yl]-5- chloro-2-fluorophenyl}-2,5- difluorobenzenesulfonamide |
| 14 | 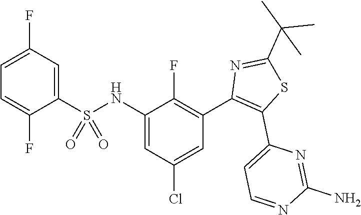 [SMILES: CC(C)(C)c1nc(-c2cc(Cl)cc(NS(=O)(=O)c3cc(F)ccc3F)c2F)c(-c2ccnc(N)n2)s1] | N-{3-[5-(2-aminopyrimidin-4-yl)- 2-tert-butyl-thiazol-4-yl]-5- chloro-2-fluorophenyl}-2,5- difluorobenzenesulfonamide |
| 15 | 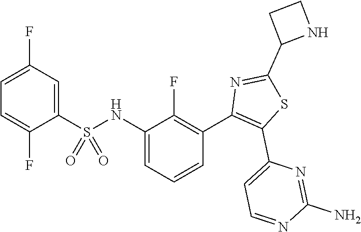 [SMILES: Nc1nccc(-c2sc(C3CCN3)nc2-c2cccc(NS(=O)(=O)c3cc(F)ccc3F)c2F)n1] | N-{3-[5-(2-aminopyrimidin-4-yl)- 2-azetidin-2-yl-thiazol-4-yl]-2- fluorophenyl}-2,5- difluorobenzenesulfonamide |
| 16 | 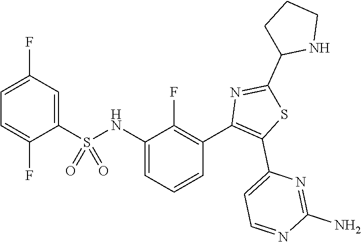 [SMILES: Nc1nccc(-c2sc(C3CCCN3)nc2-c2cccc(NS(=O)(=O)c3cc(F)ccc3F)c2F)n1] | N-{3-[5-(2-aminopyrimidin-4-yl)- 2-pyrrolidin-2-yl-thiazol-4-yl]-2- fluorophenyl}-2,5- difluorobenzenesulfonamide |
| 17 | 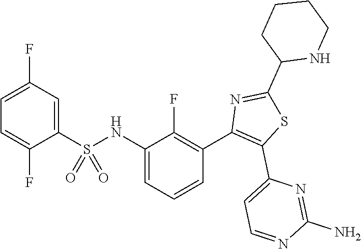 [SMILES: Nc1nccc(-c2sc(C3CCCCN3)nc2-c2cccc(NS(=O)(=O)c3cc(F)ccc3F)c2F)n1] | N-{3-[5-(2-aminopyrimidin-4-yl)- 2-piperidin-2-yl-thiazol-4-yl]-2- fluorophenyl}-2,5- difluorobenzenesulfonamide |
| 18 | 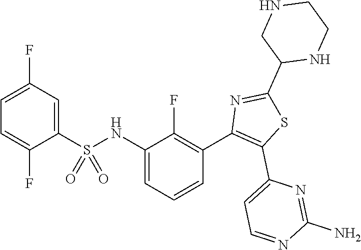 [SMILES: Nc1nccc(-c2sc(C3CNCCN3)nc2-c2cccc(NS(=O)(=O)c3cc(F)ccc3F)c2F)n1] | N-{3-[5-(2-aminopyrimidin-4-yl)- 2-piperazin-2-yl-thiazol-4-yl]-2- fluorophenyl}-2,5- difluorobenzenesulfonamide |
| 19 | 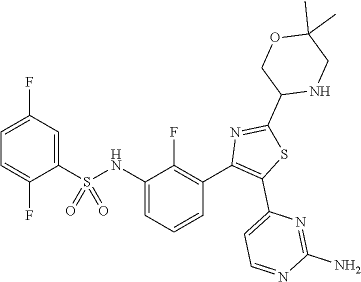 [SMILES: CC1(C)CNC(c2nc(-c3cccc(NS(=O)(=O)c4cc(F)ccc4F)c3F)c(-c3ccnc(N)n3)s2)CO1] | N-{3-[5-(2-aminopyrimidin-4-yl)- 2-(6,6-dimethylmorpholin-3- yl)thiazol-4-yl]-2-fluorophenyl}- 2,5-difluorobenzenesulfonamide |
| 20 | 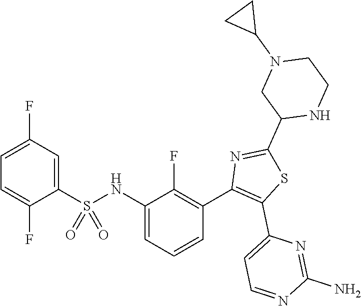 [SMILES: Nc1nccc(-c2sc(C3CN(C4CC4)CCN3)nc2-c2cccc(NS(=O)(=O)c3cc(F)ccc3F)c2F)n1] | N-{3-[5-(2-aminopyrimidin-4-yl)- 2-(4-cyclopropylpiperazin-2- yl)thiazol-4-yl]-2-fluorophenyl}- 2,5-difluorobenzenesulfonamide |

The compounds of the invention can be prepared by different ways with reactions known by the person skilled in the art. Reaction schemes as described in the example section illustrate by way of example different possible approaches.

The compounds of the invention are indeed modulators, in particular inhibitors, of protein kinases, particularly serine/threonine kinases, more particularly BRAF or mutants thereof. They further have the advantage of not activating PXR. The invention thus also provides the use of the compounds of the invention or pharmaceutically acceptable salts or solvates thereof as inhibitors of protein kinases, particularly serine/threonine kinases, more particularly BRAF or mutants thereof.

Accordingly, in a particularly preferred embodiment, the invention relates to the use of compounds of Formula I or any of its subformulae, in particular those of Table 1 above, or pharmaceutically acceptable salts or solvates thereof, as modulators, in particular inhibitors, of protein kinases, particularly serine/threonine kinases, more particularly BRAF or mutants thereof.

Applications

The inventors have demonstrated that the compounds of formula I or any of its subformulae, or pharmaceutically acceptable salts or solvates thereof, according to the present invention have the ability to modulate, in particular inhibit, protein kinases, particularly serine/threonine kinases, more particularly BRAF or mutants thereof, without activating the Pregnane X Receptor (PXR).

The compounds of the invention or pharmaceutically acceptable salts or solvates thereof are therefore useful in the treatment or prevention of diseases or disorders associated with abnormal or deregulated protein kinase activity. In other terms, the compounds of the invention or pharmaceutically acceptable salts or solvates thereof are therefore useful in the treatment or prevention of diseases or disorders mediated by protein kinase signalling.

The invention thus also relates to a compound of the invention or a pharmaceutically acceptable salt or solvate thereof for use in treating or preventing a disease or disorder associated with deregulated protein kinase activity.

In one embodiment, the invention relates to a compound of the invention or a pharmaceutically acceptable salt or solvate thereof for use in treating or preventing a disease or disorder associated with deregulated protein kinase activity, wherein the protein kinase is selected from tyrosine kinases, serine/threonine kinases and kinases with dual specificity, particularly wherein the protein kinase is selected from RAF family, EGFR family, ALK, MEK, FGFR1, FGFR2, FGFR3, FGFR4, FLT3, IGF1R, c-Met, JAK family, PDGFR α and β, RET, AXL, c-KIT, TrkA, TrkB, TrkC, ROS1, BTK and Syk, more particularly wherein the protein kinase is selected from A-RAF, B-RAF and C-RAF, still more particularly wherein the protein kinase is B-RAF or mutant forms thereof, even more particularly wherein the protein kinase is B-RAF or mutant forms thereof, wherein the mutant forms are selected from R461I, I462S, G463E, G463V, G465A, G465E, G465V, G468A, G468E, N580S, E585K, D593V, F594L, G595R, L596V, T598I, V599D, V599E, V599K, V599R, V600E and A727V, particularly V599E and V600E, more particularly V599E.

Diseases associated with deregulated protein kinase activity within the meaning of the present invention include, but are not limited to, cancer, in particular cancer selected from the group consisting of melanoma, lung cancer, colorectal cancer, gastro-intestinal stromal cancer and pancreatic cancer.

Thus, there is provided a compound of the invention or a pharmaceutically acceptable salt or solvate thereof for use in treating or preventing cancer. In particular, there is provided a compound of the invention or a pharmaceutically acceptable salt or solvate thereof for use in treating or preventing a cancer selected from the group consisting of melanoma, lung cancer, colorectal cancer, gastro-intestinal stromal cancer and pancreatic cancer. More particularly, there is provided a compound of the invention or a pharmaceutically acceptable salt or solvate thereof for use in treating or preventing a cancer selected from the group consisting of melanoma, lung cancer, colorectal cancer and gastro-intestinal stromal cancer.

In one embodiment, there is provided a compound of the invention or a pharmaceutically acceptable salt or solvate thereof for use in treating or preventing melanoma, in particular metastatic melanoma.

In one embodiment, there is provided a compound of the invention or a pharmaceutically acceptable salt or solvate thereof for use in treating or preventing lung cancer, in particular small cell lung carcinoma (SCLC), non-small cell lung carcinoma (NSCLC) and lung adenocarcinoma.

In one embodiment, there is provided a compound of the invention or a pharmaceutically acceptable salt or solvate thereof for use in treating or preventing colorectal cancer.

In one embodiment, there is provided a compound of the invention or a pharmaceutically acceptable salt or solvate thereof for use in treating or preventing gastro-intestinal stromal cancer.

In one embodiment, there is provided a compound of the invention or a pharmaceutically acceptable salt or solvate thereof for use in treating or preventing pancreatic cancer, in particular pancreatic neuroendocrine cancer.

In other terms, the invention also relates to a method of treating or preventing a disease or disorder associated with deregulated protein kinase activity, comprising the administration of a therapeutically effective amount of a compound of the invention or a pharmaceutically acceptable salt or solvate thereof, to a patient in need thereof. Preferably the patient is a warm-blooded animal, more preferably a human. The diseases or disorders associated with deregulated protein kinase activity are preferably those defined above.

The invention also relates to a method of treating or preventing cancer, comprising the administration of a therapeutically effective amount of a compound of the invention or pharmaceutically acceptable salt or solvate thereof, to a patient in need thereof. Preferably the patient is a warm-blooded animal, more preferably a human. In particular, the invention relates to a method of treating or preventing a cancer selected from the group consisting of melanoma, lung cancer, colorectal cancer, gastro-intestinal stromal cancer and pancreatic cancer, comprising the administration of a therapeutically effective amount of a compound of the invention or pharmaceutically acceptable salt or solvate thereof, to a patient in need thereof.

The invention further provides the use of a compound of the invention or a pharmaceutically acceptable salt or solvates thereof for the manufacture of a medicament for use in treating or preventing a disease or disorder associated with deregulated protein kinase activity. Preferably the patient is a warm-blooded animal, more preferably a human. The diseases or disorders associated with deregulated protein kinase activity are preferably those defined above.

The invention further provides the use of a compound of the invention or a pharmaceutically acceptable salt or solvates thereof for the manufacture of a medicament for use in treating or preventing cancer. Preferably the patient is a warm-blooded animal, more preferably a human. In particular, the invention further provides the use of a compound of the invention or a pharmaceutically acceptable salt or solvate thereof for the manufacture of a medicament for use in treating or preventing a cancer selected from the group consisting of melanoma, lung cancer, colorectal cancer, gastro-intestinal stromal cancer and pancreatic cancer.

According to a further feature of the present invention, there is provided a compound of the invention or a pharmaceutically acceptable salt or solvate thereof for use in modulating, in particular inhibiting, a protein kinase, particularly a serine/threonine kinase, more particularly BRAF or mutants thereof, in a patient in need of such treatment, comprising administering to said patient an effective amount of a compound of the present invention, or a pharmaceutically acceptable salt or solvate thereof. In other terms, the invention also provides a method for modulating, in particular inhibiting, a protein kinase, particularly a serine/threonine kinase, more particularly BRAF or mutants thereof, in a patient in need of such treatment, which comprises administering to said patient an effective amount of a compound of the present invention, or a pharmaceutically acceptable salt or solvate thereof. Preferably, the patient is a warm blooded animal, and even more preferably a human.

According to the present invention, the compound of the invention may be administered as a pharmaceutical formulation in a therapeutically effective amount by any of the accepted modes of administration, preferably by intravenous or oral route.

Therapeutically effective amount ranges are typically from 0.1 to 50 000 μg/kg of body weight daily, preferably from 1 000 to 40 000 μg/kg of body weight daily, depending upon numerous factors such as the severity of the disease to be treated, the age and relative health of the subject, the potency of the compound, the route and the form of administration, the indication towards which the administration is directed, and the preferences and experience of the medical practitioner involved. One of ordinary skill in the art of treating such diseases will be able in reliance upon personal knowledge, to ascertain a therapeutically effective amount of the antineoplastic agent of the present invention for a given cancer.

According to one embodiment, the compounds of the invention, their pharmaceutical acceptable salts or solvates may be administered as part of a combination therapy. Thus, are included within the scope of the present invention embodiments comprising co-administration of, and compositions and medicaments which contain, in addition to a compound of the present invention, a pharmaceutically acceptable salt or solvate thereof as active ingredient, additional therapeutic agents and/or active ingredients. Such multiple drug regimens, often referred to as combination therapy, may be used in the treatment or prevention of any disease or disorder associated with deregulated protein kinase activity, particularly those defined above.

Thus, the methods of treatment and pharmaceutical compositions of the present invention may employ the compounds of the invention or their pharmaceutical acceptable salts or solvates thereof in the form of monotherapy, but said methods and compositions may also be used in the form of multiple therapy in which one or more compounds of Formula I or their pharmaceutically acceptable salts or solvates are co-administered in combination with one or more other therapeutic agents. Additional therapeutic agents include, but are not limited to, other anticancer agents, pain medication, antidepressant or anti-inflammatory agents.

The invention also provides pharmaceutical compositions comprising a compound of the invention or a pharmaceutically acceptable salt or solvate thereof and at least one pharmaceutically acceptable excipient. As indicated above, the invention also covers pharmaceutical compositions which contain, in addition to a compound of the present invention, a pharmaceutically acceptable salt or solvate thereof as active ingredient, additional therapeutic agents and/or active ingredients.

The invention also provides a compound of the invention or a pharmaceutically acceptable salt or solvate thereof for use in a therapeutic treatment in humans or animals.

Another object of this invention is a medicament comprising at least one compound of the invention, or a pharmaceutically acceptable salt or solvate thereof, as active ingredient.

Generally, for pharmaceutical use, the compounds of the invention may be formulated as a pharmaceutical preparation comprising at least one compound of the invention and at least one pharmaceutically acceptable excipient, and optionally one or more further pharmaceutically active compounds.

By means of non-limiting examples, such a formulation may be in a form suitable for oral administration (e.g. as a tablet, capsule, or as an ingestible solution), for parenteral administration (such as by intravenous, intramuscular or subcutaneous injection or intravenous infusion), for topical administration (including ocular), intracerebral administration, sublingual administration, aerosol administration, for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

For example, the compound of the invention or a pharmaceutical composition comprising a compound of the invention can be administered orally in the form of tablets, coated tablets, pills, capsules, soft gelatin capsules, oral powders, granules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, a disintegrant such as starch (preferably corn, potato or tapioca starch), sodium starch glycollate, croscarmellose sodium and certain complex silicates, a binder such as polyvinylpyrrolidone, hydroxypropylmethylcellulose (HPMC), hydroxypropylcellulose (HPC), sucrose, gelatin and acacia, a lubricant such as magnesium stearate, stearic acid, glyceryl behenate. Solid compositions of a similar type may also be employed as fillers in hard gelatin capsules. Preferred excipients in this regard include lactose, saccharose, sorbitol, mannitol, potato starch, corn starch, amylopectin, cellulose derivatives or gelatin. Hard gelatin capsules may contain granules of the compound of the invention.

Soft gelatin capsules may be prepared with capsules containing the compound of the invention, vegetable oil, waxes, fat, or other suitable vehicle for soft gelatin capsules. As an example, the acceptable vehicle can be an oleaginous vehicle, such as a long chain triglyceride vegetable oil (e.g. corn oil).

Dispersible powders and granules suitable for preparation of an aqueous suspension by the addition of water may contain the active ingredient in a mixture with dispersing agents, wetting agents, and suspending agents and one or more preservatives. Additional excipients, for example sweetening, flavouring and colouring agents, may also be present. These compositions may be preserved by the addition of an anti-oxidant such as ascorbic acid.

Liquid dosage forms for oral administration may include pharmaceutically acceptable, solutions, emulsions, suspensions, syrups, and elixirs containing inert diluents commonly used in the art, such as water or an oleaginous vehicle. Liquid dosage form may be presented as a dry product for constitution with water or other suitable vehicle before use. Such compositions may also comprise adjuvants, such as wetting agents, emulsifying and suspending agents, complexing agents such as 2-hydroxypropyl-beta-cyclodextrin, sulfobutylether-beta-cylodextrin, and sweetening, flavouring, perfuming agents, colouring matter or dyes with diluents such as water, ethanol, propylene glycol and glycerin, and combinations thereof. These compositions may be preserved by the addition of an anti-oxidant such as butylated hydroxyanisol or alpha-tocopherol.

Finely divided powder of the compound of the invention may be prepared for example by micronisation or by processes known in the art. The compound of the invention may be milled using known milling procedures such as wet milling to obtain a particle size appropriate for tablet formation and for other formulation types.

If the compound of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent; and/or by using infusion techniques.

The compound of the invention can be administered via the parenteral route with a ready available or a depot-type formulation.

The pharmaceutical compositions for the parenteral administration of a ready available formulation may be in the form of a sterile injectable aqueous or oleagenous solution or suspension in a non-toxic parenterally-acceptable diluent or solvent and may contain formulatory agents such as suspending, stabilising dispersing, wetting and/or complexing agents such as cyclodextrin e.g. 2-hydroxypropyl-beta-cyclodextrin, sulfobutylether-beta-cylodextrin.

The depot-type formulation for the parenteral administration may be prepared by conventional techniques with pharmaceutically acceptable excipient including without being limited to, biocompatible and biodegradable polymers (e.g. poly(β-caprolactone), poly(ethylene oxide), poly(glycolic acid), poly[(lactic acid)-co-(glycolic acid) . . . )], poly(lactic acid) . . . ), non-biodegradable polymers (e.g. ethylene vinylacetate copolymer, polyurethane, polyester(amide), polyvinyl chloride . . . ) aqueous and non-aqueous vehicles (e.g. water, sesame oil, cottonseed oil, soybean oil, castor oil, almond oil, oily esters, ethyl alcohol or fractionated vegetable oils, propylene glycol, DMSO, THF, 2-pyrrolidone, N-methylpyrrolidinone, N-vinylpyrrolidinone . . . ).

Alternatively, the active ingredient may be in dry form such as a powder, crystalline or freeze-dried solid for constitution with a suitable vehicle. The preparation of suitable parenteral formulations under sterile conditions is readily accomplished by standard pharmaceutical techniques well known to those skilled in the art.

As indicated, the compound of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, (for example from Ineos Fluor), carbon dioxide or other suitable gas. In the case of a pressurised aerosol, the dosage unit may be determined by providing a valve to deliver a metered amount. The pressurised container, pump, spray or nebuliser may contain a solution or suspension of the active compound. Capsules and cartridges (made, for example, from gelatin) for use in an inhaler or insufflator may be formulated to contain a powder mix of the compound and a suitable powder base such as lactose or starch. For compositions suitable and/or adapted for inhaled administration, it is preferred that the compound or salt of formula (I) is in a particle-size-reduced form, and more preferably the size-reduced form is obtained or obtainable by micronisation. The preferable particle size of the size-reduced (e.g. micronised) compound or salt or solvate is defined by a D50 value of about 0.5 to about 50 microns (for example as measured using laser diffraction).

Alternatively, the compound of the present invention can be administered in the form of a suppository or pessary, or it may be applied topically in the form of a gel, hydrogel, lotion, solution, cream, ointment or dusting powder. The compound of the present invention may also be dermally or transdermally administered, for example, by the use of a skin patch. They may also be administered by the pulmonary or rectal routes. It may also be administered by the ocular route. For ophthalmic use, the compound can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, it may be formulated in an ointment such as petrolatum.

For topical application to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene, polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

### Definitions

The definitions and explanations below are for the terms as used throughout the entire application, including both the specification and the claims.

Unless otherwise stated, any reference to compounds of the invention herein, means the compounds as such as well as their pharmaceutically acceptable salts and solvates.

When describing the compounds of the invention, the terms used are to be construed in accordance with the following definitions, unless indicated otherwise.

The term “unsubstituted” as used herein means that a radical, a group or a residue carries no substituent. The term “substituted” means that a radical, a group or a residue carries one or more substituents. The term “N-substituted” means that the one or more substituents are carried on a N atom of the radical, group or residue.

The term “halo” or “halogen” refers to the atoms of the group 17 of the periodic table (halogens) and includes in particular fluorine (F), chlorine (Cl), bromine (Br) and iodine (I) atom. Preferred halo groups are fluoro (F) and chloro (Cl), fluoro being particularly preferred.

The term “alkyl” by itself or as part of another substituent refers to a hydrocarbyl radical of Formula CₙH₂ₙ₊₁ wherein n is a number greater than or equal to 1. Alkyl groups may thus comprise 1 or more carbon atoms and generally, according to this invention comprise from 1 to 12, more preferably from 1 to 8 carbon atoms, and still more preferably from 1 to 6 carbon atoms. Alkyl groups within the meaning of the invention may be linear or branched. Examples of alkyl groups include but are not limited to methyl, ethyl, propyl, isopropyl, butyl, sec-butyl, isobutyl, tert-butyl, pentyl, sec-pentyl, isopentyl, hexyl and isohexyl.

The term “alkenyl” by itself or as part of another substituent refers to a hydrocarbyl radical at least one double carbon-carbon bond. Alkenyl groups may thus comprise 2 or more carbon atoms and generally, according to this invention comprise from 2 to 12, more preferably from 2 to 8 carbon atoms, and still more preferably from 2 to 6 carbon atoms.

The term “alkynyl” by itself or as part of another substituent refers to a hydrocarbyl radical comprising at least one triple carbon-carbon bond. Alkynyl groups may thus comprise 2 or more carbon atoms and generally, according to this invention comprise from 2 to 12, more preferably from 2 to 8 carbon atoms, and still more preferably from 2 to 6 carbon atoms.

The term “alkoxy” by itself or as part of another substituent refers to a —O-alkyl group, wherein alkyl is as defined above. Examples of alkoxy groups include but are not limited to methoxy, ethoxy, propoxy, isoproxy, butoxy, sec-butoxy, isobutoxy, tert-butoxy, pentoxy, sec-pentoxy and isopentoxy.

The term “aminoalkyl” by itself or as part of another substituent refers to a -alkyl-NH₂ group, wherein alkyl is as defined above.

The term “alkyloxycarbonyl” by itself or as part of another substituent refers to a —C(O)—O-alkyl group, wherein alkyl is as defined above.

The term “haloalkyl” or “halogenoalkyl” alone or in combination, refers to an alkyl radical having the meaning as defined above wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl and the like.

The term “cycloalkyl” as used herein is a monovalent, saturated, or unsaturated monocyclic or bicyclic hydrocarbyl group. Cycloalkyl groups may comprise 3 or more carbon atoms in the ring and generally, according to this invention comprise from 3 to 10, more preferably from 3 to 8 carbon atoms, and still more preferably from 3 to 6 carbon atoms. Examples of cycloalkyl groups include but are not limited to cyclopropyl, cyclobutyl, cyclopentyl, cyclohexyl and cycloheptyl.

The term “heteroatom” as used herein refers to any atom that is not carbon or hydrogen. Non-limiting examples of such heteroatoms include nitrogen, oxygen, sulfur, and phosphorus. Preferred heteroatoms according to the invention are nitrogen, oxygen and sulfur.

The terms “heterocyclyl”, “heterocycloalkyl” or “heterocyclo” as used herein by itself or as part of another group refer to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen, oxygen and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. Examples of heterocyclyl groups include but are not limited to aziridinyl, azetidinyl, pyrrolidinyl, piperidinyl, azepanyl, piperazinyl, morpholinyl. Preferred heterocyclyl groups according to the invention are azetidinyl, pyrrolidinyl, piperidinyl, piperazinyl and morpholinyl.

The term “aryl” as used herein refers to a polyunsaturated, aromatic hydrocarbyl group having a single ring (i.e. phenyl) or multiple aromatic rings fused together (e.g. naphthyl), typically containing 5 to 12 atoms; preferably 6 to 10, wherein at least one ring is aromatic. Examples of aryl groups include but are not limited to phenyl, biphenyl, 1-naphthyl (or naphthalene-1-yl), 2-naphthyl (or naphthalene-2-yl), anthracenyl, indanyl, indenyl, 1,2,3,4-tetrahydronaphthyl. Preferred aryl group according to the invention is phenyl.

The term “heteroaryl” as used herein by itself or as part of another group refers but is not limited to 5 to 12 carbon-atom aromatic rings or ring systems containing 1 to 2 rings which are fused together, typically containing 5 to 6 atoms; at least one of which is aromatic, in which one or more carbon atoms in one or more of these rings is replaced by oxygen, nitrogen and/or sulfur atoms where the nitrogen and sulfur heteroatoms may optionally be oxidized and the nitrogen heteroatoms may optionally be quaternized. Examples of heteroaryl groups include but are not limited to pyridyl, pyrazinyl, pyrimidinyl, pyridazinyl, triazinyl, quinolinyl, quinoxalinyl, quinazolinyl, furanyl, benzofuranyl, pyrrolyl, indolyl, thiophenyl, benzothiophenyl, imidazolyl, benzimidazolyl, pyrazolyl, indazolyl, oxazolyl, benzoxazolyl, isoxazolyl, benzisoxazolyl, thiazolyl, and benzothiazolyl, triazolyl, oxadiazolyl, thiadiazolyl, dioxazolyl, dithiazolyl and tetrazolyl. Preferred heteroaryl group according to the invention is thiazolyl.

The term “haloaryl” or “halogenoaryl” alone or in combination, refers to an aryl radical having the meaning as defined above wherein one or more hydrogens are replaced with a halogen as defined above.

The compounds of the invention containing a basic functional group may be in the form of pharmaceutically acceptable salts. Pharmaceutically acceptable salts of the compounds of the invention containing one or more basic functional groups include in particular the acid addition salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, cinnamate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Pharmaceutically acceptable salts of compounds of Formula I and subformulae may for example be prepared as follows:

(i) reacting the compound of Formula I or any of its subformulae with the desired acid; or

(ii) converting one salt of the compound of Formula I or any of its subformulae to another by reaction with an appropriate acid or by means of a suitable ion exchange column.

All these reactions are typically carried out in solution. The salt, may precipitate from solution and be collected by filtration or may be recovered by evaporation of the solvent. The degree of ionization in the salt may vary from completely ionized to almost non-ionized.

The term “solvate” is used herein to describe a molecular complex comprising the compound of the invention and one or more pharmaceutically acceptable solvent molecules, for example, ethanol. The term “hydrate” is employed when said solvent is water.

The compounds of the invention include compounds of the invention as hereinbefore defined, including all polymorphs and crystal habits thereof, prodrugs and isomers thereof (including optical, geometric and tautomeric isomers) and isotopically-labeled compounds of the invention.

In addition, although generally, with respect to the salts of the compounds of the invention, pharmaceutically acceptable salts are preferred, it should be noted that the invention in its broadest sense also includes non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of the invention.

The term “patient” refers to a warm-blooded animal, more preferably a human, who/which is awaiting or receiving medical care or is or will be the object of a medical procedure.

The term “human” refers to subjects of both genders and at any stage of development (i.e. neonate, infant, juvenile, adolescent, adult). In one embodiment, the human is an adolescent or adult, preferably an adult.

The terms “treat”, “treating” and “treatment”, as used herein, are meant to include alleviating or abrogating a condition or disease and/or its attendant symptoms.

The term “therapeutically effective amount” (or more simply an “effective amount” or “suitable dose”) as used herein means the amount of active agent or active ingredient which is sufficient to achieve the desired therapeutic or prophylactic effect in the individual to which it is administered.

The term “administration”, or a variant thereof (e.g., “administering”), means providing the active agent or active ingredient, alone or as part of a pharmaceutically acceptable composition, to the patient in whom/which the condition, symptom, or disease is to be treated.

By “pharmaceutically acceptable” is meant that the ingredients of a pharmaceutical composition are compatible with each other and not deleterious to the patient thereof.

The term “excipient” as used herein means a substance formulated alongside the active agent or active ingredient in a pharmaceutical composition or medicament. Acceptable excipients for therapeutic use are well known in the pharmaceutical art, and are described, for example, in Remington's Pharmaceutical Sciences, 21^{st} Edition 2011. The choice of excipient can be selected with regard to the intended route of administration and standard pharmaceutical practice. The excipient must be acceptable in the sense of being not deleterious to the recipient thereof. The at least one pharmaceutically acceptable excipient may be for example, a binder, a diluent, a carrier, a lubricant, a disintegrator, a wetting agent, a dispersing agent, a suspending agent, and the like.

The term “cancer” as used herein refers to the physiological condition in subjects that is characterized by unregulated or dysregulated cell growth or death. The term “cancer” includes solid tumors and blood born tumors, whether malignant or benign.

Examples of cancer include, but are not limited to:

Acinar adenocarcinoma, acinar carcinoma, acral-lentiginous melanoma, actinic keratosis, adenocarcinoma, adenocystic carcinoma, adenosquamous carcinoma, adnexal carcinoma, adrenal rest tumor, adrenocortical carcinoma, aldosterone secreting carcinoma, alveolar soft part sarcoma, amelanotic melanoma, ameloblastic thyroid carcinoma, angiosarcoma, apocrine carcinoma, Askin's tumor, astrocytoma, basal cell carcinoma, basaloid carcinoma, basosquamous cell carcinoma, biliary cancer, bone cancer, bone marrow cancer, botryoid sarcoma, brain cancer, breast cancer, bronchioalveolar carcinoma, bronchogenic adenocarcinoma, bronchogenic carcinoma, carcinoma ex pleomorphic adenoma, cervical cancer, chloroma, cholangiocellular carcinoma, chondrosarcoma, choriocarcinoma, choroid plexus carcinoma, clear cell adenocarcinoma, colon cancer, colorectal cancer, comedocarcinoma, cortisol-producing carcinoma, cylindrical cell carcinoma, dedifferentiated liposarcoma, ductal adenocarcinoma of the prostate, ductal carcinoma, ductal carcinoma in situ, duodenal cancer, eccrine carcinoma, embryonal carcinoma, endometrial carcinoma, endometrial stromal carcinoma, epithelioid sarcoma, esophageal cancer, Ewing's sarcoma, exophytic carcinoma, fibroblastic sarcoma, fibrocarcinoma, fibrolamellar carcinoma, fibrosarcoma, follicular thyroid carcinoma, gallbladder cancer, gastric adenocarcinoma, gastro-intestinal stromal cancer, giant cell carcinoma, giant cell sarcoma, giant cell tumor of bone, glioma, glioblastoma or glioblastoma multiforme, granulose cell carcinoma, head & neck cancer, hemangioma, hemangiosarcoma, hepatoblastoma, hepatocellular carcinoma, Hürthle cell carcinoma, ileal cancer, infiltrating lobular carcinoma, inflammatory carcinoma of the breast, intraductal carcinoma, intraepidermal carcinoma, jejuna cancer, Kaposi's sarcoma, Krukenberg's tumor, Kulchitsky cell carcinoma, Kupffer cell sarcoma, large cell carcinoma, larynx cancer, lentigo maligna melanoma, liposarcoma, liver cancer, lobular carcinoma, lobular carcinoma in situ, lung cancer, lymphoepithelioma, lymphoepithelioma, lymphosarcoma, malignant melanoma, medullary carcinoma, medullary thyroid carcinoma, medulloblastoma, meningeal carcinoma, Merkel cell carcinoma, micropapillary carcinoma, mixed cell sarcoma, mucinous carcinoma, mucoepidermoid carcinoma, mucosal melanoma, myxoid liposarcoma, myxosarcoma, nasopharyngeal carcinoma, nephroblastoma, neuroblastoma, nodular melanoma, non-clear cell renal cancer, non-small cell lung cancer, oat cell carcinoma, ocular melanoma, oral cancer, osteoid carcinoma, osteosarcoma, ovarian cancer, Paget's carcinoma, pancreatic cancer, pancreatoblastoma, papillary adenocarcinoma, papillary carcinoma, papillary thyroid carcinoma, pelvic cancer, periampullary carcinoma, phyllodes tumor, pituitary cancer, pleomorphic liposarcoma, pleuropulmonary blastoma, primary intraosseous carcinoma, prostate cancer, rectal cancer, renal cell carcinoma, retinoblastoma, rhabdomyosarcoma, round cell liposarcoma, scar cancer, schistosomal bladder cancer, schneiderian carcinoma, sebaceous carcinoma, signet-ring cell carcinoma, skin cancer, small cell lung cancer, small cell osteosarcoma, soft tissue sarcoma, splindle cell carcinoma, spindle cell sarcoma, squamous cell carcinoma, stomach cancer, superficial spreading melanoma, synovial sarcoma, telangiectatic sarcoma, terminal duct carcinoma, testicular cancer, thyroid cancer, transitional cell carcinoma, tubular carcinoma, tumorigenic melanoma, undifferentiated carcinoma, urachal adenocarcinoma, urinary bladder cancer, uterine cancer, uterine corpus carcinoma, uveal melanoma, aginal cancer, cerrucous carcinoma, villous carcinoma, well-differentiated liposarcoma, Wilm's tumor or yolk sac tumor. Preferred cancers according to the invention are melanoma, lung cancer, colorectal cancer, gastro-intestinal stromal cancer and pancreatic cancer.

The terms “anticancer agent”, “anticancer drug”, “chemotherapeutic agent” or “cytotoxic agent”, as used herein, refer to a chemical agent used to treat or prevent cancer, administered in regimens of one or more cycles, alone or combined with one or more agents over a period of days to weeks. Such agents are toxic to cells with high proliferative rates, such as cancer cells.

The present invention will be better understood with reference to the following examples. These examples are intended to representative of specific embodiments of the invention, and are not intended as limiting the scope of the invention.

### Examples

Abbreviations

In the context of the present invention, the following abbreviations and empirical formulae are used:

Boc: tert-butyloxycarbonyl

C18 column: reversed-phase C18 column

° C.: degree Celsius

g: gram(s)

h: hour(s)

HATU: Hexafluorophosphate Azabenzotriazole Tetramethyl Uronium

HPLC: high performance liquid chromatography

LC/MS: liquid chromatography/mass spectrometry

M: mole(s) per liter

mg: milligram(s)

MH⁺: pseudo-molecular ion (positive ion mode in mass spectrometry)

MHz: megahertz

μL: microliter(s)

mL milliliter(s)

mmol: millimole(s)

mol: mole(s)

NMR: nuclear Magnetic Resonance

Other features, properties and advantages of the invention will emerge more clearly from the description and examples that follow.

Equipment and Analytical Methods Used for the Syntheses of Examples

Microwaves irradiation:

Apparatus: CEM Discover with Synergy Software.

Method: 10 or 30 mL sealed tube, power 50 W, high stirring, and irradiation time 15 or 30 min.

Flash chromatography:

Apparatus: Biotage SP with auto-collector and UV detection (2 wavelengths).

Normal phase columns: 10, 30 or 100 g Biotage external dry load cartridge kit, packed with Sigma-Aldrich 40-63 μm silica gel.

Reverse phase column: 30, 120 g Biotage SNAP Cartridges, KP-C18-HS.

Liquid Chromatography:

Apparatus: Waters alliance 2695 HPLC system with autosampler and Waters 2996 diode array detector.

Analytical method:
Column: Macherey-Nagel Nucleoshell RP18 plus (5 μm, 4 mm×100 mm).
Column temperature: 40° C.
Solvents: A (H₂O 99.9%, H₂CO₂ 0.1%); B (CH₃CN 99.9%, H₂CO₂ 0.1%).
Flow rate: 1 mL/min.
Gradient (A/B v/v): 90/10 (t=0 min), 90/10 (t=1 min), 0/100 (t=7 min), 0/100 (t=10 min).
Detection: 210-400 nm range.

Mass Spectrometer:

Apparatus: Waters Micromass ZQ (simple quad).

Mass detection method: Electrospray positive mode (ESI+), mass range: 50-800 uma.

NMR Spectrometer:

Apparatus: Bruker 400 MHz.

Methods: ¹H NMR spectra performed in DMSO-d6 using DMSO-d5 as internal reference, chemical shifts expressed in parts per million (ppm), signals expressed as follows: s=singlet, d=doublet, t=triplet, q=quadruplet, sept=septuplet, dd=double doublet, dt=double triplet, m=multiplet or large singlet, br=broad, H=proton.

### Example 1: N-{3-[5-(2-Aminopyrimidin-4-yl)-2-morpholin-3-yl-thiazol-4-yl]-2-fluorophenyl}-2,5-difluorobenzenesulfonamide

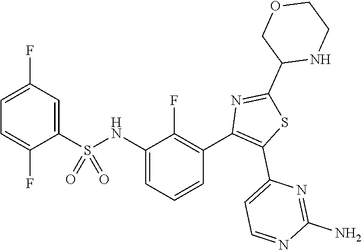 [SMILES: Nc1nccc(-c2sc(C3COCCN3)nc2-c2cccc(NS(=O)(=O)c3cc(F)ccc3F)c2F)n1]

Step 1: 3-Amino-2-fluorobenzoic acid methyl ester

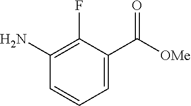 [SMILES: COC(=O)c1cccc(N)c1F]

5.00 g (32.2 mmol) of 3-amino-2-fluorobenzoic acid are dissolved in 50 mL of anhydrous methanol under argon. 2.47 mL (33.8 mmol) of thionyl chloride are slowly added at 0° C., and the reaction is then refuxed for 4 h. The solution is cooled to room temperature. Solvent is removed under reduced pressure. Reaction mixture is quenched with a saturated solution of sodium bicarbonate and extracted 3 times with ethyl acetate. Combined organic layers are dried over anhydrous sodium sulfate and filtered. The filtrate is concentrated under reduced pressure and the crude product is purified by flash chromatography through a 100 g silica gel column using a dichloromethane/methanol mixture as eluent. 5.03 g of the title compound are obtained.

Yield: 92%.

MH⁺: 170.3.

Step 2: 3-(2,5-Difluorobenzenesulfonylamino)-2-fluorobenzoic acid methyl ester

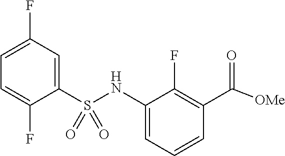 [SMILES: COC(=O)c1cccc(NS(=O)(=O)c2cc(F)ccc2F)c1F]

To a solution of 5.03 g (29.7 mmol) of 3-amino-2-fluorobenzoic acid methyl ester (described in the previous step) in 50 mL of anhydrous pyridine, are added, under argon, 4.8 mL (35.7 mmol) of 2,5-difluorobenzenesulfonyl chloride at 0° C. The mixture is stirred at 0° C. for 20 min, then at room temperature overnight. After the complete conversion, the reactional mixture is concentrated under reduced pressure, dissolved in dichloromethane, washed 4 times with hydrochloric acid 0.5N and once with brine. The organic layer is dried over anhydrous sodium sulfate and filtered. The filtrate is concentrated under reduced pressure and crude product is purified by flash chromatography on a 100 g silica gel column using a dichloromethane/methanol mixture as eluent. 8.14 g of the title compound are obtained.

Yield: 80%.

MH⁺: 346.5.

Step 3: N-{3-[2-(2-Chloropyrimidin-4-yl)-acetyl]-2-fluoro-phenyl}-2,5-difluorobenzenesulfonamide

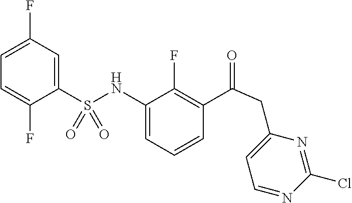 [SMILES: O=C(Cc1ccnc(Cl)n1)c1cccc(NS(=O)(=O)c2cc(F)ccc2F)c1F]

To a solution of 4 g (11.6 mmol) of 3-(2,5-difluorobenzenesulfonylamino)-2-fluorobenzoic acid methyl ester (described in the previous step) in 40 mL of anhydrous tetrahydrofuran, are slowly added at −15° C. under argon, to 40.5 mL (40.5 mmol) of a solution of lithium bis(trimethylsilyl)amide (1 M in tetrahydrofuran). The reaction is stirred at −15° C. for 20 min then a solution of 1.79 g (13.8 mmol) of 2-chloro-4-methylpyrimidine in 10 mL of anhydrous tetrahydrofuran is slowly added while keeping the bath temperature between −20 and −15° C. The reaction is stirred at this temperature for 30 more min, then 15 mL of a saturated solution of ammonium chloride is slowly added at −15° C., followed by 200 mL of water. The solution is separated, the organic layer is washed 3 times with water. Combined aqueous layers are acidified to pH 6-7 with hydrochloric acid 1 N and extracted 3 times with ethyl acetate. All organic layers are combined, dried over anhydrous sodium sulfate and filtered. The filtrate is concentrated under reduced pressure and the crude product is purified by flash chromatography on a 100 g silica gel column using a dichloromethane/methanol mixture as eluent. 4.26 g of the title compound are obtained as a yellow solid.

Yield: 83%.

MH⁺: 442.6.

Step 4: 3-{5-(2-Chloropyrimidin-4-yl)-4-[3-(2,5-difluorobenzenesulfonylamino)-2-fluorophenyl]-thiazol-2-yl}-morpholine-4-carboxylic acid tert-butyl ester

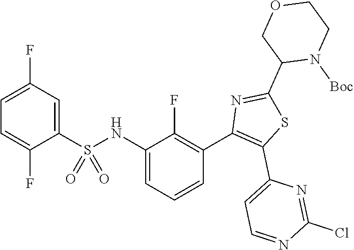 [SMILES: CC(C)(C)OC(=O)N1CCOCC1c1nc(-c2cccc(NS(=O)(=O)c3cc(F)ccc3F)c2F)c(-c2ccnc(Cl)n2)s1]

400 mg (0.91 mmol) of N-{3-[2-(2-chloropyrimidin-4-yl)-acetyl]-2-fluoro-phenyl}-2,5-difluorobenzenesulfonamide (described in the previous step) are dissolved in 4 mL of anhydrous N,N′-dimethylacetamide at room temperature under argon, then 161 mg (0.91 mmol) of N-bromosuccinimide are added and the mixture is stirred for 30 min at room temperature. 222 mg (0.91 mmol) of 3-thiocarbamoylmorpholine-4-carboxylic acid tert-butyl ester are added under argon and the reaction mixture is heated at 80° C. for 30 min. The solution is cooled to room temperature, diluted with ethyl acetate. The organic layer is washed twice with water and once with brine, dried over anhydrous sodium sulfate, filtered and evaporated under reduced pressure. Crude product is purified by flash chromatography on a 30 g silica gel column using an ethyl acetate/hexane mixture as eluent. 348 mg of the title compound are obtained.

Yield: 58%.

MH⁺: 668.8; 670.8.

Step 5: 3-{5-(2-Aminopyrimidin-4-yl)-4-[3-(2,5-difluorobenzenesulfonylamino)-2-fluorophenyl]-thiazol-2-yl}-morpholine-4-carboxylic acid tert-butyl ester

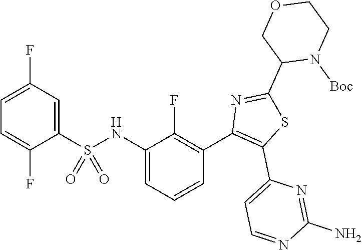 [SMILES: CC(C)(C)OC(=O)N1CCOCC1c1nc(-c2cccc(NS(=O)(=O)c3cc(F)ccc3F)c2F)c(-c2ccnc(N)n2)s1]

348 mg (0.52 mmol) of 3-{5-(2-Chloropyrimidin-4-yl)-4-[3-(2,5-difluorobenzenesulfonylamino)-2-fluorophenyl]-thiazol-2-yl}-morpholine-4-carboxylic acid tert-butyl ester (described in the previous step) are dissolved in 4 mL of a solution of ammonium hydroxide 28% and the solution is heated at 90° C. under microwave irradiation for 1 h. Mixture is diluted in 100 mL of a saturated solution of ammonium chloride and extracted 3 times with ethyl acetate. Combined organic layers are dried over anhydrous sodium sulfate, filtered and evaporated under reduced pressure. Crude product is purified by flash chromatography on a 10 g silica gel column using a dichloromethane/methanol mixture as eluent. 193 mg of the title compound are obtained as a yellow solid.

Yield: 57%.

MH⁺: 649.8.

Step 6: N-{3-[5-(2-Aminopyrimidin-4-yl)-2-morpholin-3-yl-thiazol-4-yl]-2-fluorophenyl}-2,5-difluorobenzenesulfonamide

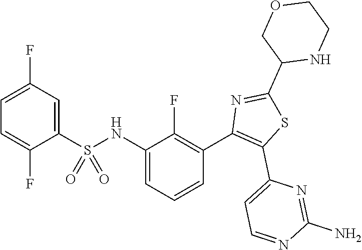 [SMILES: Nc1nccc(-c2sc(C3COCCN3)nc2-c2cccc(NS(=O)(=O)c3cc(F)ccc3F)c2F)n1]

50 mg (0.077 mmol) of 3-{5-(2-aminopyrimidin-4-yl)-4-[3-(2,5-difluorobenzenesulfonylamino)-2-fluorophenyl]-thiazol-2-yl}-morpholine-4-carboxylic acid tert-butyl ester (described in the previous step) are dissolved in 1 mL of dichloromethane, then 0.5 mL of trifluoroacetic acid are added. The solution is stirred for 1 h at room temperature. Then solvent is removed, ethyl acetate is added, and the solution is washed 3 times with a saturated solution of sodium bicarbonate. Organic layer is dried over anhydrous sodium sulfate, filtered and evaporated under reduced pressure. Crude product is purified by flash chromatography on a 10 g of silica gel column using a dichloromethane/methanol mixture as eluent, followed by another flash chromatography on a 30 g C18 column using a water/methanol mixture as eluent. 20 mg of the title compound are obtained.

Yield: 50%.

MH⁺: 549.7.

¹H NMR (DMSO-d6, 400 MHz): δ 10.63 (br s, 1H); 7.98 (d, J=5.2 Hz, 1H); 7.58-7.34 (m, 4H); 7.32-7.16 (m, 2H); 6.74 (s, 2H); 5.88 (d, J=5.1 Hz, 1H); 4.18-4.08 (m, 1H); 3.96-3.87 (m, 1H); 3.76-3.68 (m, 1H); 3.54-3.40 (m, 2H); 2.94-2.84 (m, 2H).

### Example 2: N-{3-[5-(2-Aminopyrimidin-4-yl)-2-(1-cyclopropylpiperidin-4-yl)-thiazol-4-yl]-2-fluorophenyl}-2,5-difluorobenzenesulfonamide

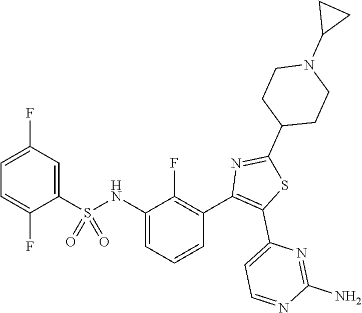 [SMILES: Nc1nccc(-c2sc(C3CCN(C4CC4)CC3)nc2-c2cccc(NS(=O)(=O)c3cc(F)ccc3F)c2F)n1]

Step 1: 1-Cyclopropylpiperidine-4-carboxamide

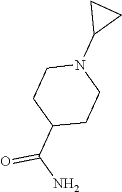 [SMILES: NC(=O)C1CCN(C2CC2)CC1]

500 mg (3.90 mmol) of piperidine-4-carboxamide are dissolved in 40 mL of methanol, then 1.18 mL (5.85 mmol) of (1-ethoxycyclopropoxy)trimethylsilane are added, followed by 0.67 mL (11.7 mmol) of acetic acid and 394 mg (6.24 mmol) of sodium cyanoborohydride. The solution is stirred at room temperature for 10 min and at 60° C. over the night. Reaction mixture is cold to room temperature, solvent is removed, and the mixture is directly purified by flash chromatography on a 30 g of silica gel column using a dichloromethane/methanol mixture as eluent. 937 mg of the title compound are obtained as a white solid.

Yield: 100%.

MH⁺: 169.2.

Step 2: 1-Cyclopropylpiperidine-4-carbothioamide

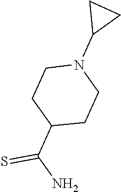 [SMILES: NC(=S)C1CCN(C2CC2)CC1]

656 mg (3.90 mmol) of 1-cyclopropylpiperidine-4-carboxamide (described in the previous step) are dissolved in 15 mL of tetrahydrofuran, then 1.36 g (3.35 mmol) of Lawesson's reagent are added and the mixture is stirred at 60° C. for 5 h. Another 0.7 g (1.73 mmol) of Lawesson's reagent is added and the mixture is stirred at 60° C. overnight. Reaction mixture is cooled at room temperature and solvent is removed under reduced pressure. Ethyl acetate is added, and the solution is washed 3 times with a saturated solution of sodium bicarbonate. Combined aqueous layers are extracted 3 times with ethyl acetate. Combinedorganic layers are dried over anhydrous sodium sulfate, filtered and evaporated under reduced pressure. Crude product is purified by flash chromatography on a 30 g of silica gel column using a dichloromethane/methanol mixture as eluent. 608 mg of the title compound are obtained as a slightly yellow solid.

Yield: 85%.

MH⁺: 185.2.

Step 3: N-{3-[5-(2-Chloropyrimidin-4-yl)-2-(1-cyclopropylpiperidin-4-yl)-thiazol-4-yl}-2-fluorophenyl]-2,5-difluorobenzenesulfonamide

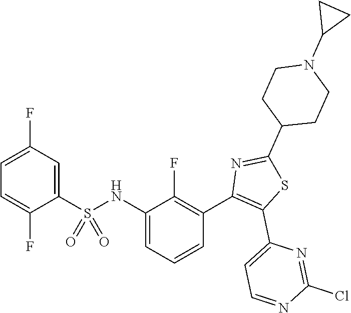 [SMILES: O=S(=O)(Nc1cccc(-c2nc(C3CCN(C4CC4)CC3)sc2-c2ccnc(Cl)n2)c1F)c1cc(F)ccc1F]

The compound is obtained by the procedure described in example 1 step 4, using 500 mg (1.13 mmol) of N-{3-[2-(2-chloropyrimidin-4-yl)-acetyl]-2-fluoro-phenyl}-2,5-difluorobenzenesulfonamide (described in example 1 step 3), 5 mL of anhydrous N,N′-dimethylacetamide, 201 mg (1.13 mmol) of N-bromosuccinimide and 208 mg (1.13 mmol) of 1-cyclopropylpiperidine-4-carbothioamide instead of 3-thiocarbamoylmorpholine-4-carboxylic acid tert-butyl ester. 142 mg of the title compound are obtained as a brown solid.

Yield: 21%.

MH⁺: 606.8; 608.8.

Step 4: N-{3-[5-(2-aminopyrimidin-4-yl)-2-(1-cyclopropylpiperidin-4-yl)-thiazol-4-yl]-2-fluorophenyl}-2,5-difluorobenzenesulfonamide

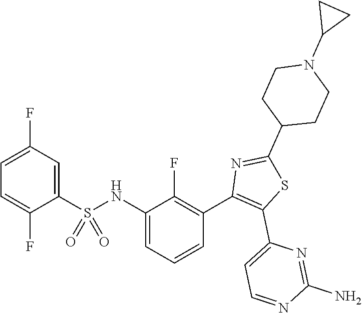 [SMILES: Nc1nccc(-c2sc(C3CCN(C4CC4)CC3)nc2-c2cccc(NS(=O)(=O)c3cc(F)ccc3F)c2F)n1]

142 mg (0.23 mmol) of N-{3-[5-(2-chloropyrimidin-4-yl)-2-(1-cyclopropylpiperidin-4-yl)-thiazol-4-yl]-2-fluorophenyl}-2,5-difluorobenzenesulfonamide (described in the previous step) are dissolved in 3 mL of a solution of ammonium hydroxide 28% and the solution is heated at 90° C. under microwave irradiation for 1 h. Mixture is diluted in 100 mL of a saturated solution of ammonium chloride and extracted 3 times with ethyl acetate. Combined organic layers are dried over anhydrous sodium sulfate, filtered and evaporated under reduced pressure. Crude product is purified by flash chromatography on a 10 g silica gel column using a dichloromethane/methanol mixture as eluent, followed by another flash chromatography on a 30 g C18 column with a water/methanol mixture as eluent. 38 mg of the title compound are obtained as a yellow solid.

Yield: 27%.

MH⁺: 587.8.

¹H NMR (DMSO-d6, 400 MHz): δ 10.65 (br s, 1H); 7.98 (d, J=5.2 Hz, 1H); 7.61-7.36 (m, 4H); 7.33-7.18 (m, 2H); 6.75 (s, 2H); 5.88 (d, J=5.1 Hz, 1H); 3.10-2.95 (m, 2H); 2.43-2.30 (m, 2H); 2.10-1.98 (m, 2H); 1.75-1.56 (m, 3H); 0.48-0.40 (m, 2H); 0.38-0.28 (m, 2H).

### Example 3: N-{3-[5-(2-Aminopyrimidin-4-yl)-2-piperidin-3-yl-thiazol-4-yl]-2-fluorophenyl}-2,5-difluorobenzenesulfonamide

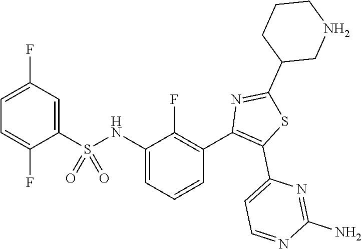 [SMILES: Nc1nccc(-c2sc(C3CCCNC3)nc2-c2cccc(NS(=O)(=O)c3cc(F)ccc3F)c2F)n1]

Step 1: 3-{5-(2-Chloropyrimidin-4-yl)-4-[3-(2,5-difluorobenzenesulfonylamino)-2-fluorophenyl]-thiazol-2-yl}-piperidine-1-carboxylic acid tert-butyl ester

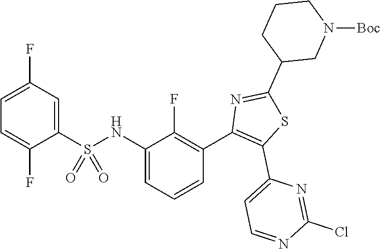 [SMILES: CC(C)(C)OC(=O)N1CCCC(c2nc(-c3cccc(NS(=O)(=O)c4cc(F)ccc4F)c3F)c(-c3ccnc(Cl)n3)s2)C1]

The compound is obtained by the procedure described in example 1 step 4, using 222 mg (0.91 mmol) of 3-thiocarbamoylpiperidine-1-carboxylic acid tert-butyl ester instead of 3-thiocarbamoylmorpholine-4-carboxylic acid tert-butyl ester. 377 mg of the title compound are obtained as a yellow solid.

Yield: 62%.

MH⁺: 666.8; 670.8.

Step 2: 3-{5-(2-Aminopyrimidin-4-yl)-4-[3-(2,5-difluorobenzenesulfonylamino)-2-fluorophenyl]-thiazol-2-yl}-piperidine-1-carboxylic acid tert-butyl ester

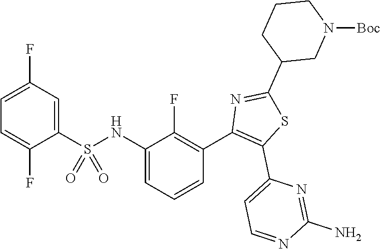 [SMILES: CC(C)(C)OC(=O)N1CCCC(c2nc(-c3cccc(NS(=O)(=O)c4cc(F)ccc4F)c3F)c(-c3ccnc(N)n3)s2)C1]

The compound is obtained by the procedure described in example 1 step 5, using 377 mg (0.57 mmol) of 3-{5-(2-chloropyrimidin-4-yl)-4-[3-(2,5-difluorobenzenesulfonylamino)-2-fluorophenyl]-thiazol-2-yl}-piperidine-1-carboxylic acid tert-butyl ester (described in the previous step). 259 mg of the title compound are obtained as a yellow solid.

Yield: 71%.

MH⁺: 647.9.

Step 3: N-{3-[5-(2-Aminopyrimidin-4-yl)-2-piperidin-3-yl-thiazol-4-yl]-2-fluorophenyl}-2,5-difluorobenzenesulfonamide

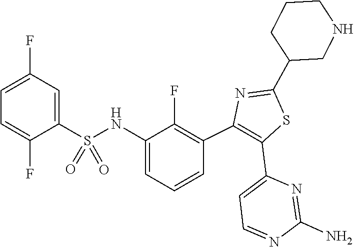 [SMILES: Nc1nccc(-c2sc(C3CCCNC3)nc2-c2cccc(NS(=O)(=O)c3cc(F)ccc3F)c2F)n1]

The compound is obtained by the procedure described in example 1 step 6, using 50 mg (0.077 mmol) of 3-{5-(2-Aminopyrimidin-4-yl)-4-[3-(2,5-difluorobenzenesulfonylamino)-2-fluorophenyl]-thiazol-2-yl}-piperidine-1-carboxylic acid tert-butyl ester (described in the previous step). 6 mg of the title compound are obtained.

Yield: 15%.

MH⁺: 547.7.

¹H NMR (DMSO-d6, 400 MHz): δ 8.65 (br s, 1H); 8.02 (d, J=5.2 Hz, 1H); 7.50-7.40 (m, 1H); 7.39-7.21 (m, 4H); 7.01-6.89 (m, 1H); 6.72 (s, 2H); 6.07 (d, J=5.0 Hz, 1H); 3.65-3.55 (m, 2H); 3.26-3.19 (m, 1H); 3.19-3.08 (m, 1H); 2.96-2.83 (m, 1H); 2.26-2.15 (m, 1H); 1.92-1.69 (m, 3H).

### Example 4: N-{3-[2-(3-Aminopropyl)-5-(2-aminopyrimidin-4-yl)-thiazol-4-yl]-2-fluorophenyl}-2,5-difluorobenzenesulfonamide trifluoroacetate

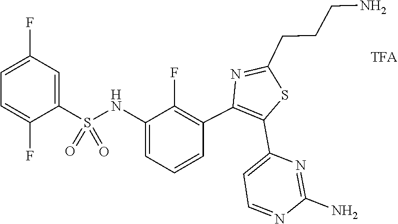 [SMILES: NCCCc1nc(-c2cccc(NS(=O)(=O)c3cc(F)ccc3F)c2F)c(-c2ccnc(N)n2)s1]

Step 1: (3-{5-(2-Chloropyrimidin-4-yl)-4-[3-(2,5-difluorobenzenesulfonylamino)-2-fluorophenyl]-thiazol-2-yl}-propyl)-carbamic acid tert-butyl ester

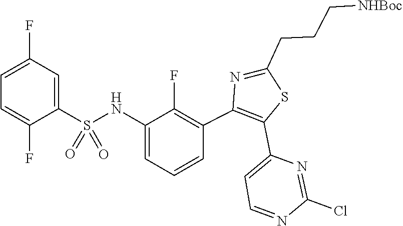 [SMILES: CC(C)(C)OC(=O)NCCCc1nc(-c2cccc(NS(=O)(=O)c3cc(F)ccc3F)c2F)c(-c2ccnc(Cl)n2)s1]

The compound is obtained by the procedure described in example 1 step 4, using 197 mg (0.90 mmol) of (3-thiocarbamoylpropyl)-carbamic acid tert-butyl ester instead of 3-thiocarbamoylmorpholine-4-carboxylic acid tert-butyl ester. 299 mg of the title compound are obtained.

Yield: 52%.

MH⁺: 640.8; 642.8.

Step 2: (3-{5-(2-Aminopyrimidin-4-yl)-4-[3-(2,5-difluorobenzenesulfonylamino)-2-fluorophenyl]-thiazol-2-yl}-propyl)-carbamic acid tert-butyl ester

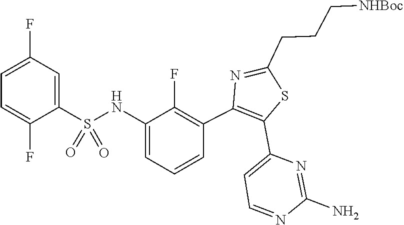 [SMILES: CC(C)(C)OC(=O)NCCCc1nc(-c2cccc(NS(=O)(=O)c3cc(F)ccc3F)c2F)c(-c2ccnc(N)n2)s1]

The compound is obtained by the procedure described in example 1 step 5, using 299 mg (1.24 mmol) of (3-{5-(2-Chloropyrimidin-4-yl)-4-[3-(2,5-difluorobenzenesulfonylamino)-2-fluorophenyl]-thiazol-2-yl}-propyl)-carbamic acid tertbutyl ester (described in the previous step). 222 mg of the title compound are obtained as an orange solid.

Yield: 76%.

MH⁺: 621.8.

Step 3: N-{3-[2-(3-Aminopropyl)-5-(2-aminopyrimidin-4-yl)-thiazol-4-yl]-2-fluorophenyl}-2,5-difluorobenzenesulfonamide trifluoroacetate

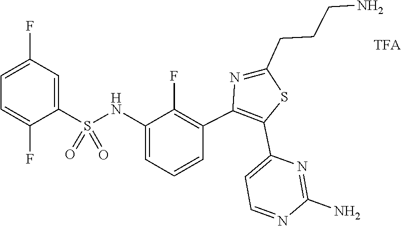 [SMILES: NCCCc1nc(-c2cccc(NS(=O)(=O)c3cc(F)ccc3F)c2F)c(-c2ccnc(N)n2)s1]

10 mg (0.016 mmol) of (3-{5-(2-aminopyrimidin-4-yl)-4-[3-(2,5-difluorobenzenesulfonylamino)-2-fluorophenyl]-thiazol-2-yl}-propyl)-carbamic acid tert-butyl ester (described in the previous step) are dissolved in 1 mL of dichloromethane, and 0.5 mL of trifluoroacetic acid is added. The solution is stirred for 1 h at room temperature, and the solvent is removed. The residue is triturated 3 times with diethyl ether and dried under vacuum over the night. 8.3 mg of the title compound are obtained.

Yield: 100%.

MH⁺: 521.7 (free base).

¹H NMR (DMSO-d6, 400 MHz): δ 10.75 (s, 1H); 8.00 (d, J=5.2 Hz, 1H); 7.80-7.63 (br s, 3H); 7.62-7.24 (m, 8H); 6.77 (s, 2H); 5.86 (d, J=5.1 Hz, 1H); 3.09 (t, J=7.4 Hz, 2H); 2.02 (m, 2H); 1.09 (t, J=7.0 Hz, 2H).

### Example 5: N-(4-{2-(1-Cyclopropylpiperidin-4-yl)-4-[3-(2,5-difluorobenzenesulfonyl-amino)-2-fluorophenyl]-thiazol-5-yl}-pyrimidin-2-yl)-acetamide

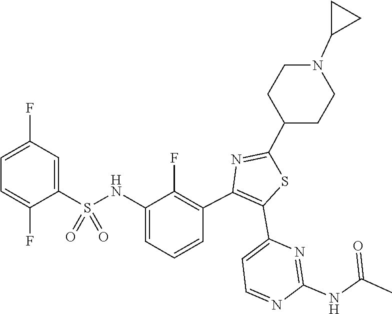 [SMILES: CC(=O)Nc1nccc(-c2sc(C3CCN(C4CC4)CC3)nc2-c2cccc(NS(=O)(=O)c3cc(F)ccc3F)c2F)n1]

30 mg (0.051 mmol) of N-{3-[5-(2-Chloropyrimidin-4-yl)-2-(1-cyclopropylpiperidin-4-yl)-thiazol-4-yl]-2-fluorophenyl}-2,5-difluorobenzenesulfonamide (described in example 2 step 4) are dissolved in 1 mL of anhydrous pyridine under argon. Then 5.8 μL (0.061 mmol) of acetic anhydride are added and the solution is stirred for 1 h at room temperature. Solvent is removed under reduced pressure, ethyl acetate is added, and the solution is washed 3 times with a solution of ammonium chloride. Organic layer is dried over anhydrous sodium sulfate, filtered and evaporated under reduced pressure. Crude product is purified by flash chromatography on a 10 g silica gel column using a dichloromethane/methanol mixture as eluent. 14 mg of the title compound are obtained as a yellow solid.

Yield: 91%.

MH⁺: 629.8.

¹H NMR (DMSO-d6, 400 MHz): δ 8.04 (d, J=5.2 Hz, 1H); 7.88-7.70 (m, 5H); 7.66-7.57 (m, 1H); 7.54 (t, J=8.1 Hz, 1H); 6.77 (s, 2H); 6.16 (d, J=5.3 Hz, 1H); 3.11-2.97 (m, 2H); 2.40-2.27 (m, 2H); 2.14-2.04 (m, 2H); 1.95 (s, 3H); 1.76-1.56 (m, 3H); 0.48-0.38 (m, 2H); 0.36-0.26 (m, 2H).

### Example 6: N-(4-{4-[3-(2,5-Difluorobenzenesulfonylamino)-2-fluorophenyl]-2-piperidin-3-yl-thiazol-5-yl}-pyrimidin-2-yl)-acetamide trifluoroacetate

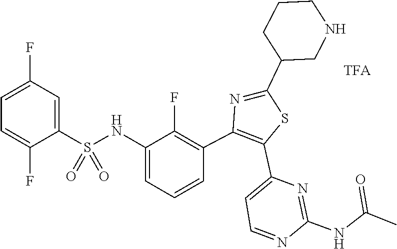 [SMILES: CC(=O)Nc1nccc(-c2sc(C3CCCNC3)nc2-c2cccc(NS(=O)(=O)c3cc(F)ccc3F)c2F)n1]

Step 1: 3-{5-(2-Acetylaminopyrimidin-4-yl)-4-[3-(2,5-difluorobenzenesulfonylamino)-2-fluorophenyl]-thiazol-2-yl}-piperidine-1-carboxylic acid tert-butyl ester

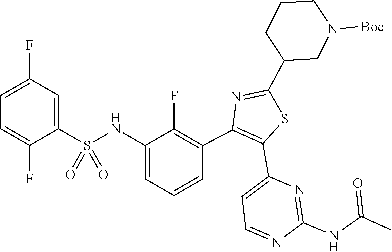 [SMILES: CC(=O)Nc1nccc(-c2sc(C3CCCN(C(=O)OC(C)(C)C)C3)nc2-c2cccc(NS(=O)(=O)c3cc(F)ccc3F)c2F)n1]

30 mg (0.046 mmol) of 3-{5-(2-aminopyrimidin-4-yl)-4-[3-(2,5-difluorobenzenesulfonylamino)-2-fluorophenyl]-thiazol-2-yl}-piperidine-1-carboxylic acid tert-butyl ester (described in example 3 step 2) are dissolved in 1 mL of anhydrous pyridine under argon. Then 5.3 μL (0.055 mmol) of acetic anhydride are added and the solution is stirred for 1 h at room temperature. Solvent is removed under reduced pressure, ethyl acetate is added, and the solution is washed 3 times with a solution of ammonium chloride. Organic layer is dried over anhydrous sodium sulfate, filtered and evaporated under reduced pressure. 36 mg of crude product are obtained and directly used in the next step without further purification.

Yield: 100%.

MH⁺: 689.7.

Step 2: N-(4-{4-[3-(2,5-Difluorobenzenesulfonylamino)-2-fluorophenyl]-2-piperidin-3-yl-thiazol-5-yl}-pyrimidin-2-yl)-acetamide trifluoroacetate

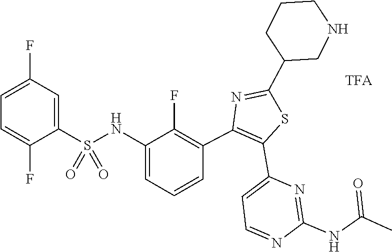 [SMILES: CC(=O)Nc1nccc(-c2sc(C3CCCNC3)nc2-c2cccc(NS(=O)(=O)c3cc(F)ccc3F)c2F)n1]

21 mg (0.030 mmol) of 3-{5-(2-acetylaminopyrimidin-4-yl)-4-[3-(2,5-difluorobenzenesulfonylamino)-2-fluorophenyl]-thiazol-2-yl}-piperidine-1-carboxylic acid tert-butyl ester (described in the previous step) are dissolved in 1 mL of dichloromethane, and 0.5 mL of trifluoroacetic acid is added. The solution is stirred for 1 h at room temperature. Solvent is removed under reduced pressure. Residue is triturated 3 times with diethyl ether and dried under vacuum over the night. 14.5 mg of the title compound are obtained.

Yield: 80%.

MH⁺: 589.7 (free base).

¹H NMR (DMSO-d6, 400 MHz): δ 8.85-8.70 (m, 1H); 8.63-8.47 (m, 1H); 8.07 (d, J=5.2 Hz, 1H); 7.88-7.70 (m, 5H); 7.67-7.52 (m, 2H); 6.81 (s, 2H); 6.18 (d, J=5.0 Hz, 1H); 3.73-3.62 (m, 2H); 3.03-2.87 (m, 1H); 2.30-2.20 (m, 1H); 1.95 (s, 3H); 1.99-1.88 (m, 1H); 1.88-1.69 (m, 3H).

### Example 7: N-(4-{4-[3-(2,5-Difluorobenzenesulfonylamino)-2-fluorophenyl]-2-morpholin-3-yl-thiazol-5-yl}-pyrimidin-2-yl)-acetamide

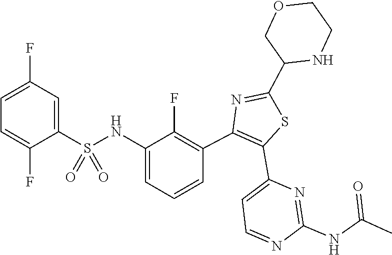 [SMILES: CC(=O)Nc1nccc(-c2sc(C3COCCN3)nc2-c2cccc(NS(=O)(=O)c3cc(F)ccc3F)c2F)n1]

Step 1: 3-{5-(2-Acetylaminopyrimidin-4-yl)-4-[3-(2,5-difluorobenzenesulfonylamino)-2-fluorophenyl]-thiazol-2-yl}-morpholine-4-carboxylic acid tert-butyl ester

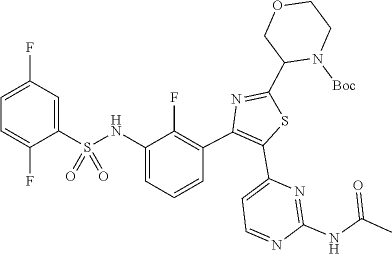 [SMILES: CC(=O)Nc1nccc(-c2sc(C3COCCN3C(=O)OC(C)(C)C)nc2-c2cccc(NS(=O)(=O)c3cc(F)ccc3F)c2F)n1]

The compound is obtained by the procedure described in example 6 step 1, using 30 mg (0.046 mmol) of 3-{5-(2-aminopyrimidin-4-yl)-4-[3-(2,5-difluorobenzenesulfonylamino)-2-fluorophenyl]-thiazol-2-yl}-morpholine-4-carboxylic acid tert-butyl ester (described in example 1 step 5) and 5.3 μL (0.055 mmol) of acetic anhydride. 39 mg of crude product are obtained and directly used in the next step without further purification.

Yield: 100%.

MH⁺: 691.7.

Step 2: N-(4-{4-[3-(2,5-Difluorobenzenesulfonylamino)-2-fluorophenyl]-2-morpholin-3-yl-thiazol-5-yl}-pyrimidin-2-yl)-acetamide

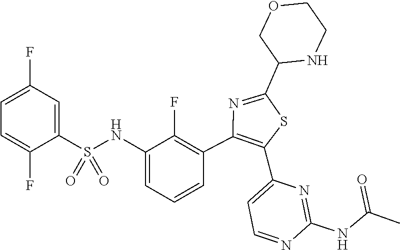 [SMILES: CC(=O)Nc1nccc(-c2sc(C3COCCN3)nc2-c2cccc(NS(=O)(=O)c3cc(F)ccc3F)c2F)n1]

32 mg (0.046 mmol) of 3-{5-(2-acetylaminopyrimidin-4-yl)-4-[3-(2,5-difluorobenzenesulfonylamino)-2-fluorophenyl]-thiazol-2-yl}-morpholine-4-carboxylic acid tert-butyl ester (described in the previous step) are dissolved in 1 mL of dichloromethane, and 0.5 mL of trifluoroacetic acid is added. The solution is stirred for 1 h at room temperature. Solvent is removed under reduced pressure and the residue is directly purified on a 10 g silica gel column with a dichloromethane/methanol mixture as eluent. 8.0 mg of the title compound are obtained.

Yield: 30%.

MH⁺: 591.7.

¹H NMR (DMSO-d6, 400 MHz): δ 8.05 (d, J=5.2 Hz, 1H); 7.85-7.71 (m, 4H); 7.66-7.57 (m, 1H); 7.54 (t, J=8.0 Hz, 1H); 6.76 (s, 2H); 6.17 (d, J=5.0 Hz, 1H); 4.21-4.14 (m, 1H); 4.00-3.92 (m, 1H); 3.77-3.69 (m, 1H); 3.57-3.44 (m, 3H); 2.96-2.83 (m, 2H); 1.94 (s, 3H).

### Example 8: N-(4-{2-(3-Aminopropyl)-4-[3-(2,5-difluorobenzenesulfonylamino)-2-fluorophenyl]-thiazol-5-yl}-pyrimidin-2-yl)-acetamide

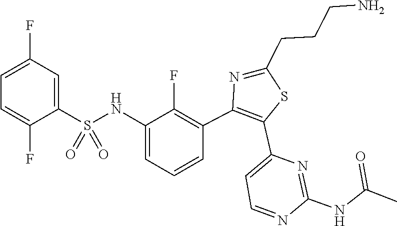 [SMILES: CC(=O)Nc1nccc(-c2sc(CCCN)nc2-c2cccc(NS(=O)(=O)c3cc(F)ccc3F)c2F)n1]

Step 1: (3-{5-(2-Acetylaminopyrimidin-4-yl)-4-[3-(2,5-difluorobenzenesulfonylamino)-2-fluorophenyl]-thiazol-2-yl}-propyl)-carbamic acid tert-butyl ester

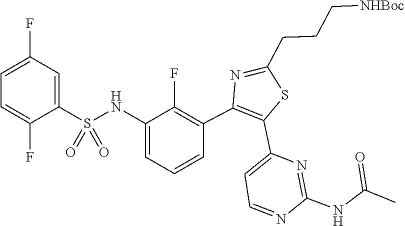 [SMILES: CC(=O)Nc1nccc(-c2sc(CCCNC(=O)OC(C)(C)C)nc2-c2cccc(NS(=O)(=O)c3cc(F)ccc3F)c2F)n1]

The compound is obtained by the procedure described in example 6 step 1, using 30 mg (0.048 mmol) of (3-{5-(2-aminopyrimidin-4-yl)-4-[3-(2,5-difluorobenzenesulfonylamino)-2-fluorophenyl]-thiazol-2-yl}-propyl)-carbamic acid tert-butyl ester (described in example 4 step 2) and 5.5 μL (0.058 mmol) of acetic anhydride. 32 mg of crude product are obtained and directly used in the next step without further purification.

Yield: 100%.

MH⁺: 663.7.

Step 2: N-(4-{2-(3-Aminopropyl)-4-[3-(2,5-difluorobenzenesulfonylamino)-2-fluorophenyl]-thiazol-5-yl}-pyrimidin-2-yl)-acetamide

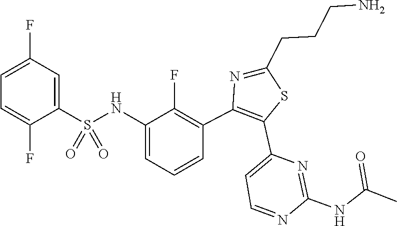 [SMILES: CC(=O)Nc1nccc(-c2sc(CCCN)nc2-c2cccc(NS(=O)(=O)c3cc(F)ccc3F)c2F)n1]

32 mg (0.048 mmol) of (3-{5-(2-acetylaminopyrimidin-4-yl)-4-[3-(2,5-difluorobenzenesulfonylamino)-2-fluorophenyl]-thiazol-2-yl}-propyl)-carbamic acid tert-butyl ester (described in the previous step) are dissolved in 1 mL of dichloromethane, and 0.5 mL of trifluoroacetic acid is added. The solution is stirred for 1 h at room temperature. Solvent is removed under reduced pressure and the residue is directly purified on a 10 g silica gel column using a dichloromethane/methanol/ammonium hydroxide 0.5% mixture as eluent. 11.0 mg of the title compound are obtained.

Yield: 40%.

MH⁺: 563.7.

¹H NMR (DMSO-d6, 400 MHz): δ 10.75 (s, 1H); 7.98 (d, J=5.2 Hz, 1H); 7.957.85 (br s, 1H); 7.64-7.21 (m, 5H); 6.75 (s, 2H); 5.85 (d, J=5.2 Hz, 1H); 3.16-3.08 (m, 2H); 2.99 (t, J=7.6 Hz, 2H); 1.91-1.79 (m, 2H); 1.79 (s, 3H).

### Example 9: N-(4-{4-[3-(2,5-Difluorobenzenesulfonylamino)-2-fluoro-phenyl]-2-morpholin-3-yl-thiazol-5-yl}-pyrimidin-2-yl)-acrylamide trifluoroacetate

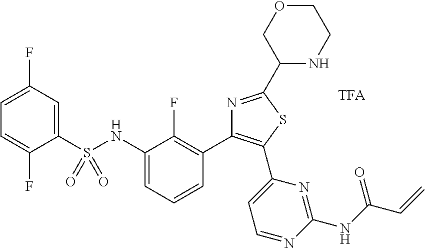 [SMILES: C=CC(=O)Nc1nccc(-c2sc(C3COCCN3)nc2-c2cccc(NS(=O)(=O)c3cc(F)ccc3F)c2F)n1]

Step 1: 3-{5-(2-Acryloylaminopyrimidin-4-yl)-4-[3-(2,5-difluorobenzenesulfonylamino)-2-fluorophenyl]-thiazol-2-yl}-morpholine-4-carboxylic acid tert-butyl ester

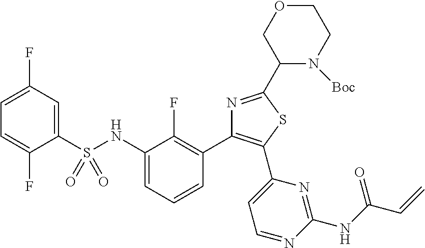 [SMILES: C=CC(=O)Nc1nccc(-c2sc(C3COCCN3C(=O)OC(C)(C)C)nc2-c2cccc(NS(=O)(=O)c3cc(F)ccc3F)c2F)n1]

15 mg (0.023 mmol) of 3-{5-(2-aminopyrimidin-4-yl)-4-[3-(2,5-difluorobenzenesulfonylamino)-2-fluorophenyl]-thiazol-2-yl}-morpholine-4-carboxylic acid tert-butyl ester (described in example 1 step 5) are dissolved in 1 mL of anhydrous dichloromethane under argon, then 3.8 μL (0.028 mmol) of triethylamine and 2.2 μL (0.023 mmol) of acryloyl chloride are added. The solution is stirred for 1 h at room temperature. Solvent is removed under reduced pressure, ethyl acetate is added, and the solution is washed 3 times with water and 1 time with brine. Organic layer is dried over anhydrous sodium sulfate, filtered and evaporated under reduced pressure. Crude product is purified by flash chromatography on a 30 g C18 column using a water/methanol mixture as eluent. 7 mg of the title compound are obtained.

Yield: 44%.

MH⁺: 703.8.

Step 2: N-(4-{4-[3-(2,5-Difluorobenzenesulfonylamino)-2-fluoro-phenyl]-2-morpholin-3-yl-thiazol-5-yl}-pyrimidin-2-yl)-acrylamide trifluoroacetate

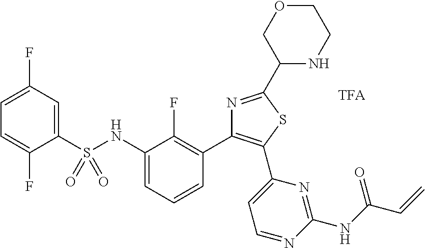 [SMILES: C=CC(=O)Nc1nccc(-c2sc(C3COCCN3)nc2-c2cccc(NS(=O)(=O)c3cc(F)ccc3F)c2F)n1]

7 mg (0.010 mmol) of 3-{5-(2-acryloylaminopyrimidin-4-yl)-4-[3-(2,5-difluorobenzenesulfonylamino)-2-fluorophenyl]-thiazol-2-yl}-morpholine-4-carboxylic acid tert-butyl ester (described in the previous step) are dissolved in 3 mL of dichloromethane, and 1 mL of trifluoroacetic acid is added. The solution is stirred for 1 h at room temperature. Solvent is removed under reduced pressure. Residue is triturated 3 times with diethyl ether and dried under vacuum for the night. 2.2 mg of the title compound are obtained as a pale yellow solid.

Yield: 36%.

MH⁺: 603.7.

### Example 10: N-(4-{2-(3-Aminopropyl)-4-[3-(2,5-difluorobenzenesulfonylamino)-2-fluorophenyl]-thiazol-5-yl}-pyrimidin-2-yl)-acrylamide trifluoroacetate

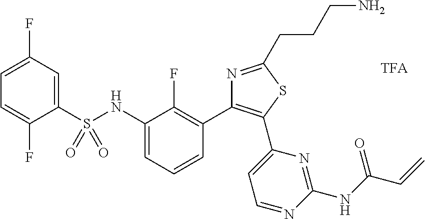 [SMILES: C=CC(=O)Nc1nccc(-c2sc(CCCN)nc2-c2cccc(NS(=O)(=O)c3cc(F)ccc3F)c2F)n1]

Step 1: (3-{5-(2-Acryloylaminopyrimidin-4-yl)-4-[3-(2,5-difluorobenzenesulfonylamino)-2-fluorophenyl]-thiazol-2-yl}-propyl)-carbamic acid tert-butyl ester

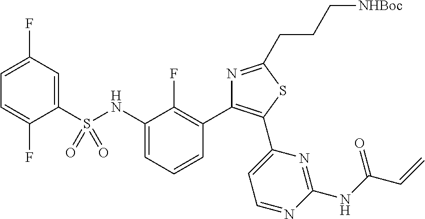 [SMILES: C=CC(=O)Nc1nccc(-c2sc(CCCNC(=O)OC(C)(C)C)nc2-c2cccc(NS(=O)(=O)c3cc(F)ccc3F)c2F)n1]

27 mg (0.043 mmol) of (3-{5-(2-aminopyrimidin-4-yl)-4-[3-(2,5-difluorobenzenesulfonylamino)-2-fluorophenyl]-thiazol-2-yl}-propyl)-carbamic acid tert-butyl ester (described in example 4 step 2) are dissolved in 1 mL of anhydrous dichloromethane under argon, then 6.7 μL (0.048 mmol) of triethylamine and 3.9 μL (0.048 mmol) of acryloyl chloride are added at 0° C. The solution is stirred at 0° C. for 10 min and at room temperature for 1 h. Solvent is removed under reduced pressure, ethyl acetate is added, and the solution is washed 3 times with water and 1 time with brine. Organic layer is dried over anhydrous sodium sulfate, filtered and evaporated under reduced pressure. Crude product is purified by flash chromatography on a 30 g C18 column using a water/methanol mixture as eluent. 19 mg of the title compound are obtained.

Yield: 66%.

MH⁺: 675.9.

Step 2: N-(4-{2-(3-Aminopropyl)-4-[3-(2,5-difluoro-benzenesulfonylamino)-2-fluorophenyl]-thiazol-5-yl}-pyrimidin-2-yl)-acrylamide trifluoroacetate

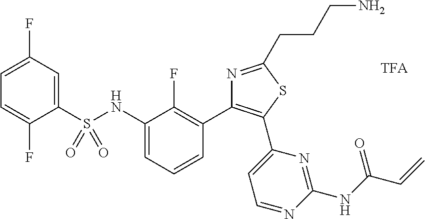 [SMILES: C=CC(=O)Nc1nccc(-c2sc(CCCN)nc2-c2cccc(NS(=O)(=O)c3cc(F)ccc3F)c2F)n1]

12 mg (0.018 mmol) of (3-{5-(2-acryloylaminopyrimidin-4-yl)-4-[3-(2,5-difluorobenzenesulfonylamino)-2-fluorophenyl]-thiazol-2-yl}-propyl)-carbamic acid tert-butyl ester (described in the previous step) are dissolved in 3 mL of dichloromethane, and 1 mL of trifluoroacetic acid is added. The solution is stirred for 1 h at room temperature. Solvent is removed under reduced pressure. Residue is triturated 3 times with diethyl ether and dried under vacuum for the night. 8.3 mg of the title compound are obtained as a pale yellow solid.

Yield: 83%.

MH⁺: 575.8.

### Example 11: 3-{5-(2-Acryloylaminopyrimidin-4-yl)-4-[3-(2,5-difluoro-benzenesulfonylamino)-2-fluorophenyl]-thiazol-2-yl}-piperidine-1-carboxylic acid tert-butyl ester

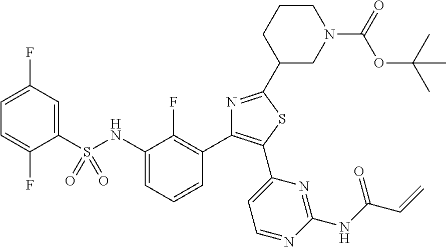 [SMILES: C=CC(=O)Nc1nccc(-c2sc(C3CCCN(C(=O)OC(C)(C)C)C3)nc2-c2cccc(NS(=O)(=O)c3cc(F)ccc3F)c2F)n1]

30 mg (0.046 mmol) of 3-{5-(2-aminopyrimidin-4-yl)-4-[3-(2,5-difluorobenzenesulfonylamino)-2-fluorophenyl]-thiazol-2-yl}-piperidine-1-carboxylic acid tert-butyl ester (described in example 3 step 2) are dissolved in 1 mL of anhydrous dichloromethane under argon, then 7.7 μL (0.055 mmol) of triethylamine and 7.5 μL (0.093 mmol) of acryloyl chloride are added. The solution is stirred at room temperature for 1 h. Solvent is removed under reduced pressure, ethyl acetate is added, and the solution is washed 3 times with water and 1 time with brine. Organic layer is dried over anhydrous sodium sulfate, filtered and evaporated under reduced pressure. Crude product is purified by flash chromatography on a 30 g C18 column using a water/methanol mixture as eluent. 5 mg of the title compound are obtained.

Yield: 13%.

MH⁺: 701.8.

### Example 12: Butane-2-sulfonic acid {3-[5-(2-aminopyrimidin-4-yl)-2-piperidin-4-yl-thiazol-4-yl]-2-fluorophenyl}-amide

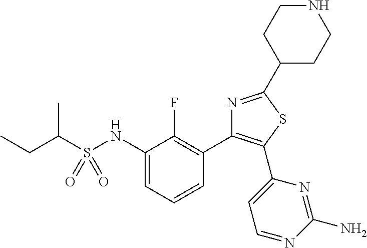 [SMILES: CCC(C)S(=O)(=O)Nc1cccc(-c2nc(C3CCNCC3)sc2-c2ccnc(N)n2)c1F]

Step 1: 3-(Butane-2-sulfonylamino)-2-fluorobenzoic acid methyl ester

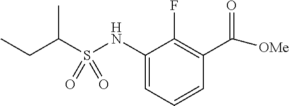 [SMILES: CCC(C)S(=O)(=O)Nc1cccc(C(=O)OC)c1F]

To a solution of 1.49 g (8.81 mmol) of 3-amino-2-fluorobenzoic acid methyl ester (described in example 1 step 1) in 15 mL of anhydrous pyridine, are added under argon 1.65 g mL (10.6 mmol) of butane-2-sulfonyl chloride at 0° C. The mixture is stirred at 0° C. for 20 min, then at room temperature for the night. The mixture is concentrated under reduced pressure, dissolved in ethyl acetate, washed 3 times with a saturated solution of sodium bicarbonate and once with brine. Organic layer is then dried over anhydrous sodium sulfate and filtered. The filtrate is concentrated under reduced pressure and the crude product is purified by flash chromatography on a 30 g silica gel column using a dichloromethane/methanol mixture as eluent, followed by another flash chromatography on a 120 g C18 column using a water/acetonitrile mixture as eluent. 750 mg of the title compound are obtained.

Yield: 30%.

MH⁺: 290.3.

Step 2: Butane-2-sulfonic acid {3-[2-(2-chloropyrimidin-4-yl)-acetyl]-2-fluorophenyl}-amide

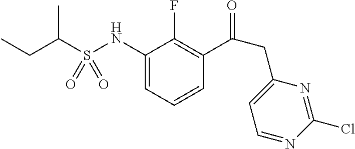 [SMILES: CCC(C)S(=O)(=O)Nc1cccc(C(=O)Cc2ccnc(Cl)n2)c1F]

To a solution of 750 mg (2.59 mmol) of 3-(butane-2-sulfonylamino)-2-fluorobenzoic acid methyl ester (described in the previous step) in 7 mL of anhydrous tetrahydrofuran, are slowly added, at −15° C. under argon, 9 mL (9 mmol) of a solution of lithium bis(trimethylsilyl)amide (1 M in tetrahydrofuran). The mixture is stirred at −15° C. for 20 min then, a solution of 400 mg (3.11 mmol) of 2-chloro-4-methylpyrimidine in 2 mL of anhydrous tetrahydrofuran is slowly added while keeping the bath temperature between −20 and −15° C. The reaction is stirred at −15° C. for 30 min then, 5 mL of a saturated solution of ammonium chloride is slowly added at −15° C., followed by 100 mL of ethyl acetate and 100 mL of water. The solution is separated, the organic layer is washed twice with water. Combined aqueous layers are acidified to pH 6-7 with hydrochloric acid 1 N and extracted 3 times with ethyl acetate. Combined organic layers are dried over anhydrous sodium sulfate and filtered. The filtrate is concentrated under reduced pressure and crude product is purified by flash chromatography on a 30 g silica gel column using a dichloromethane/methanol mixture as eluent. 882 mg of the title compound are obtained as a dark orange oil.

Yield: 88%.

MH⁺: 386.3; 388.3.

Step 3: 4-[4-[3-(Butane-2-sulfonylamino)-2-fluorophenyl]-5-(2-chloropyrimidin4-yl)-thiazol-2-yl]-piperidine-1-carboxylic acid tert-butyl ester

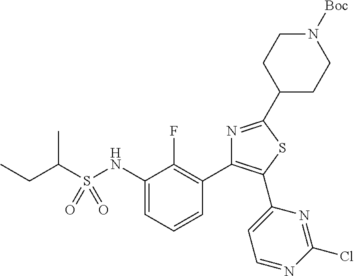 [SMILES: CCC(C)S(=O)(=O)Nc1cccc(-c2nc(C3CCN(C(=O)OC(C)(C)C)CC3)sc2-c2ccnc(Cl)n2)c1F]

100 mg (0.26 mmol) of butane-2-sulfonic acid {3-[2-(2-chloropyrimidin-4-yl)-acetyl]-2-fluorophenyl}-amide (described in the previous step) are dissolved in 2 mL of anhydrous N,N′-dimethylacetamide at room temperature under argon, then 46 mg (0.26 mmol) of N-bromosuccinimide are added and the mixture is stirred for 30 min at room temperature. 63 mg (0.26 mmol) of 4-thiocarbamoylpiperidine-1-carboxylic acid tert-butyl ester are added under argon and the reaction mixture is heated at 80° C. for 30 min. The solution is cooled to room temperature, diluted with ethyl acetate. The solution is washed 3 times with water and once with brine, dried over anhydrous sodium sulfate, filtered and evaporated under reduced pressure. Crude product is purified by flash chromatography on a 10 g silica gel column using an ethyl acetate/hexane mixture as eluent. 80 mg of the title compound are obtained.

Yield: 50%.

MH⁺: 610.5; 612.5.

Step 4: 4-{5-(2-Aminopyrimidin-4-yl)-4-[3-(butane-2-sulfonylamino)-2-fluorophenyl]-thiazol-2-yl}-piperidine-1-carboxylic acid tert-butyl ester

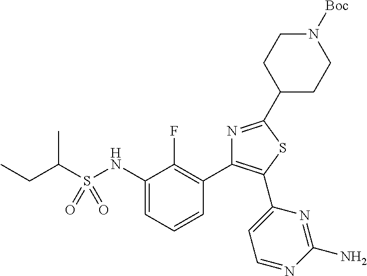 [SMILES: CCC(C)S(=O)(=O)Nc1cccc(-c2nc(C3CCN(C(=O)OC(C)(C)C)CC3)sc2-c2ccnc(N)n2)c1F]

80 mg (0.13 mmol) of 4-[4-[3-(butane-2-sulfonylamino)-2-fluorophenyl]-5-(2-chloropyrimidin-4-yl)-thiazol-2-yl]-piperidine-1-carboxylic acid tert-butyl ester (described in the previous step) are dissolved in 3 mL of a solution of ammonium hydroxide 28% and the solution is heated at 90° C. under microwave irradiation for 1 h. Mixture is diluted in 50 mL of a saturated solution of ammonium chloride and extracted 3 times with ethyl acetate. Combined organic layers are dried over anhydrous sodium sulfate, filtered and evaporated under reduced pressure. Crude product is purified by flash chromatography on a 10 g silica gel column using a dichloromethane/methanol mixture as eluent. 63 mg of the title compound are obtained as a yellow solid.

Yield: 81%.

MH⁺: 591.4.

Step 5: Butane-2-sulfonic acid {3-[5-(2-aminopyrimidin-4-yl)-2-piperidin-4-yl-thiazol-4-yl]-2-fluorophenyl}-amide

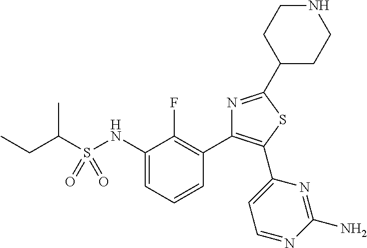 [SMILES: CCC(C)S(=O)(=O)Nc1cccc(-c2nc(C3CCNCC3)sc2-c2ccnc(N)n2)c1F]

63 mg (0.11 mmol) of 4-{5-(2-aminopyrimidin-4-yl)-4-[3-(butane-2-sulfonylamino)-2-fluorophenyl]-thiazol-2-yl}-piperidine-1-carboxylic acid tert-butyl ester (described in the previous step) are dissolved in 1 mL of dichloromethane and 0.5 mL of trifluoroacetic acid are added. The solution is stirred for 1 h at room temperature. Then solvent is removed, ethyl acetate is added, and the solution is washed 3 times with a saturated solution of sodium bicarbonate. Organic layer is dried over anhydrous sodium sulfate, filtered and evaporated under reduced pressure. Crude product is purified by flash chromatography on a 10 g of silica gel column using a dichloromethane/methanol mixture as eluent, followed by another flash chromatography on a 30 g C18 column using a water/methanol mixture as eluent. 13 mg of the title compound are obtained.

Yield: 26%.

MH⁺: 491.4.

¹H NMR (DMSO-d6, 400 MHz): δ 8.05 (d, J=5.2 Hz, 1H); 7.59-7.49 (m, 1H); 7.30-7.20 (m, 2H); 6.75 (s, 2H); 6.09 (d, J=5.2 Hz, 1H); 3.20-3.12 (m, 1H); 3.12-3.02 (m, 2H); 3.74-3.63 (m, 2H); 2.10-1.99 (m, 2H); 1.97-1.84 (m, 1H); 1.73-1.59 (m, 2H); 1.49-1.32 (m, 1H); 1.20 (d, J=6.8 Hz, 3H); 0.88 (t, J=7.4 Hz, 3H).

### Example 13: N-{3-[5-(2-Aminopyrimidin-4-yl)-2-morpholin-3-yl-thiazol-4-yl]-5-chloro-2-fluorophenyl}-2,5-difluorobenzenesulfonamide

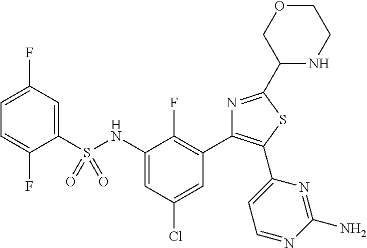 [SMILES: Nc1nccc(-c2sc(C3COCCN3)nc2-c2cc(Cl)cc(NS(=O)(=O)c3cc(F)ccc3F)c2F)n1]

Step 1: 3-Amino-5-chloro-2-fluorobenzoic acid methyl ester

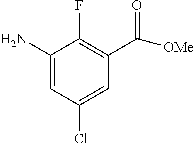 [SMILES: COC(=O)c1cc(Cl)cc(N)c1F]

500 mg (2.64 mmol) of 3-amino-5-chloro-2-fluorobenzoic acid are dissolved in 5 mL of anhydrous methanol under argon. 202 μL (2.77 mmol) of thionyl chloride are slowly added at 0° C., and the reaction is then refluxed for 3 h. The solution is cooled to room temperature. Solvent is removed under reduced pressure. Reaction mixture is quenched thanks to a saturated solution of sodium bicarbonate, extracted 3 times with ethyl acetate. Combined organic layers are dried over anhydrous sodium sulfate and filtered. The filtrate is concentrated under reduced pressure and crude product is directly used in the next step without further purification. 432 mg of the title compound are obtained as a yellow solid.

Yield: 80%.

Step 2: 5-Chloro-3-(2,5-difluorobenzenesulfonylamino)-2-fluorobenzoic acid methyl ester

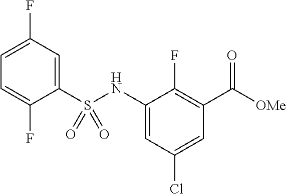 [SMILES: COC(=O)c1cc(Cl)cc(NS(=O)(=O)c2cc(F)ccc2F)c1F]

To a solution of 432 mg (2.12 mmol) of 3-amino-5-chloro-2-fluorobenzoic acid methyl ester (described in the previous step) in 5 mL of anhydrous pyridine, are added under argon, 342 μL (2.55 mmol) of 2,5-difluorobenzenesulfonyl chloride at 0° C. The mixture is stirred at 0° C. for 20 min, then at room temperature over the night. The mixture is concentrated under reduced pressure, dissolved in dichloromethane, washed 4 times with hydrochloric acid 0.5N and once with brine, dried over anhydrous sodium sulfate and filtered. The filtrate is concentrated under reduced pressure and crude product is purified by flash chromatography on a 30 g silica gel column using a dichloromethane/methanol mixture as eluent. 438 mg of the title compound are obtained as a yellow solid.

Yield: 55%.

MH⁺: 380.4; 382.5.

Step 3: N-{5-Chloro-3-[2-(2-chloropyrimidin-4-yl)-acetyl]-2-fluorophenyl}-2,5-difluorobenzenesulfonamide

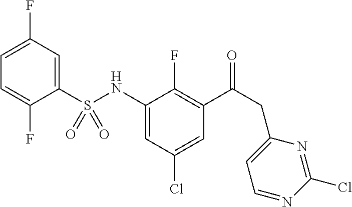 [SMILES: O=C(Cc1ccnc(Cl)n1)c1cc(Cl)cc(NS(=O)(=O)c2cc(F)ccc2F)c1F]

To a solution of 438 mg (1.16 mmol) of 5-chloro-3-(2,5-difluorobenzenesulfonylamino)-2-fluorobenzoic acid methyl ester (described in the previous step) in 4 mL of anhydrous tetrahydrofuran, are slowly added at −15° C. under argon 4 mL (4 mmol) of a solution of lithium bis(trimethylsilyl)amide (1 M in tetrahydrofuran). The reaction is stirred at −15° C. for 20 min, then a solution of 178 mg (1.38 mmol) of 2-chloro-4methylpyrimidine in 1 mL of anhydrous tetrahydrofuran is slowly added while keeping the bath temperature between −20° C. and −15° C. The reaction is stirred at this temperature for another 30 min, and 1.5 mL of a saturated solution of ammonium chloride is slowly added at 15° C., followed by 10 mL of ethyl acetate and 10 mL of water. The solution is separated, the organic layer is washed 3 times with water. Combined aqueous layers are acidified to pH 6-7 with hydrochloric acid 1 N and extracted 3 times with ethyl acetate. Combined organic layers are dried over anhydrous sodium sulfate and filtered. The filtrate is concentrated under reduced pressure and crude product is purified by flash chromatography on a 30 g silica gel column using a dichloromethane/methanol mixture as eluent. 415 mg of the title compound are obtained as an orange solid.

Yield: 76%.

MH⁺: 476.4; 478.4; 480.5.

Step 4: 3-[4-[5-Chloro-3-(2,5-difluorobenzenesulfonylamino)-2-fluorophenyl]-5-(2-chloropyrimidin-4-yl)-thiazol-2-yl]-morpholine-4-carboxylic acid tert-butyl ester

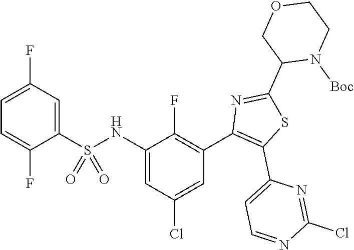 [SMILES: CC(C)(C)OC(=O)N1CCOCC1c1nc(-c2cc(Cl)cc(NS(=O)(=O)c3cc(F)ccc3F)c2F)c(-c2ccnc(Cl)n2)s1]

200 mg (0.42 mmol) of N-{5-chloro-3-[2-(2-chloropyrimidin-4-yl)-acetyl]-2-fluorophenyl}-2,5-difluorobenzenesulfonamide (described in the previous step) are dissolved in 2 mL of anhydrous N,N′-dimethylacetamide at room temperature under argon, then 75 mg (0.42 mmol) of N-bromosuccinimide are added and the mixture is stirred for 30 min at room temperature. 103 mg (0.42 mmol) of 3-thiocarbamoylmorpholine-4-carboxylic acid tert-butyl ester are added under argon and the reaction mixture is heated at 80° C. for 30 min. The solution is cooled to room temperature, diluted with ethyl acetate, then washed twice with water and once with brine, dried over anhydrous sodium sulfate, filtered and evaporated under reduced pressure. Crude product is purified by flash chromatography on a 10 g silica gel column using an ethyl acetate/hexane mixture as eluent. 140 mg of the title compound are obtained.

Yield: 47%.

MH⁺: 702.8; 704.8; 706.8.

Step 5: 3-{5-(2-Aminopyrimidin-4-yl)-4-[5-chloro-3-(2,5-difluorobenzenesulfonylamino)-2-fluorophenyl]-thiazol-2-yl}-morpholine-4-carboxylic acid tert-butyl ester

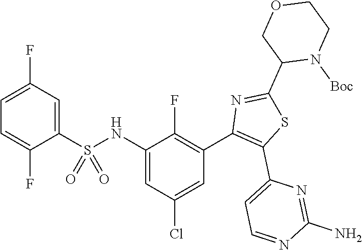 [SMILES: CC(C)(C)OC(=O)N1CCOCC1c1nc(-c2cc(Cl)cc(NS(=O)(=O)c3cc(F)ccc3F)c2F)c(-c2ccnc(N)n2)s1]

140 mg (0.20 mmol) of 3-[4-[5-chloro-3-(2,5-difluorobenzenesulfonylamino)-2-fluorophenyl]-5-(2-chloropyrimidin-4-yl)-thiazol-2-yl]-morpholine-4-carboxylic acid tert-butyl ester (described in the previous step) are dissolved in 2 mL of a solution of ammonium hydroxide 28% and the solution is heated at 90° C. under microwave irradiation for 1 h. Mixture is diluted in 50 mL of a saturated solution of ammonium chloride and extracted 3 times with ethyl acetate. Combined organic layers are dried over anhydrous sodium sulfate, filtered and evaporated under reduced pressure. Crude product is directly used in the next step without further purification (92 mg of a brown solid).

Yield: 68%.

MH⁺: 683.8; 685.8.

Step 6: N-{3-[5-(2-Aminopyrimidin-4-yl)-2-morpholin-3-yl-thiazol-4-yl]-5-chloro-2-fluorophenyl}-2,5-difluorobenzenesulfonamide

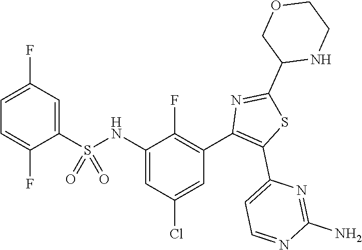 [SMILES: Nc1nccc(-c2sc(C3COCCN3)nc2-c2cc(Cl)cc(NS(=O)(=O)c3cc(F)ccc3F)c2F)n1]

92 mg (0.13 mmol) of 3-{5-(2-aminopyrimidin-4-yl)-4-[5-chloro-3-(2,5-difluorobenzenesulfonylamino)-2-fluorophenyl]-thiazol-2-yl}-morpholine-4-carboxylic acid tert-butyl ester (described in the previous step) are dissolved in 1 mL of dichloromethane and 0.5 mL of trifluoroacetic acid are added. The solution is stirred for 1 h at room temperature. Then solvent is removed, ethyl acetate is added, and the solution is washed 3 times with a saturated solution of sodium bicarbonate. Organic layer is dried over anhydrous sodium sulfate, filtered and evaporated under reduced pressure. Crude product is purified by flash chromatography on a 10 g of silica gel column using a dichloromethane/methanol mixture as eluent, followed by another flash chromatography on a 30 g C18 column using a water/methanol mixture as eluent. 33 mg of the title compound are obtained as a slightly orange solid.

Yield: 44%.

MH⁺: 583.7; 585.7.

¹H NMR (DMSO-d6, 400 MHz): δ 8.07 (d, J=5.2 Hz, 1H); 7.55-7.34 (m, 4H); 7.18-7.04 (m, 1H); 6.77 (s, 2H); 6.07 (d, J=5.1 Hz, 1H); 4.41-4.29 (m, 1H); 4.05-3.95 (m, 1H); 3.82-3.73 (m, 1H); 3.60-3.47 (m, 2H); 3.04-2.89 (m, 2H).

### Example 14: N-{3-[5-(2-Aminopyrimidin-4-yl)-2-tert-butyl-thiazol-4-yl]-5-chloro-2-fluorophenyl}-2,5-difluorobenzenesulfonamide

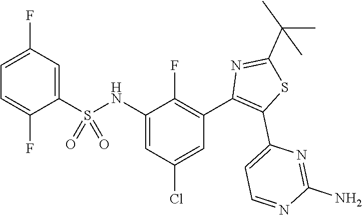 [SMILES: CC(C)(C)c1nc(-c2cc(Cl)cc(NS(=O)(=O)c3cc(F)ccc3F)c2F)c(-c2ccnc(N)n2)s1]

Step 1: N-{3-[2-tert-Butyl-5-(2-chloropyrimidin-4-yl)-thiazol-4-yl]-5-chloro-2-fluorophenyl}-2,5-difluorobenzenesulfonamide

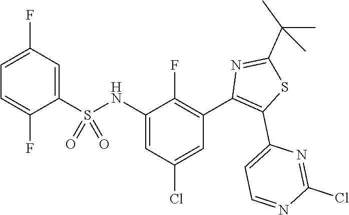 [SMILES: CC(C)(C)c1nc(-c2cc(Cl)cc(NS(=O)(=O)c3cc(F)ccc3F)c2F)c(-c2ccnc(Cl)n2)s1]

100 mg (0.21 mmol) of N-{5-chloro-3-[2-(2-chloropyrimidin-4-yl)-acetyl]-2-fluorophenyl}-2,5-difluorobenzenesulfonamide (described in example 13 step 3) are dissolved in 1 mL of anhydrous N,N′-dimethylacetamide at room temperature under argon, then 38 mg (0.21 mmol) of N-bromosuccinimide are added and the mixture is stirred for 30 min at room temperature. 25 mg (0.21 mmol) of 2,2-dimethylthiopropionamide are added under argon and the reaction mixture is heated at 80° C. for 30 min. The solution is cooled to room temperature, diluted with ethyl acetate. The solution is washed twice with water and once with brine, dried over anhydrous sodium sulfate, filtered and evaporated under reduced pressure. Crude product is purified by flash chromatography on a 10 g silica gel column using an ethyl acetate/hexane mixture as eluent. 70 mg of the title compound are obtained.

Yield: 61%.

MH⁺: 573.6; 575.6; 577.6.

Step 2: N-{3-[5-(2-Aminopyrimidin-4-yl)-2-tert-butyl-thiazol-4-yl]-5-chloro-2-fluorophenyl}-2,5-difluorobenzenesulfonamide

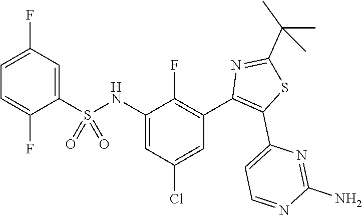 [SMILES: CC(C)(C)c1nc(-c2cc(Cl)cc(NS(=O)(=O)c3cc(F)ccc3F)c2F)c(-c2ccnc(N)n2)s1]

70 mg (0.12 mmol) of N-{3-[2-tert-Butyl-5-(2-chloropyrimidin-4-yl)-thiazol-4-yl]-5-chloro-2-fluorophenyl}-2,5-difluorobenzenesulfonamide (described in the previous step) are dissolved in 2 mL of a solution of ammonium hydroxide 28% and the solution is heated at 90° C. under microwave irradiation for 1 h. Mixture is diluted in 50 mL of a saturated solution of ammonium chloride and extracted 3 times with ethyl acetate. Combined organic layers are dried over anhydrous sodium sulfate, filtered and evaporated under reduced pressure. Crude product is purified by flash chromatography on a 10 g silica gel column using a dichloromethane/methanol mixture as eluent, followed by another flash chromatography on a 30 g C18 column with a water/acetonitrile mixture as eluent. 18 mg of the title compound are obtained.

Yield: 26%.

MH⁺: 554.7; 556.7.

¹H NMR (DMSO-d6, 400 MHz): δ 11.01 (br s, 1H); 8.04 (d, J=5.2 Hz, 1H); 7.63-7.37 (m, 5H); 6.76 (s, 2H); 5.99 (d, J=5.1 Hz, 1H); 1.40 (s, 9H).

### Example 15: N-{3-[5-(2-Aminopyrimidin-4-yl)-2-azetidin-2-yl-thiazol-4-yl]-2-fluorophenyl}-2,5-difluorobenzenesulfonamide

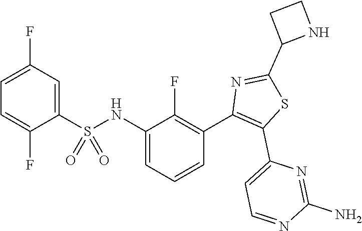 [SMILES: Nc1nccc(-c2sc(C3CCN3)nc2-c2cccc(NS(=O)(=O)c3cc(F)ccc3F)c2F)n1]

Step 1: 2-{5-(2-Chloropyrimidin-4-yl)-4-[3-(2,5-difluorobenzenesulfonylamino)-2-fluorophenyl]-thiazol-2-yl}-azetidine-1-carboxylic acid tert-butyl ester

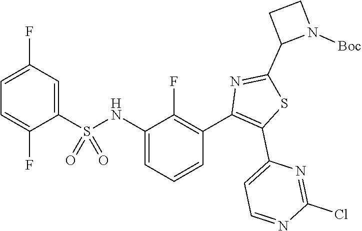 [SMILES: CC(C)(C)OC(=O)N1CCC1c1nc(-c2cccc(NS(=O)(=O)c3cc(F)ccc3F)c2F)c(-c2ccnc(Cl)n2)s1]

200 mg (0.46 mmol) of N-{3-[2-(2-chloropyrimidin-4-yl)-acetyl]-2-fluorophenyl}-2,5-difluorobenzenesulfonamide (described in example 1 step 3) are dissolved in 2 mL of anhydrous N,N′-dimethylacetamide at room temperature under argon, then 80 mg (0.46 mmol) of N-bromosuccinimide are added and the mixture is stirred for 30 min at room temperature. 98 mg (0.46 mmol) of 2-thiocarbamoylazetidine-1-carboxylic acid tert-butyl ester are added under argon and the reaction mixture is heated at 80° C. for 30 min. The solution is cooled to room temperature, diluted with ethyl acetate. The organic layer is washed twice with water and once with brine, dried over anhydrous sodium sulfate, filtered and evaporated under reduced pressure. Crude product is purified by flash chromatography on a 10 g silica gel column using an ethyl acetate/hexane mixture as eluent. 157 mg of the title compound are obtained.

Yield: 55%.

MH⁺: 638.8; 640.9.

Step 2: 2-{5-(2-Aminopyrimidin-4-yl)-4-[3-(2,5-difluorobenzenesulfonylamino)-2-fluorophenyl]-thiazol-2-yl}-azetidine-1-carboxylic acid tert-butyl ester

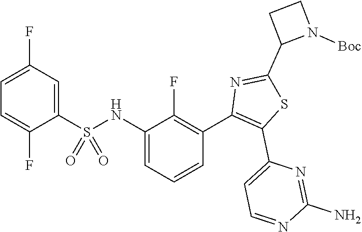 [SMILES: CC(C)(C)OC(=O)N1CCC1c1nc(-c2cccc(NS(=O)(=O)c3cc(F)ccc3F)c2F)c(-c2ccnc(N)n2)s1]

157 mg (0.24 mmol) of 2-{5-(2-chloropyrimidin-4-yl)-4-[3-(2,5-difluorobenzenesulfonylamino)-2-fluorophenyl]-thiazol-2-yl}-azetidine-1-carboxylic acid tert-butyl ester (described in the previous step) are dissolved in 2 mL of a solution of ammonium hydroxide 28% and the solution is heated at 90° C. under microwave irradiation for 1 h. Mixture is diluted in 50 mL of a saturated solution of ammonium chloride and extracted 3 times with ethyl acetate. Combined organic layers are dried over anhydrous sodium sulfate, filtered and evaporated under reduced pressure. Crude product is directly used in the next step without further purification (152 mg).

MH⁺: 619.9.

Step 3: N-{3-[5-(2-Aminopyrimidin-4-yl)-2-azetidin-2-yl-thiazol-4-yl]-2-fluorophenyl}-2,5-difluorobenzenesulfonamide

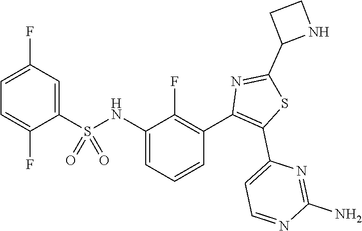 [SMILES: Nc1nccc(-c2sc(C3CCN3)nc2-c2cccc(NS(=O)(=O)c3cc(F)ccc3F)c2F)n1]

152 mg (0.24 mmol) of 2-{5-(2-aminopyrimidin-4-yl)-4-[3-(2,5-difluorobenzenesulfonylamino)-2-fluorophenyl]-thiazol-2-yl}-azetidine-1-carboxylic acid tert-butyl ester (described in the previous step) are dissolved in 1 mL of dichloromethane and 0.5 mL of trifluoroacetic acid are added. The solution is stirred for 1 h at room temperature. Then solvent is removed, ethyl acetate is added, and the solution is washed 3 times with a saturated solution of sodium bicarbonate. Organic layer is dried over anhydrous sodium sulfate, filtered and evaporated under reduced pressure. Crude product is purified by flash chromatography on a 10 g of silica gel column using a dichloromethane/methanol mixture as eluent, followed by another flash chromatography on a 30 g C18 column using a water/methanol mixture as eluent. 24 mg of the title compound are obtained as a slightly yellow solid.

Yield: 19% over 2 steps.

MH⁺: 519.7.

¹H NMR (DMSO-d6, 400 MHz): δ 8.00 (d, J=5.2 Hz, 1H); 7.52-7.30 (m, 4H); 7.10 (t, J=7.8 Hz, 1H); 7.06-6.97 (m, 1H); 6.72 (s, 2H); 5.98 (d, J=5.1 Hz, 1H); 5.20 (t, J=8.0 Hz, 1H); 3.76 (q, J=8.0 Hz, 1H); 3.41-3.31 (m, 1H); 2.71-2.61 (m, 1H); 2.46-2.35 (m, 1H).

### Example 16: N-{3-[5-(2-Aminopyrimidin-4-yl)-2-pyrrolidin-2-yl-thiazol-4-yl]-2-fluorophenyl}-2,5-difluorobenzenesulfonamide

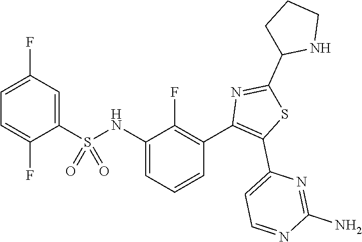 [SMILES: Nc1nccc(-c2sc(C3CCCN3)nc2-c2cccc(NS(=O)(=O)c3cc(F)ccc3F)c2F)n1]

Step 1: 2-{5-(2-Chloropyrimidin-4-yl)-4-[3-(2,5-difluorobenzenesulfonylamino)-2-fluorophenyl]-thiazol-2-yl}-pyrrolidine-1-carboxylic acid tert-butyl ester

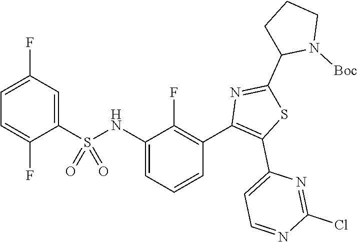 [SMILES: CC(C)(C)OC(=O)N1CCCC1c1nc(-c2cccc(NS(=O)(=O)c3cc(F)ccc3F)c2F)c(-c2ccnc(Cl)n2)s1]

191 mg (0.43 mmol) of N-{3-[2-(2-chloropyrimidin-4-yl)-acetyl]-2-fluoro-phenyl}-2,5-difluorobenzenesulfonamide (described in example 1 step 3) are dissolved in 2 mL of anhydrous N,N′-dimethylacetamide at room temperature under argon, then 77 mg (0.43 mmol) of N-bromosuccinimide are added and the mixture is stirred for 30 min at room temperature. 100 mg (0.43 mmol) of 2-thiocarbamoylpyrrolidine-1-carboxylic acid tert-butyl ester are added under argon and the reaction mixture is heated at 80° C. for 30 min. The solution is cooled to room temperature, diluted with ethyl acetate. The organic layer is washed twice with water and once with brine, dried over anhydrous sodium sulfate, filtered and evaporated under reduced pressure. Crude product is purified by flash chromatography on a 10 g silica gel column using an ethyl acetate/hexane mixture as eluent. 205 mg of the title compound are obtained.

Yield: 72%.

MH⁺: 652.9; 654.9.

Step 2: 2-{5-(2-Aminopyrimidin-4-yl)-4-[3-(2,5-difluorobenzenesulfonylamino)-2-fluorophenyl]-thiazol-2-yl}-pyrrolidine-1-carboxylic acid tert-butyl ester

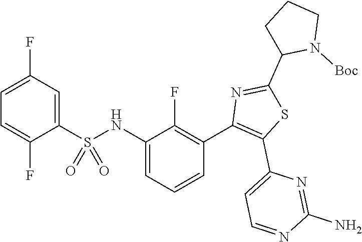 [SMILES: CC(C)(C)OC(=O)N1CCCC1c1nc(-c2cccc(NS(=O)(=O)c3cc(F)ccc3F)c2F)c(-c2ccnc(N)n2)s1]

205 mg (0.31 mmol) of 2-{5-(2-chloropyrimidin-4-yl)-4-[3-(2,5-difluorobenzenesulfonylamino)-2-fluorophenyl]-thiazol-2-yl}-pyrrolidine-1-carboxylic acid tert-butyl ester (described in the previous step) are dissolved in 2 mL of a solution of ammonium hydroxide 28% and the solution is heated at 90° C. under microwave irradiation for 1 h. Mixture is diluted in 50 mL of a saturated solution of ammonium chloride and extracted 3 times with ethyl acetate. Combined organic layers are dried over anhydrous sodium sulfate, filtered and evaporated under reduced pressure. Crude product is directly used in the next step without further purification (198 mg).

MH⁺: 633.9.

Step 3: N-{3-[5-(2-Aminopyrimidin-4-yl)-2-pyrrolidin-2-yl-thiazol-4-yl]-2-fluorophenyl}-2,5-difluorobenzenesulfonamide

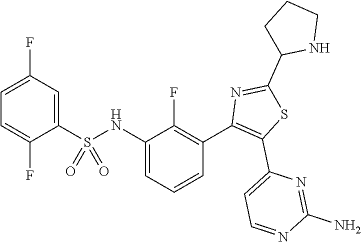 [SMILES: Nc1nccc(-c2sc(C3CCCN3)nc2-c2cccc(NS(=O)(=O)c3cc(F)ccc3F)c2F)n1]

198 mg (0.31 mmol) of 2-{5-(2-aminopyrimidin-4-yl)-4-[3-(2,5-difluorobenzenesulfonylamino)-2-fluorophenyl]-thiazol-2-yl}-pyrrolidine-1-carboxylic acid tert-butyl ester (described in the previous step) are dissolved in 1 mL of dichloromethane and 0.5 mL of trifluoroacetic acid is added. The solution is stirred for 1 h at room temperature. Then solvent is removed, ethyl acetate is added, and the solution is washed 3 times with a saturated solution of sodium bicarbonate. Organic layer is dried over anhydrous sodium sulfate, filtered and evaporated under reduced pressure. Crude product is purified by flash chromatography on a 10 g of silica gel column using a dichloromethane/methanol mixture as eluent, followed by another flash chromatography on a 30 g C18 column using a water/methanol mixture as eluent. 30 mg of the title compound are obtained as a slightly yellow solid.

Yield: 18% over 2 steps.

MH⁺: 533.7.

¹H NMR (DMSO-d6, 400 MHz): δ 7.98 (d, J=5.2 Hz, 1H); 7.52-7.31 (m, 4H); 7.19-7.05 (m, 2H); 6.71 (s, 2H); 5.93 (d, J=5.1 Hz, 1H); 4.62-4.55 (m, 1H); 3.10-2.95 (m, 2H); 2.29-2.17 (m, 1H); 1.96-1.84 (m, 1H); 1.84-1.74 (m, 2H).

### Example 17: N-{3-[5-(2-Aminopyrimidin-4-yl)-2-piperidin-2-yl-thiazol-4-yl]-2-fluorophenyl}-2,5-difluorobenzenesulfonamide

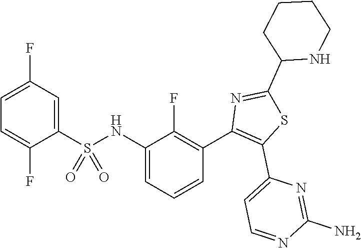 [SMILES: Nc1nccc(-c2sc(C3CCCCN3)nc2-c2cccc(NS(=O)(=O)c3cc(F)ccc3F)c2F)n1]

Step 1: 2-{5-(2-Chloropyrimidin-4-yl)-4-[3-(2,5-difluoro-benzenesulfonylamino)-2-fluorophenyl]-thiazol-2-yl}-piperidine-1-carboxylic acid tert-butyl ester

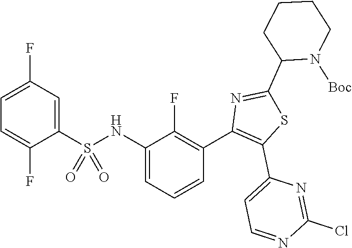 [SMILES: CC(C)(C)OC(=O)N1CCCCC1c1nc(-c2cccc(NS(=O)(=O)c3cc(F)ccc3F)c2F)c(-c2ccnc(Cl)n2)s1]

180 mg (0.41 mmol) of N-{3-[2-(2-chloropyrimidin-4-yl)-acetyl]-2-fluoro-phenyl}-2,5-difluorobenzenesulfonamide (described in example 1 step 3) are dissolved in 2 mL of anhydrous N,N′-dimethylacetamide at room temperature under argon, then 72 mg (0.41 mmol) of N-bromosuccinimide are added and the mixture is stirred for 30 min at room temperature. 100 mg (0.41 mmol) of 2-thiocarbamoylpiperidine-1-carboxylic acid tert-butyl ester are added under argon and the reaction mixture is heated at 80° C. for 30 min. The solution is cooled to room temperature, diluted with ethyl acetate. The solution is washed twice with water and once with brine, dried over anhydrous sodium sulfate, filtered and evaporated under reduced pressure. Crude product is purified by flash chromatography on a 10 g silica gel column using an ethyl acetate/hexane mixture as eluent. 169 mg of the title compound are obtained.

Yield: 62%.

MH⁺: 666.9; 668.9.

Step 2: 2-{5-(2-Aminopyrimidin-4-yl)-4-[3-(2,5-difluoro-benzenesulfonylamino)-2-fluorophenyl]-thiazol-2-yl}-piperidine-1-carboxylic acid tert-butyl ester

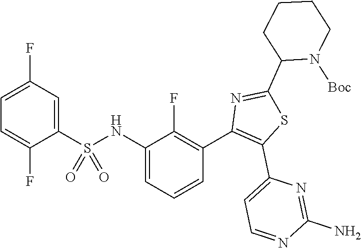 [SMILES: CC(C)(C)OC(=O)N1CCCCC1c1nc(-c2cccc(NS(=O)(=O)c3cc(F)ccc3F)c2F)c(-c2ccnc(N)n2)s1]

169 mg (0.25 mmol) of 2-{5-(2-chloropyrimidin-4-yl)-4-[3-(2,5-difluoro-benzenesulfonylamino)-2-fluorophenyl]-thiazol-2-yl}-piperidine-1-carboxylic acid tert-butyl ester (described in the previous step) are dissolved in 2 mL of a solution of ammonium hydroxide 28% and the solution is heated at 90° C. under microwave irradiation for 1 h. Mixture is diluted in 50 mL of a saturated solution of ammonium chloride and extracted 3 times with ethyl acetate. Combined organic layers are dried over anhydrous sodium sulfate, filtered and evaporated under reduced pressure. Crude product is directly used in the next step without further purification (163 mg).

MH⁺: 648.0.

Step 3: N-{3-[5-(2-Aminopyrimidin-4-yl)-2-piperidin-2-yl-thiazol-4-yl]-2-fluorophenyl}-2,5-difluorobenzenesulfonamide

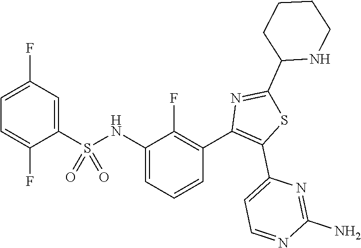 [SMILES: Nc1nccc(-c2sc(C3CCCCN3)nc2-c2cccc(NS(=O)(=O)c3cc(F)ccc3F)c2F)n1]

163 mg (0.25 mmol) of 2-{5-(2-aminopyrimidin-4-yl)-4-[3-(2,5-difluoro-benzenesulfonylamino)-2-fluorophenyl]-thiazol-2-yl}-piperidine-1-carboxylic acid tert-butyl ester (described in the previous step) are dissolved in 1 mL of dichloromethane and 0.5 mL of trifluoroacetic acid is added. The solution is stirred for 1 h at room temperature. Then solvent is removed, ethyl acetate is added, and the solution is washed 3 times with a saturated solution of sodium bicarbonate. Organic layer is dried over anhydrous sodium sulfate, filtered and evaporated under reduced pressure. Crude product is purified by flash chromatography on a 10 g of silica gel column using a dichloromethane/methanol mixture as eluent, followed by another flash chromatography on a 30 g C18 column using a water/methanol mixture as eluent. 62 mg of the title compound are obtained as a yellow solid.

Yield: 45% over 2 steps.

MH⁺: 547.8.

¹H NMR (DMSO-d6, 400 MHz): δ 8.02 (d, J=5.2 Hz, 1H); 7.51-7.31 (m, 4H); 7.09 (t, J=7.8 Hz, 1H); 7.03-6.93 (m, 1H); 6.75 (s, 2H); 6.00 (d, J=5.1 Hz, 1H); 4.35-4.24 (m, 1H); 3.19-3.09 (m, 1H); 2.88-2.77 (m, 1H); 2.16-2.06 (m, 1H); 1.86-1.76 (m, 1H); 1.70-1.43 (m, 4H).

### Example 18: N-{3-[5-(2-Aminopyrimidin-4-yl)-2-piperazin-2-yl-thiazol-4-yl]-2-fluorophenyl}-2,5-difluorobenzenesulfonamide

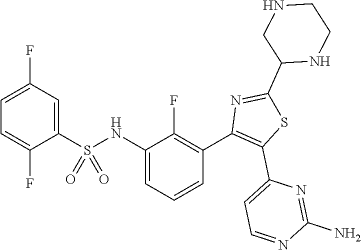 [SMILES: Nc1nccc(-c2sc(C3CNCCN3)nc2-c2cccc(NS(=O)(=O)c3cc(F)ccc3F)c2F)n1]

Step 1: 2-Carbamoylpiperazine-1,4-dicarboxylic acid di-tert-butyl ester

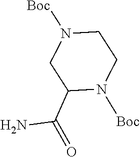 [SMILES: CC(C)(C)OC(=O)N1CCN(C(=O)OC(C)(C)C)C(C(N)=O)C1]

229 mg (1.00 mmol) of 2-carbamoylpiperazine-1-carboxylic acid tert-butyl ester are dissolved in 3 mL of anhydrous tetrahydrofuran under argon, then 229 mg (1.05 mmol) of di-tert-butyl dicarbonate are added and the mixture is stirred for 1 h at room temperature. The solution is then diluted with ethyl acetate. The organic layer is washed twice with a saturated solution of sodium bicarbonate and once with brine. Organic layer is dried over anhydrous sodium sulfate, filtered and evaporated under reduced pressure. Crude product is purified by flash chromatography on a 10 g silica gel column using a dichloromethane/methanol mixture as eluent. 329 mg of the title compound are obtained as a white solid foam.

Yield: Quantitative.

MH⁺: 330.6.

Step 2: 2-Thiocarbamoylpiperazine-1,4-dicarboxylic acid di-tert-butyl ester

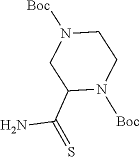 [SMILES: CC(C)(C)OC(=O)N1CCN(C(=O)OC(C)(C)C)C(C(N)=S)C1]

360 mg (1.09 mmol) of 2-carbamoylpiperazine-1,4-dicarboxylic acid di-tert-butyl ester (described in the previous step) are dissolved in 4 mL of tetrahydrofuran, then 380 mg (0.94 mmol) of Lawesson's reagent are added and the mixture is stirred at 60° C. for 4 h. Reaction mixture is cooled to room temperature and the solvent is removed under reduced pressure. The residue is quenched with a saturated solution of sodium bicarbonate, and extracted with ethyl acetate. Combined organic are dried over anhydrous sodium sulfate, filtered and evaporated under reduced pressure. Crude product is purified by flash chromatography on a 10 g of silica gel column using a dichloromethane/methanol mixture as eluent. 261 mg of the title compound are obtained as a white solid.

Yield: 70%.

MH⁺: 346.7.

Step 3: 2-{5-(2-Chloropyrimidin-4-yl)-4-[3-(2,5-difluorobenzenesulfonylamino)-2-fluorophenyl]-thiazol-2-yl}-piperazine-1,4-dicarboxylic acid di-tert-butyl ester

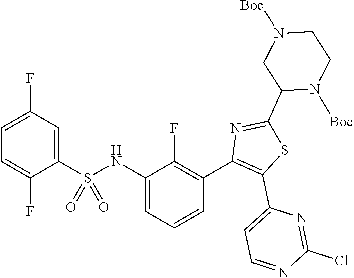 [SMILES: CC(C)(C)OC(=O)N1CCN(C(=O)OC(C)(C)C)C(c2nc(-c3cccc(NS(=O)(=O)c4cc(F)ccc4F)c3F)c(-c3ccnc(Cl)n3)s2)C1]

191 mg (0.43 mmol) of N-{3-[2-(2-chloropyrimidin-4-yl)-acetyl]-2-fluoro-phenyl}-2,5-difluorobenzenesulfonamide (described in example 1 step 3) are dissolved in 2 mL of anhydrous N,N′-dimethylacetamide at room temperature under argon, then 78 mg (0.43 mmol) of N-bromosuccinimide are added and the mixture is stirred for 30 min at room temperature. 150 mg (0.43 mmol) of 2-thiocarbamoylpiperazine-1,4-dicarboxylic acid di-tert-butyl ester (described in the previous step) are added under argon and the reaction mixture is heated at 80° C. for 30 min. The solution is cooled to room temperature, diluted with ethyl acetate. The solution is washed twice with water and once with brine, dried over anhydrous sodium sulfate, filtered and evaporated under reduced pressure. Crude product is purified by flash chromatography on a 10 g silica gel column using an ethyl acetate/hexane mixture as eluent. 132 mg of the title compound are obtained as a white solid.

Yield: 40%.

MH⁺: 768.0; 770.0.

Step 4: 2-{5-(2-Aminopyrimidin-4-yl)-4-[3-(2,5-difluorobenzenesulfonylamino)-2-fluorophenyl]-thiazol-2-yl}-piperazine-1,4-dicarboxylic acid di-tert-butyl ester

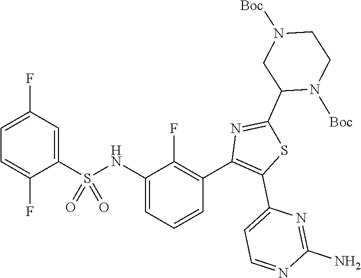 [SMILES: CC(C)(C)OC(=O)N1CCN(C(=O)OC(C)(C)C)C(c2nc(-c3cccc(NS(=O)(=O)c4cc(F)ccc4F)c3F)c(-c3ccnc(N)n3)s2)C1]

132 mg (0.17 mmol) of 2-{5-(2-aminopyrimidin-4-yl)-4-[3-(2,5-difluorobenzenesulfonylamino)-2-fluorophenyl]-thiazol-2-yl}-piperazine-1,4-dicarboxylic acid di-tert-butyl ester (described in the previous step) are dissolved in 2 mL of a solution of ammonium hydroxide 28% and the solution is heated at 90° C. under microwave irradiation for 1 h. Mixture is diluted in 50 mL of a saturated solution of ammonium chloride and extracted 3 times with ethyl acetate. Combined organic layers are dried over anhydrous sodium sulfate, filtered and evaporated under reduced pressure. Crude product is directly used in the next step without further purification (122 mg).

MH⁺: 749.2.

Step 5: N-{3-[5-(2-Aminopyrimidin-4-yl)-2-piperazin-2-yl-thiazol-4-yl]-2-fluorophenyl}-2,5-difluorobenzenesulfonamide

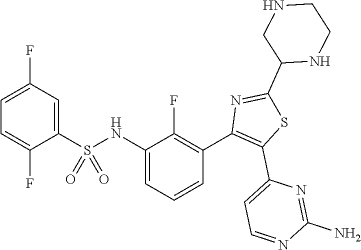 [SMILES: Nc1nccc(-c2sc(C3CNCCN3)nc2-c2cccc(NS(=O)(=O)c3cc(F)ccc3F)c2F)n1]

122 mg (0.16 mmol) of 2-{5-(2-chloropyrimidin-4-yl)-4-[3-(2,5-difluorobenzenesulfonylamino)-2-fluorophenyl]-thiazol-2-yl}-piperazine-1,4-dicarboxylic acid di-tert-butyl ester (described in the previous step) are dissolved in 1 mL of dichloromethane and 0.5 mL of trifluoroacetic acid is added. The solution is stirred for 1 h at room temperature. Then solvent is removed, ethyl acetate is added, and the solution is washed 3 times with a saturated solution of sodium bicarbonate. Organic layer is dried over anhydrous sodium sulfate, filtered and evaporated under reduced pressure. Crude product is purified by flash chromatography on a 10 g of silica gel column using a dichloromethane/methanol mixture as eluent, followed by another flash chromatography on a 30 g C18 column with a water/methanol mixture as eluent. 14 mg of the title compound are obtained as a slightly yellow solid.

Yield: 16% over 2 steps.

MH⁺: 548.6.

¹H NMR (DMSO-d6, 400 MHz): δ 8.00 (d, J=5.2 Hz, 1H); 7.51-7.42 (m, 1H); 7.34-7.20 (m, 3H); 6.92 (t, J=7.8 Hz, 1H); 6.71 (s, 2H); 6.69-6.62 (m, 1H); 6.05 (d, J=5.1 Hz, 1H); 4.27-4.18 (m, 1H); 3.47-3.36 (m, 1H); 3.12-3.00 (m, 2H); 2.99-2.77 (m, 3H).

### Example 19: N-{3-[5-(2-Aminopyrimidin-4-yl)-2-(6,6-dimethylmorpholin-3-yl)thiazol-4-yl]-2-fluorophenyl}-2,5-difluorobenzenesulfonamide

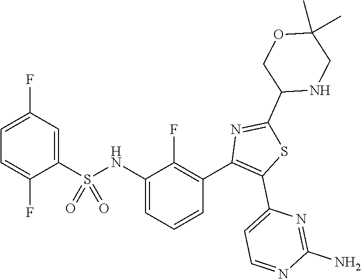 [SMILES: CC1(C)CNC(c2nc(-c3cccc(NS(=O)(=O)c4cc(F)ccc4F)c3F)c(-c3ccnc(N)n3)s2)CO1]

Step 1: 5-Carbamoyl-2,2-dimethylmorpholine-4-carboxylic acid tert-butyl ester

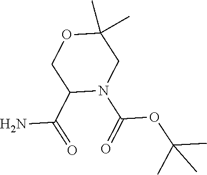 [SMILES: CC(C)(C)OC(=O)N1CC(C)(C)OCC1C(N)=O]

150 mg (0.580 mmol) of 6,6-dimethylmorpholine-3,4-dicarboxylic acid 4-tert-butyl ester are dissolved in 3 mL of anhydrous tetrahydrofuran under argon, then 200 μL (1.16 mmol) of N,N′-diisopropylethylamine and 220 mg (0.580 mmol) of HATU are added and the mixture is stirred for 15 min at room temperature. 2.9 mL (1.16 mmol) of ammonia 0.4 M in dioxane are added and the mixture is stirred for 3 h at room temperature. The solution is then diluted with ethyl acetate and washed with a saturated solution of sodium bicarbonate, followed by a saturated solution of NH₄Cl and finally by brine. Organic layer is dried over anhydrous sodium sulfate, filtered and evaporated under reduced pressure. Crude product is purified by flash chromatography on a 10 g silica gel column using a dichloromethane/methanol mixture as eluent. 182 mg of the title compound are obtained as colorless oil.

Yield: Quantitative.

MH⁺: 259.5.

Step 2: 2,2-Dimethyl-5-thiocarbamoylmorpholine-4-carboxylic acid tert-butyl ester

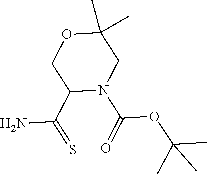 [SMILES: CC(C)(C)OC(=O)N1CC(C)(C)OCC1C(N)=S]

172 mg (0.666 mmol) of 5-carbamoyl-2,2-dimethylmorpholine-4-carboxylic acid tert-butyl ester (described in the previous step) are dissolved in 3 mL of anhydrous tetrahydrofuran under argon, then 232 mg (0.573 mmol) of Lawesson's reagent are added and the mixture is stirred at 60° C. for 2 h 30. Reaction mixture is cooled to room temperature and the solvent is removed under reduced pressure. The residue is quenched with a saturated solution of sodium bicarbonate, and extracted with ethyl acetate. Combined organic layers are dried over anhydrous sodium sulfate, filtered and evaporated under reduced pressure. Crude product is purified by 2 successive flash chromatographies on 10 g of silica gel columns: a/ dichloromethane/methanol mixture as eluent, b/ ethyl acetate/hexane mixture as eluent. 70 mg of the title compound are obtained as a colorless gel.

Yield: 38%.

MH⁺: 275.5.

Step 3: 5-{5-(2-Chloropyrimidin-4-yl)-4-[3-(2,5-difluorobenzenesulfonyl-amino)-2-fluorophenyl]-thiazol-2-yl}-2,2-dimethylmorpholine-4-carboxylic acid tert-butyl ester

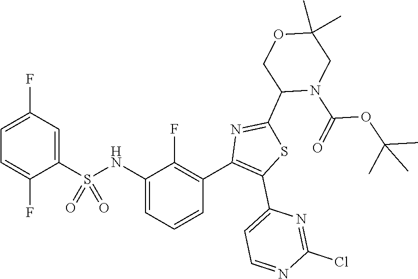 [SMILES: CC(C)(C)OC(=O)N1CC(C)(C)OCC1c1nc(-c2cccc(NS(=O)(=O)c3cc(F)ccc3F)c2F)c(-c2ccnc(Cl)n2)s1]

110 mg (0.249 mmol) of N-{3-[2-(2-chloropyrimidin-4-yl)-acetyl]-2-fluoro-phenyl}-2,5-difluorobenzenesulfonamide (described in Example 1, step 3) are dissolved in 2 mL of anhydrous N,N′-dimethylacetamide at room temperature under argon, then 44 mg (0.247 mmol) of N-bromosuccinimide are added and the mixture is stirred for 30 min at room temperature. 68 mg (0.248 mmol) of 2,2-dimethyl-5-thiocarbamoyl-morpholine-4-carboxylic acid tert-butyl ester (described in the previous step) are added under argon and the reaction mixture is heated at 80° C. for 30 min. The solution is cooled to room temperature, diluted with ethyl acetate. The solution is washed 5 times with a mixture of water/brine (1/1), dried over anhydrous sodium sulfate, filtered and evaporated under reduced pressure. Crude product is purified by flash chromatography on a 10 g silica gel column using an ethyl acetate/hexane mixture as eluent. 98 mg of the title compound are obtained as an off-white solid.

Yield: 26%.

MH⁺: 696.6; 698.6.

Step 4: 5-{5-(2-Aminopyrimidin-4-yl)-4-[3-(2,5-difluorobenzenesulfonyl-amino)-2-fluorophenyl]-thiazol-2-yl}-2,2-dimethylmorpholine-4-carboxylic acid tert-butyl ester

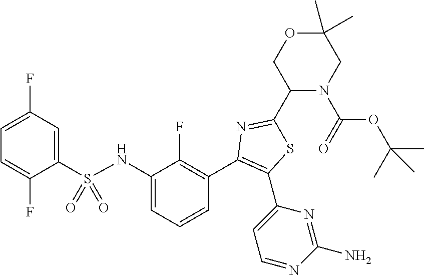 [SMILES: CC(C)(C)OC(=O)N1CC(C)(C)OCC1c1nc(-c2cccc(NS(=O)(=O)c3cc(F)ccc3F)c2F)c(-c2ccnc(N)n2)s1]

96 mg (0.138 mmol) of 5-{5-(2-chloropyrimidin-4-yl)-4-[3-(2,5-difluoro-benzenesulfonylamino)-2-fluoro-phenyl]-thiazol-2-yl}-2,2-dimethylmorpholine-4-carboxylic acid tert-butyl ester (described in the previous step) are dissolved in 1.5 mL of a solution of ammonium hydroxide 28% and then stirred at 90° C. under microwave irradiation for 1 h. Mixture is diluted in a mixture of water/brine (1/1) and extracted 3 times with ethyl acetate. Combined organic layers are dried over anhydrous sodium sulfate, filtered and evaporated under reduced pressure. Crude product is directly used in the next step without further purification (58 mg of the title compound, orange solid).

MH⁺: 677.7.

Step 5: N-{3-[5-(2-Aminopyrimidin-4-yl)-2-(6,6-dimethylmorpholin-3-yl)-thiazol-4-yl]-2-fluorophenyl}-2,5-difluorobenzenesulfonamide

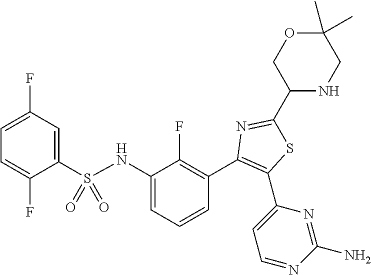 [SMILES: CC1(C)CNC(c2nc(-c3cccc(NS(=O)(=O)c4cc(F)ccc4F)c3F)c(-c3ccnc(N)n3)s2)CO1]

58 mg (0.160 mmol) of N-{3-[5-(2-aminopyrimidin-4-yl)-2-(6,6-dimethyl-morpholin-3-yl)-thiazol-4-yl]-2-fluorophenyl}-2,5-difluorobenzenesulfonamide (described in the previous step) are dissolved in 1 mL of dichloromethane and 0.5 mL of trifluoroacetic acid is added. The solution is stirred for 1 h at room temperature. Then solvent is removed and the residue is mixed with a saturated solution of sodium bicarbonate. This aqueous mixture is 3 times extracted using ethyl acetate. Combined organic layers are dried over anhydrous sodium sulfate, filtered and evaporated under reduced pressure. Crude product is purified by flash chromatography on a 10 g of silica gel column using a dichloromethane/methanol mixture as eluent, followed by another flash chromatography on a 30 g C18 column with a water/methanol mixture as eluent. Finally, a third column (10 g of silica, ethyl acetate/hexane) is performed. 10 mg of the title compound are obtained as a yellow solid.

Yield: 13% over 2 steps.

MH⁺: 577.6.

¹H NMR (DMSO-d6, 400 MHz): δ 10.74 (br s, 1H); 7.99 (d, J=5.1 Hz, 1H); 7.67-7.00 (m, 6H); 6.74 (s, 2H); 5.88 (s, 1H); 4.06-3.98 (m, 1H); 3.80-3.73 (m, 1H); 3.66-3.57 (m, 1H); 2.74 (d, J=12.2 Hz, 1H); 2.69 (d, 1H); 1.25 (s, 3H); 1.14 (s, 3H).

### Example 20: N-{3-[5-(2-Aminopyrimidin-4-yl)-2-(4-cyclopropylpiperazin-2-yl)-thiazol-4-yl]-2-fluorophenyl}-2,5-difluorobenzenesulfonamide

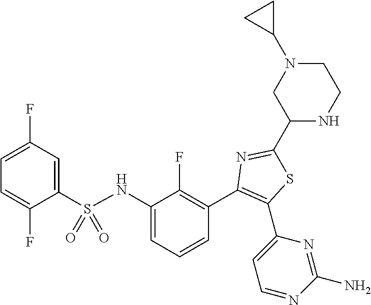 [SMILES: Nc1nccc(-c2sc(C3CN(C4CC4)CCN3)nc2-c2cccc(NS(=O)(=O)c3cc(F)ccc3F)c2F)n1]

Step 1: 4-Cyclopropylpiperazine-1,2-dicarboxylic acid 1-tert-butyl ester 2-methyl ester

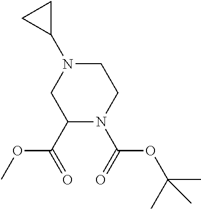 [SMILES: COC(=O)C1CN(C2CC2)CCN1C(=O)OC(C)(C)C]

500 mg (2.05 mmol) of piperazine-1,2-dicarboxylic acid 1-tert-butyl ester 2-methyl ester are dissolved in 35 mL of anhydrous methanol under argon. 617 μL (3.07 mmol) of (1-ethoxycyclopropoxy)-trimethylsilane, 351 μL (6.14 mmol) of acetic acid and 206 mg (3.27 mmol) of sodium cyanoborohydride are added and the mixture is stirred at 60° C. for 16 h. The solvent is removed under reduced pressure and the residue is directly purified by flash chromatography on a 10 g of silica gel column using a dichloromethane/methanol mixture as eluent. Remaining traces of acetic acid are removed by dissolving the purified compound in a saturated solution of sodium bicarbonate and extracting it 3 times with ethyl acetate. Combined organic layer are dried over anhydrous sodium sulfate, filtered and evaporated under reduced pressure. 569 mg of the title compound are obtained as a colorless gel.

Yield: 98%.

MH⁺: 285.6.

Step 2: 4-Cyclopropylpiperazine-1,2-dicarboxylic acid 1-tert-butyl ester

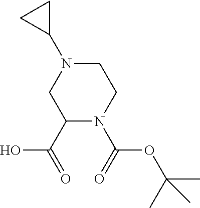 [SMILES: CC(C)(C)OC(=O)N1CCN(C2CC2)CC1C(=O)O]

569 mg (2.00 mmol) of 4-cyclopropylpiperazine-1,2-dicarboxylic acid 1-tert-butyl ester 2-methyl ester (described in the previous step) are dissolved in 12 mL of methanol/water (1/1) mixture. 105 mg (4.4 mmol) of lithium hydroxide are added and the mixture is stirred at room temperature for 16 h. The mixture is concentrated under reduced pressure, diluted with water and 3 times extracted with dichloromethane. The aqueous layer is acidified to pH 2-3 using HCl 2 N, then saturated with sodium chloride and 18 times extracted by ethyl acetate. Crude product is directly used in the next step without further purification (492 mg of the title compound, colorless gel).

Yield: 91%.

MH⁺: 271.5.

Step 3: 2-Carbamoyl-4-cyclopropylpiperazine-1-carboxylic acid tert-butyl ester

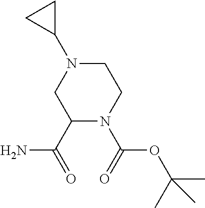 [SMILES: CC(C)(C)OC(=O)N1CCN(C2CC2)CC1C(N)=O]

492 mg (1.82 mmol) of 4-cyclopropylpiperazine-1,2-dicarboxylic acid 1-tert-butyl ester (described in the previous step) are dissolved in 9 mL of anhydrous tetrahydrofuran under argon, then 629 μL (3.64 mmol) of N,N′-diisopropylethylamine and 692 mg (1.82 mmol) of HATU are added and the mixture is stirred for 20 min at room temperature. 9.1 mL (3.64 mmol) of ammonia 0.4 M solution in dioxane are added and the mixture is stirred for 16 h at room temperature. The solution is then diluted with a saturated solution of sodium bicarbonate and 3 times extracted using ethyl acetate. Combined organic layers are dried over anhydrous sodium sulfate, filtered and evaporated under reduced pressure. Crude product is purified by flash chromatography on a 30 g silica gel column using a dichloromethane/methanol mixture as eluent. 532 mg of the title compound are obtained as a colorless gel.

Yield: Quantitative.

MH⁺: 270.5.

Step 4: 4-Cyclopropyl-2-thiocarbamoylpiperazine-1-carboxylic acid tert-butyl ester

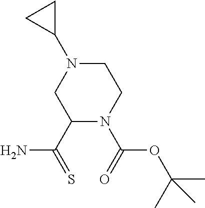 [SMILES: CC(C)(C)OC(=O)N1CCN(C2CC2)CC1C(N)=S]

532 mg (1.98 mmol) of 2-carbamoyl-4-cyclopropylpiperazine-1-carboxylic acid tert-butyl ester (described in the previous step) are dissolved in 7 mL of anhydrous tetrahydrofuran under argon, then 687 mg (1.70 mmol) of Lawesson's reagent are added and the mixture is stirred at 60° C. for 16 h. Reaction mixture is cooled to room temperature and quenched with a saturated solution of sodium bicarbonate, and 3 times extracted by ethyl acetate. Combined organic layers are dried over anhydrous sodium sulfate, filtered and evaporated under reduced pressure. Crude product is purified by flash chromatography over 30 g of silica gel column using a dichloromethane/methanol mixture as eluent. 127 mg of the title compound are obtained as yellow oil.

Yield: 24%.

MH⁺: 286.5.

Step 5: 2-{5-(2-Chloropyrimidin-4-yl)-4-[3-(2,5-difluorobenzenesulfonylamino)-2-fluorophenyl]-thiazol-2-yl}-4-cyclopropylpiperazine-1-carboxylic acid tert-butyl ester

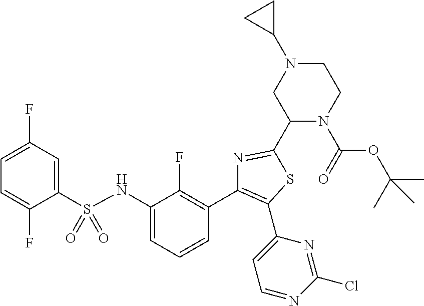 [SMILES: CC(C)(C)OC(=O)N1CCN(C2CC2)CC1c1nc(-c2cccc(NS(=O)(=O)c3cc(F)ccc3F)c2F)c(-c2ccnc(Cl)n2)s1]

97 mg (0.445 mmol) of N-{3-[2-(2-chloropyrimidin-4-yl)-acetyl]-2-fluoro-phenyl}-2,5-difluorobenzenesulfonamide (described in Example 1, step 3) are dissolved in 1 mL of anhydrous N,N′-dimethylacetamide at room temperature under argon, then 79 mg (0.445 mmol) of N-bromosuccinimide are added and the mixture is stirred for 30 min at room temperature. 127 mg (0.445 mmol) of 4-cyclopropyl-2-thiocarbamoylpiperazine-1-carboxylic acid tert-butyl ester (described in the previous step) are added under argon and the reaction mixture is heated at 80° C. for 30 min. The solution is cooled to room temperature, diluted with ethyl acetate. The solution is washed 5 times with a mixture of water/brine (1/1), dried over anhydrous sodium sulfate, filtered and evaporated under reduced pressure. Crude product is purified by flash chromatography on a 10 g silica gel column using an ethyl acetate/hexane mixture as eluent. 158 mg of the title compound are obtained as a yellow gel.

Yield: 50%.

MH⁺: 707.7; 709.7.

Step 6: 2-{5-(2-Aminopyrimidin-4-yl)-4-[3-(2,5-difluorobenzenesulfonyl-amino)-2-fluorophenyl]-thiazol-2-yl}-4-cyclopropylpiperazine-1-carboxylic acid tert-butyl ester

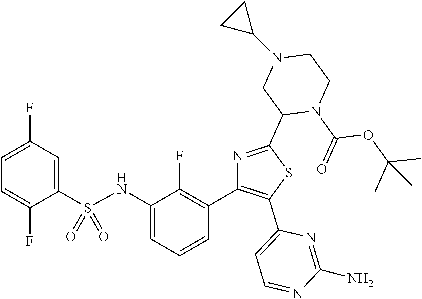 [SMILES: CC(C)(C)OC(=O)N1CCN(C2CC2)CC1c1nc(-c2cccc(NS(=O)(=O)c3cc(F)ccc3F)c2F)c(-c2ccnc(N)n2)s1]

58 mg (0.223 mmol) of 2-{5-(2-chloropyrimidin-4-yl)-4-[3-(2,5-difluoro-benzenesulfonylamino)-2-fluorophenyl]-thiazol-2-yl}-4-cyclopropylpiperazine-1-carboxylic acid tert-butyl ester (described in the previous step) are dissolved in 5 mL of a solution of ammonium hydroxide 28% and the solution is heated at 90° C. under microwave irradiation for 1 h 20. Mixture is diluted in a mixture of water/brine (1/1) and extracted 3 times with ethyl acetate. Combined organic layers are dried over anhydrous sodium sulfate, filtered and evaporated under reduced pressure. Crude product is directly used in the next step without further purification (110 mg of the title compound, yellow gel).

MH⁺: 688.7.

Step 7: N-{3-[5-(2-Aminopyrimidin-4-yl)-2-(4-cyclopropylpiperazin-2-yl)-thiazol-4-yl]-2-fluorophenyl}-2,5-difluorobenzenesulfonamide

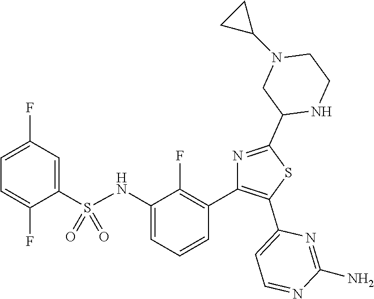 [SMILES: Nc1nccc(-c2sc(C3CN(C4CC4)CCN3)nc2-c2cccc(NS(=O)(=O)c3cc(F)ccc3F)c2F)n1]

110 mg (0.160 mmol) of 2-{5-(2-aminopyrimidin-4-yl)-4-[3-(2,5-difluoro-benzenesulfonylamino)-2-fluorophenyl]-thiazol-2-yl}-4-cyclopropylpiperazine-1-carboxylic acid tert-butyl ester (described in the previous step) are dissolved in 3 mL of dichloromethane and 1.5 mL of trifluoroacetic acid is added. The solution is stirred for 1 h at room temperature. Then solvent is removed and the residue is quenched with a saturated solution of sodium bicarbonate. This aqueous mixture is 3 times extracted by ethyl acetate. Combined organic layers are dried over anhydrous sodium sulfate, filtered and evaporated under reduced pressure. Crude product is purified by flash chromatography on a 10 g of silica gel column using a dichloromethane/methanol mixture as eluent, followed by another flash chromatography on a 30 g C18 column with a water/methanol mixture as eluent. 56 mg of the title compound are obtained as a yellow solid.

Yield: 43% over 2 steps.

MH⁺: 588.7.

¹H NMR (DMSO-d6, 400 MHz): δ 7.99 (d, J=5.2 Hz, 1H); 7.55-7.35 (m, 4H); 7.27-7.17 (m, 2H); 6.73 (s, 2H); 5.90 (d, J=5.1 Hz, 1H); 4.10-4.03 (m, 1H); 3.11-3.04 (m, 1H); 2.97-2.89 (m, 1H); 2.82-2.70 (m, 2H); 2.44-2.30 (m, 2H); 1.72-1.63 (m, 1H); 0.47-0.27 (m, 4H).

### Example 21: BRAF Binding Assay

To assess compounds capacity to bind BRAF, LanthaScreen Biochemical Kinase Binding assay from Life technologies was used according to manufacturer's instructions. Briefly, white 384-well plates containing 160 nL of 100× compound solution in 100% DMSO, 3.84 μL kinase buffer, 8.0 μL 2× BRAF/Eu-anti-GST mixture and 4.0 μL 4× Tracer were used. Plates were shaken for 30 seconds and incubated for 60 minutes at room temperature. A plate reader was used to read fluorescence. In this assay, BRAF enzyme was used at a concentration of 5 nM and Eu-anti-GST antibody at a concentration of 2 nM. Tracer 178 was used at a concentration of 20 nM (Kd of 20 nM). Kinase buffer consisted of 50 mM HEPES pH 7.5, 0.01% BRIJ-35, 10 mM MgCl₂, 1 mM EGTA. Compounds IC₅₀ were determined with a 3-fold serial dilution (10 point titrations in duplicate).

BRAF binding of selected compounds (“BRAF IC₅₀”) is reported in table 2 as followed:

All tested compounds exhibit a capacity to bind BRAF kinase.

In particular, compounds having an activity designated as “A” provided an IC₅₀ value <10 nM. Compounds having an activity designated as “B” provided an IC₅₀ value between 10 nM and 25 nM. Compounds having an activity designated as “C” provided an IC₅₀ value between 25 nM and 50 nM. Compounds having an activity designated as “D” provided an IC₅₀ value between 50 nM and 100 nM. Compounds having an activity designated as “E” provided an IC₅₀ value >100 nM.

TABLE 2
BRAF binding potencies (IC₅₀) of selected compounds

| Compound | BRAF IC₅₀ |
|---|---|
| 13 | A |
| 14 | A |

### Example 22: BRAF V599E Binding Assay

To assess compounds capacity to bind BRAF V599E, LanthaScreen Biochemical Kinase Binding assay from Life technologies was used according to manufacturer's instructions. Briefly, white 384-well plates containing 160 nL of 100× compound solution in 100% DMSO, 3.84 μL kinase buffer, 8.0 μL 2× BRAF V599E/Eu-anti-GST mixture and 4.0 μL 4× Tracer were used. Plates were shaken for 30 seconds and incubated for 60 minutes at room temperature. A plate reader was used to read fluorescence. In this assay, BRAF V599E enzyme was used at a concentration of 5 nM and Eu-anti-GST antibody at a concentration of 2 nM. Tracer 178 was used at a concentration of 20 nM (Kd of 33 nM). Kinase buffer consisted of 50 mM HEPES pH 7.5, 0.01% BRIJ-35, 10 mM MgCl2, 1 mM EGTA. Compounds IC₅₀ were determined with a 3-fold serial dilution (10 point titrations in duplicate).

BRAF V599E binding of selected compounds (“BRAF V599E IC₅₀”) is reported in table 3 as followed:

All tested compounds exhibit a capacity to bind BRAF V599E kinase.

In particular, compounds having an activity designated as “A” provided an IC₅₀ value <10 nM. Compounds having an activity designated as “B” provided an IC₅₀ value between 10 nM and 25 nM. Compounds having an activity designated as “C” provided an IC₅₀ value between 25 nM and 50 nM. Compounds having an activity designated as “D” provided an IC₅₀ value between 50 nM and 100 nM. Compounds having an activity designated as “E” provided an IC₅₀ value >100 nM.

TABLE 3
BRAF V599E binding potencies (IC₅₀) of selected compounds

| Compound | BRAF V599E IC₅₀ |
|---|---|
| 1 | A |
| 2 | A |
| 3 | A |
| 4 | A |
| 8 | A |
| 13 | A |
| 14 | A |
| 15 | A |
| 16 | A |
| 17 | A |
| 18 | A |

### Example 23: Cell Line Proliferation Assay

A375 cell proliferation was assessed using the standard MTT assay as previously described (Delfosse et al, 2012).

Briefly, A375 cells were seeded at a density of 500 cells per well in 96-well tissue culture plates and grown in test culture medium. Test compounds were added 24 h after seeding. Cell lines were incubated for 4 days at 37° C. After the incubation period, the medium containing test compounds was removed and replaced by test culture medium containing 0.4 mg/mL MTT. After incubation (4 h), viable cells cleaved the MTT tetrazolium ring into a dark blue formazan reaction product, whereas dead cells remained colorless. The MTT-containing medium was gently removed and DMSO was added to each well. After shaking, the plates were read in absorbance at 540 nm. Tests were performed in quadriplicate in at least 3 independent experiments. Data were expressed as % of the maximal activity obtained in absence of ligand.

Cell proliferation of selected compounds (“BRAF V599E IC50”) is reported in table 4 relative to the reference compound Dabrafenib.

Most of the tested compounds exhibit a capacity to inhibit A375 cell proliferation although to a nearly identical or slightly lower level compared to the reference compound.

TABLE 4
A375 cell proliferation inhibition are reported as a
ratio of potencies (IC50) of selected compounds against
the potency of the reference compound dabrafenib

| | IC₅₀ dabrafenib/IC₅₀ compound |
|---|---|
| Compound | BRAF activity |
| Dabrafenib | 1 |
| Encorafenib | 1.16 |
| 1 | 0.53 |
| 2 | 0.79 |
| 5 | 0.9 |
| 7 | 0.25 |
| 13 | 0.69 |
| 14 | 0.76 |
| 17 | 0.5 |

### Example 24: PXR Transactivation Assay

PXR activity was characterized using an already established HG5LN GAL4-hPXR reporter cell lines (Lemaire et al, 2007). In brief, HG5LN cells were obtained by integration of a GAL4-responsive gene (GAL4RE5-bGlob-Luc-SV-Neo) in HeLa cells (Seimandi et al., 2005). The HG5LN GAL4(DBD)-hPXR(LBD) cell line was obtained by transfecting HG5LN cells with a plasmid [pSG5-GAL4(DBD)-hPXR(LBD)-puro], which enables the expression of the DNA binding domain of the yeast activator GAL4 (Met1-Ser147) fused to the ligand binding domain of hPXR (Met107-Ser434) and confers resistance to puromycin.

HG5LN and HG5LN GAL4-hPXR cells were cultured in Dulbecco's Modified EagleMedium: Nutrient Mixture F-12 (DMEM/F-12) containing phenol red and 1 g/L glucose and supplemented with 5% fetal bovine serum, 100 units/mL of penicillin, 100 μg/mL of streptomycin and 1 mg/mL geneticinin a 5% CO₂ humidified atmosphere at 37° C. HG5LN GAL4-hPXR cells were cultured in the same medium supplemented with 0.5 μg/mL puromycin.

For transactivation experiments, HG5LN and HG5LN-PXR were seeded at a density of 25,000 cells per well in 96-well white opaque tissue culture plates (Greiner CellStar) in Dulbecco's Modified Eagle Medium: Nutrient Mixture F-12 (DMEM/F-12) without phenol red and 1 g/L glucose and supplemented with 5% stripped fetal bovine serum, 100 units/mL of penicillin, 100 μg/mL of streptomycin (test medium). Compounds to be tested were added 24 h later, and cells were incubated at 37° C. for 16 h. At the end of the incubation period, culture medium was replaced with test medium containing 0.3 mM luciferin. Luciferase activity was measured for 2 s in intact living cells using a MicroBeta Wallac luminometer (PerkinElmer). Tests were performed in quadruplicate in at least 3 independent experiments. Data were expressed as % of the maximal activity obtained in absence of ligand (HG5LN cells) or with SR12813 3 μM (HG5LN PXR cells).

PXR transactivation of selected compounds is reported in table 5 relative to the reference compound Dabrafenib.

All tested compounds, but the chlorinated analog of Dabrafenib (GL214), exhibit a much lower activation of PXR in the reporter assay compared to the reference compound dabrafenib.

TABLE 5
PXR activation reporter assay

| | EC₅₀ dabrafenib/EC₅₀ compound |
|---|---|
| Compound | PXR activity |
| Dabrafenib | 1 |
| Encorafenib | 0.012 |
| 1 | 0.0018 |
| 2 | 0.067 |
| 5 | 0.00042 |
| 7 | 0.0012 |
| 13 | 0.0024 |
| 14 | 0.43 |
| 15 | 0.0024 |
| 16 | 0.00045 |
| 17 | 0.01 |
| 18 | 0.0012 |

1. A compound of Formula I: 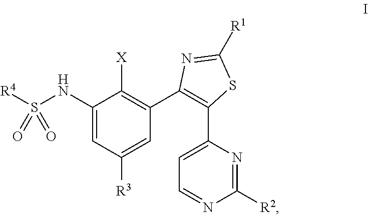
or a pharmaceutically acceptable salt or a solvate thereof,
wherein
X is halogen;
R¹ is selected from the group consisting of C1-C6-alkyl, amino-C1-C6-alkyl, piperidinyl, morpholinyl, piperazinyl, pyrrolidinyl and azetidinyl, said amino-C1-C6-alkyl, piperidinyl, morpholinyl, piperazinyl, pyrrolidinyl and azetidinyl being linked to the thiazol ring through a carbon atom and optionally substituted by C1-C6-alkyl, C3-C6-cycloalkyl or C1-C4-alkyloxycarbonyl;
R² is selected from the group consisting of C1-C6-alkyl, halogen and NHR⁵, wherein R⁵ is selected from the group consisting of H, —C(O)—C1-C6-alkyl, —C(O)—C1-C6-alkenyl and —C(O)—C1-C6-alkynyl;
R³ is selected from the group consisting of H, C1-C6-alkyl and halogen; and
R⁴ is selected from the group consisting of C1-C6-alkyl and dihalogenoaryl;
with the proviso that R¹ is not C1-C6-alkyl when one of R², R³ and R⁴ is C1-C6-alkyl or when R³ is H.

2. The compound according to claim 1, wherein X is fluoro.

3. The compound according to claim 1, wherein R² is NHR⁵, wherein R⁵ is selected from the group consisting of H, —C(O)Me, —C(O)—CH═CH₂ and —C(O)—C≡CH.

4. The compound according to claim 1, wherein R³ is H or chloro.

5. The compound according to claim 1, wherein R⁴ is selected from the group consisting of C1-C6-alkyl and 2,5-dihalogenophenyl.

6. The compound according to claim 1, having the Formula II: 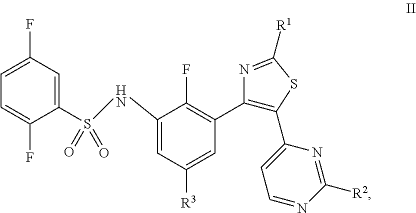
or a pharmaceutically acceptable salt or a solvate thereof,
wherein
R¹, R² and R³ are as defined in claim 1.

7. The compound according to claim 1, having the Formula III: 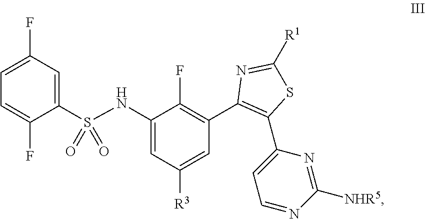
or a pharmaceutically acceptable salt or a solvate thereof,
wherein
R¹, R³ and R⁵ are as defined in claim 1.

8. The compound according to claim 1, having the Formula IV: 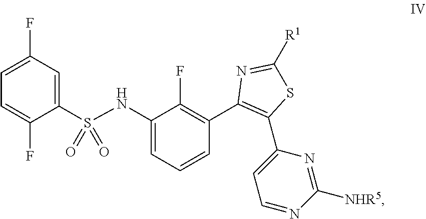
or a pharmaceutically acceptable salt or a solvate thereof,
wherein
R¹ and R⁵ are as defined in claim 1.

9. The compound according to claim 1, having the Formula V: 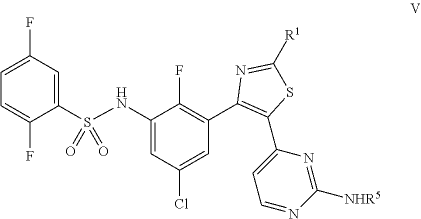
or a pharmaceutically acceptable salt or a solvate thereof,
wherein
R¹ and R⁵ are as defined in claim 1.

10. The compound according to claim 1, selected from the group consisting of:
N-{3-[5-(2-aminopyrimidin-4-yl)-2-morpholin-3-yl-thiazol-4-yl]-2-fluorophenyl}-2,5-difluorobenzenesulfonamide;
N-{3-[5-(2-aminopyrimidin-4-yl)-2-(1-cyclopropylpiperidin-4-yl)-thiazol-4-yl]-2-fluorophenyl}-2,5-difluorobenzenesulfonamide;
N-{3-[5-(2-aminopyrimidin-4-yl)-2-piperidin-3-yl-thiazol-4-yl]-2-fluorophenyl}-2,5-difluorobenzenesulfonamide;
N-{3-[2-(3-aminopropyl)-5-(2-aminopyrimidin-4-yl)-thiazol-4-yl]-2-fluorophenyl}-2,5-difluorobenzenesulfonamide trifluoroacetate;
N-(4-{2-(1-cyclopropylpiperidin-4-yl)-4-[3-(2,5-difluorobenzenesulfonylamino)-2-fluorophenyl]-thiazol-5-yl}-pyrimidin-2-yl)-acetamide;
N-(4-{4-[3-(2,5-difluorobenzenesulfonylamino)-2-fluorophenyl]-2-piperidin-3-yl-thiazol-5-yl}-pyrimidin-2-yl)-acetamide trifluoroacetate;
N-(4-{4-[3-(2,5-difluorobenzenesulfonylamino)-2-fluorophenyl]-2-morpholin-3-yl-thiazol-5-yl}-pyrimidin-2-yl)-acetamide;
N-(4-{2-(3-aminopropyl)-4-[3-(2,5-difluorobenzenesulfonylamino)-2-fluorophenyl]-thiazol-5-yl}-pyrimidin-2-yl)-acetamide;
N-(4-{4-[3-(2,5-difluorobenzenesulfonylamino)-2-fluorophenyl]-2-morpholin-3-yl-thiazol-5-yl}-pyrimidin-2-yl)-acrylamide trifluoroacetate;
N-(4-{2-(3-aminopropyl)-4-[3-(2,5-difluorobenzenesulfonylamino)-2-fluorophenyl]-thiazol-5-yl}-pyrimidin-2-yl)-acrylamide trifluoroacetate;
3-{5-(2-acryloylaminopyrimidin-4-yl)-4-[3-(2,5-difluorobenzenesulfonylamino)-2-fluorophenyl]-thiazol-2-yl}-piperidine-1-carboxylic acid tert-butyl ester;
butane-2-sulfonic acid {3-[5-(2-aminopyrimidin-4-yl)-2-piperidin-4-yl-thiazol-4-yl]-2-fluorophenyl}-amide;
N-{3-[5-(2-aminopyrimidin-4-yl)-2-morpholin-3-yl-thiazol-4-yl]-5-chloro-2-fluorophenyl}-2,5-difluorobenzenesulfonamide;
N-{3-[5-(2-aminopyrimidin-4-yl)-2-tert-butyl-thiazol-4-yl]-5-chloro-2-fluorophenyl}-2,5-difluorobenzenesulfonamide;
N-{3-[5-(2-aminopyrimidin-4-yl)-2-azetidin-2-yl-thiazol-4-yl]-2-fluorophenyl}-2,5-difluorobenzenesulfonamide;
N-{3-[5-(2-aminopyrimidin-4-yl)-2-pyrrolidin-2-yl-thiazol-4-yl]-2-fluorophenyl}-2,5-difluorobenzenesulfonamide;
N-{3-[5-(2-aminopyrimidin-4-yl)-2-piperidin-2-yl-thiazol-4-yl]-2-fluorophenyl}-2,5-difluorobenzenesulfonamide;
N-{3-[5-(2-aminopyrimidin-4-yl)-2-piperazin-2-yl-thiazol-4-yl]-2-fluorophenyl}-2,5-difluorobenzenesulfonamide;
N-{3-[5-(2-aminopyrimidin-4-yl)-2-(6,6-dimethylmorpholin-3-yl)thiazol-4-yl]-2-fluorophenyl}-2,5-difluorobenzenesulfonamide; and
N-{3-[5-(2-aminopyrimidin-4-yl)-2-(4-cyclopropylpiperazin-2-yl)thiazol-4-yl]-2-fluorophenyl}-2,5-difluorobenzenesulfonamide.

11. A pharmaceutical composition comprising a compound according to claim 1 or a pharmaceutically acceptable salt or solvate thereof and at least one pharmaceutically acceptable excipient.

12. A method of therapeutic treatment in humans or animals, comprising the step of administering to a patient in need thereof a therapeutically effective amount of a compound according to claim 1, or a pharmaceutically acceptable salt or solvate thereof.

13. A method of treating or preventing a disease or disorder associated with deregulated protein kinase activity comprising the step of administering to a patient in need thereof a therapeutically effective amount of a compound according to claim 1, or a pharmaceutically acceptable salt or solvate thereof.

14. The method according to claim 13, wherein the protein kinase is B-RAF or a mutant form thereof.

15. A method of treating or preventing cancer, comprising the step of administering to a patient in need thereof a therapeutically effective amount of a compound according to claim 1, or a pharmaceutically acceptable salt or solvate thereof.

16. The method according to claim 15, wherein the cancer is selected from the group consisting of melanoma, lung cancer, colorectal cancer, gastro-intestinal stromal cancer and pancreatic cancer.
